(19) United States
(12) Patent Application Publication
(10) Pub. No.: US 2026/0224296 A1
(43) Pub. Date: Aug. 6, 2026

(54) SYSTEMS AND METHODS FOR MEDICAL INTERVENTION

(71) Applicant: Fall River Laboratories, Inc., Mountain View, CA (US)

(72) Inventors: Eric C. Browy, Meridian, ID (US); David C. Lundmark, Los Altos, CA (US)

(73) Assignee: Fall River Laboratories, Inc., Mountain View, CA (US)

(21) Appl. No.: 19/420,682

(22) Filed: Dec. 15, 2025

Related U.S. Application Data
(60) Provisional application No. 63/733,873, filed on Dec. 13, 2024.

(51) Int. Cl. A61B 34/20; A61B 34/30

(52) U.S. Cl. CPC A61B 34/20; A61B 34/30; A61B 2034/2051; A61B 2034/2055; A61B 2034/2063; A61B 2034/301

(57) ABSTRACT

One embodiment is directed to an elongate instrument navigation system for conducting a medical procedure on a patient, comprising: an elongate instrument comprising an elongate body coupled to two or more discretely positioned magnetic steering nodes, the elongate instrument comprising an insertion length selected such that a distal portion may be inserted to reach a position of a targeted interventional structure within the body of the patient, and a cross-sectional size profile configured to be navigated through one or more tissue structures of the patient as the patient is positioned in an operating environment which may be characterized by a global coordinate system; a computing system operatively coupled to the elongate instrument and configured to present a control interface to an operator; and two or more magnetic field sources, each of which is controllably movably coupled relative to the patient by a robotic coupling assembly operatively coupled to the computing system; wherein in response to commands provided by the operator to the computing system, the computing system is configured to precisely navigate a portion of the elongate body of the elongate instrument relative to the one or more tissue structures of the patient by causing each of the two or more magnetic field sources to be positioned and oriented relative to the patient and relative to the two or more magnetic steering nodes of the elongate instrument using each of the robotic coupling assemblies.

### RELATED APPLICATION

This application claims priority to and incorporates by reference in its entirety U.S. patent application Ser. No. 63/733,873 filed Dec. 13, 2024.

### FIELD OF THE INVENTION

This invention relates generally to the field of medical diagnostics and intervention, and more specifically to diagnostic and interventional medical instrumentation configured to be guided, at least in part, via precision-vectored magnetic flux, as well as methods and configurations related thereto.

### BACKGROUND

The need for advancement in medical interventional and diagnostic technology remains critical in countries throughout the world, and factors such as aging populations, rising rates of chronic disease, global health infrastructure development, minimally invasive techniques, government initiatives, and patient preferences have enhanced the associated demand. Referring to FIG. 1A, typically, certain diagnostic and/or interventional steps are associated with an operating room (2) or surgery suite, wherein patient (6) and associated operators (4) may convene with enhanced environmental controls and appropriate instrumentation (8), such as a scalpel or electrocautery tool. Referring to FIG. 1B, conventional open surgery often involves one or more direct intervention tools (8, 9) which may be held by the surgeon or operator team (4, 5) to directly address the patient (6) through a wound or access portal created through the skin of the patient. Referring to FIG. 2A, certain minimally invasive techniques have been developed to conduct surgical interventions through small transcutaneous portals into the patient, as shown in FIG. 2A wherein an operator (4) is using both hands to manipulate tools (10, 12; such as a camera or “laparoscope” and remote grasping tool) within a gas-filled portion of the interior of the patient, such as the abdomen (14). Referring to FIG. 2B, somewhat similar suites of tools (10, 12, 16, 18) have been utilized, such as through discrete and different transcutaneous access portals (here intercostal, or between-rib, access portals) to gain access to aspects of the thorax for surgical intervention pertaining to certain tissue structures of the thorax, such as aspects of the lungs, for example.

Referring to FIG. 3A, common portal areas or regions (22) may be utilized in certain procedures to focus transcutaneous access through a particular area or region as shown. In the configuration of FIG. 3A, an operator (4) is conducting on a patient (6) what may be termed “single port surgery”, such that a plurality of tools or instruments (10, 12, 16) are guided and utilized for an intervention through a single surgically-created portal (20). Referring to FIG. 3B, in another variation, a common portal area or region (22) may be utilized to co-locate a plurality of distinct but proximally-located surgically-created portals (24, 26, 28) to provide access for a plurality of distinct tools or instruments (16, 12, 10, respectively).

Referring to FIG. 4, certain flexible instruments (36) may be utilized for diagnostic and/or interventional procedures within tissue structures such as the lungs (30) of a patient (6) by accessing using lumens or structures that already exist within the patient, such as the nose or nasal (38) passages, the trachea (32), and the interconnected bronchi (34).

Referring to FIG. 5, certain flexible instruments (36) may be utilized for diagnostic and/or interventional procedures pertaining to the upper gastrointestinal tract of a patient (6), such as by accessing the stomach (46) of the patient through lumens or structures that already exist within the patient, such as the nose or nasal (38) passages, the mouth (40), and the esophagus (42).

Referring to FIGS. 6A and 6B, certain flexible instruments, such as those referred to as “colonoscope” (52) varieties, may be utilized to access aspects of the lower gastrointestinal system of the patient (6), such as the small intestine (48), such as by insertion through lumens or structures that already exist within the patient, such as the large intestine (50; also known as the colon). FIG. 6B illustrates a colonoscope (52) that has been navigated partially through the large intestine (50) of a patient to conduct a procedure wherein a working tool (54; may comprise, for example, a biopsy needle and/or grasper tool) that is controllably movable relative to a distal portion of the colonoscope (52; may comprise, for example, an image capture device 56 configured to provide image information pertaining to the associated environment and/or tools 54). Conventionally one of the challenges with such configurations has been precision controllability of the instruments relative to the targeted tissue.

Referring to FIGS. 7A and 7B, robotic surgery systems (58) have been developed to provide enhanced capabilities and levels of control through electromechanical guidance and association with image information. The configuration (58) illustrated in FIG. 7A comprises a plurality of discretely controllable instruments (62). Referring to FIG. 7B, a so-called “single port” (60) variation of a system such as that illustrated in FIG. 7A may be configured to deliver a plurality (64) of discretely-controllable instruments through a single conduit or instrument delivery assembly (66) such that a single surgical portal into the patient may be utilized.

Referring to FIG. 8, relatively small flexible instruments which may be known, for example, as “catheters” (74), may be navigated into the vessels of the cardiovascular system of a patient (6) to conduct diagnostic and/or interventional procedures. For example, FIG. 8 illustrates a catheter (74) which has been passed through a surgically-created transcutaneous access portal to the femoral artery (68) of the patient (6). In conducting such a procedure, an access portal region (70) may be targeted for the transcutaneous “cut-down” access based upon optimal proximity to a particular portion of targeted anatomy such as a particular portion of the femoral artery (68), and a proximal instrument assembly (76) coupled to the catheter may be inserted and/or positioned at or adjacent to the access region (70) to provide controlled valving, infusion, working tool access, and the like, such as via use of one or more valve assemblies called “Luer” assemblies. With such procedures, dilation of instruments (i.e. starting with relatively small diameter instrumentation such as a needle or guidewire and successively advancing up to a larger instrumentation diameter such as that of a catheter or Luer assembly) may be utilized to establish desired operational access geometry.

Referring to FIG. 9, a specialized operating room called a “catheter lab” or so-called “cath lab” (78) is illustrated. Such a configuration may be utilized, for example, with a procedure such as that illustrated in FIG. 8. A patient may be situated on an operating table (100) so that operators may have ready access to the patient, and so that imaging instrumentation such as a fluoroscopic imaging system comprising a so-called “C-arm” (94; may be coupled to the ceiling 90 of the cath lab 78, such as via a coupling assembly 92) intercoupling a radiographic imaging transmitter (96) and receiver (98) may be positioned for utilization during the procedure. An assembly of display devices (86) may be positioned within the cath lab (78) to provide views of imaging data from the fluoroscopy system or other systems, such as an ultrasound system (shown in FIG. 10; element 80 is a mobile ultrasound system which is operatively coupled to an ultrasound transducer 82, and typically also one more display devices 84), other forms of radiography, magnetic resonance imaging data, computed tomography data, or other systems, to provide an operating team with visualization of pertinent structures and/or instrumentation.

Referring to FIG. 11A, a steerable catheter instrument (102) may be configured such that loading and/or relative motion imparted by an operator at a handle portion (104) may be associated with controlled motion at the distal portion (106) of the catheter (102), such as via the use of intercoupled pull or push wires or members coupled through the instrument to a portion such as the distal portion (106) of the catheter (102).

Referring to FIG. 11B, a motorized catheter driver assembly (110), such as that designed by entities such as Hansen Medical, Inc. using the tradename Sensei™ and/or Magellan™, may be utilized to impart loads and/or relative motion to an intercoupled catheter instrument (102) to provide electromechanical steerability of portions of the catheter instrument.

Referring to FIGS. 12A-12D, as an alternative to steering configurations such as those described above in relative to FIGS. 11A and 11B which may involve tensioning of pullwires and/or pushing or pushwires or similar mechanical or electromechanical control elements, an applied magnetic field (120), also conventionally denotated with a “B”, may be utilized to impart loading upon a ferromagnetic element (114; i.e., an iron-containing element), such as one positioned at an instrument (112) distal end (116) as shown, to provide for a steering load or torque (118) selected to controllably bend or steer the instrument (112). FIGS. 12B and 12C illustrate systems, such as those developed by the entity Stereotaxis, Inc., configured to utilize a relatively large pair of magnetic field elements (122, 124) to provide some controllability to a flexible instrument (112) such as that shown in FIG. 12C or 12D within an effective magnetic field control volume, such as a spherical volume of about 8 inches.

There is a continued need for sophisticated interventional and diagnostic systems which are capable of enhanced control and capability for use in minimally-invasive diagnostic and interventional procedures of various types in various tissue structures and systems.

### BRIEF DESCRIPTION OF THE DRAWINGS

FIGS. 1A-1B illustrate aspects of conventional surgical procedures.

FIGS. 2A-2B and 3A-3B illustrate aspects of minimally invasive surgical procedures utilizing instruments such as laparoscopes.

FIG. 4 illustrates various aspects of an elongate instrument configuration utilized to address various aspects of the pulmonary system.

FIG. 5 illustrates various aspects of an elongate instrument configuration utilized to address various aspects of the upper gastrointestinal system;

FIGS. 6A and 6B illustrate various aspects of an elongate instrument configuration utilized to address various aspects of the lower gastrointestinal system.

FIGS. 7A and 7B illustrate aspects of a conventional robotic surgery system.

FIG. 8 illustrates aspects of a catheterization of a cardiovascular system of a human to provide endovascular access for instrumentation.

FIGS. 9 and 10 illustrate aspects of instrumentation which may be utilized in a catheterization laboratory, such as fluoroscopy and ultrasonic imaging systems.

FIGS. 11A-11B illustrate aspects of a robotic catheter system operated by components such as pullwires.

FIGS. 12A-12D illustrate various aspects of a conventional magnetically-assisted interventional system.

FIGS. 13A-13I illustrate various aspects of multi-node, multi-field magnetic steerability configurations which may be utilized to controllably navigate and shape elongate portions of an elongate instrument, such as when interfaced with aspects of a cardiovascular system of a patient.

FIGS. 14 and 15A-15D illustrate various aspects of precision magnetic steerability configurations.

FIG. 16 illustrates a plurality of robotic manipulators or arms which may be utilized to position and orient a plurality of magnetic field sources.

FIG. 17 illustrates various aspects of electromechanical engagement which may be utilized to control aspects of an instrument relative to a patient.

FIGS. 18A-18B illustrate various aspects of magnetic field guidance configurations which may be integrated with imaging modalities such as ultrasound.

FIGS. 19A-19E and 20A-20B illustrate various aspects of system configurations and components which may be integrated into a multi-node, multi-field instrumentation configuration.

FIGS. 21A-21C illustrate various aspects of instrument motion control configurations which may be utilized to provide controlled motion degrees of freedom relative to a patient or coordinate system, such as insertion, retraction, and/or roll.

FIGS. 22A-22H and 23A-23B illustrate various aspects of various cross-sectional instrument configurations.

FIGS. 24 and 25 illustrate various aspects of encoder and reader configurations which may be utilized to determine roll and/or insertion/retraction positioning of an associated elongate instrument.

FIGS. 26A-26F, 27, 28A-28D, and 29A-29E illustrate various aspects of elongate instrumentation configurations for engaging tissue and/or other structures.

FIGS. 30A-30D illustrate various aspects of configurations featuring robotically positioned and/or oriented magnetic field sources which may be utilized to controllably guide an elongate instrument, and which may be integrated with one or more imaging system elements such as one or more ultrasound transducers.

FIG. 31 illustrates a configuration wherein a content of magnetically-responsive material within a portion of an elongate instrument may be controllably varied.

FIGS. 32A-32D, 33A-33C, 34, 35, 36, and 37A-37B illustrate various aspects of system configurations wherein controlled mechanical stimulation, such as by controlled oscillatory motion, may be utilized to assist in specific operation of an elongate instrument.

FIGS. 38A-38B, and 39A-39H illustrate various aspects of cardiovascular procedures which may involve distal protection via utilization of an expandable and retractable protective configuration.

FIGS. 40, 41A-41B, 42A-42B, and 43A-43C illustrate various aspects of system configurations which may be utilized for an interventional procedure, such as diagnostic aspects which may be utilized preoperatively.

FIGS. 44, 45, and 46 illustrate various aspects of arterial anatomy and associated catherization/entry (such as at the femoral artery) for elongate instrumentation to conduct a procedure.

FIGS. 47A-47B illustrate various paths which may be utilized to address various aspects of targeted anatomy of a patient using the arterial vasculature.

FIGS. 48, 49A-49B, 50A-50B, 51A-51F illustrate various aspects of controlled approaches to reach targeted locations within the anatomy via multi-node controlled navigation of elongate instrumentation.

FIGS. 52A-52P illustrate various aspects of controlled approaches and instrumentation configurations to reach targeted locations within the anatomy via multi-node controlled navigation of elongate instrumentation, such as configurations featuring controlled stability or stabilization features.

FIGS. 53A-53M illustrate various aspects of a thrombus or clot removal procedure utilizing multi-node controlled navigation of elongate instrumentation, as well as multi-modal operation, such as via one or more controlled modes of thrombus or clot distruption (such as via oscillatory motion), motion configuration, stability enhancement, and/or risk mitigation.

FIG. 54 illustrates various aspects of a mobile configuration of a multi-node controlled system for precision navigation and operation of elongate instrumentation.

FIGS. 55A-55C and 56A-56C illustrate various aspects of operational configurations which may incorporate aspects of multi-node controlled navigation of elongate instrumentation.

FIGS. 57-66 illustrate various aspects of operational and/or training configurations which may incorporate aspects of multi-node controlled navigation of elongate instrumentation.

FIGS. 67, 68A-68C, 69A-69B, and 70-72 illustrate various aspects of multi-node controlled instrumentation configuration wherein one or more node may be controllably repositioned.

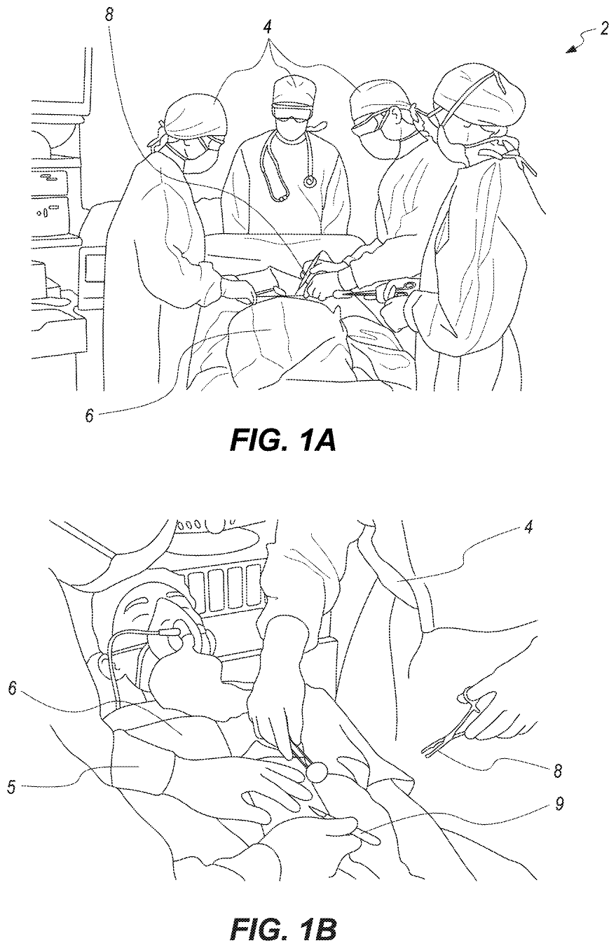

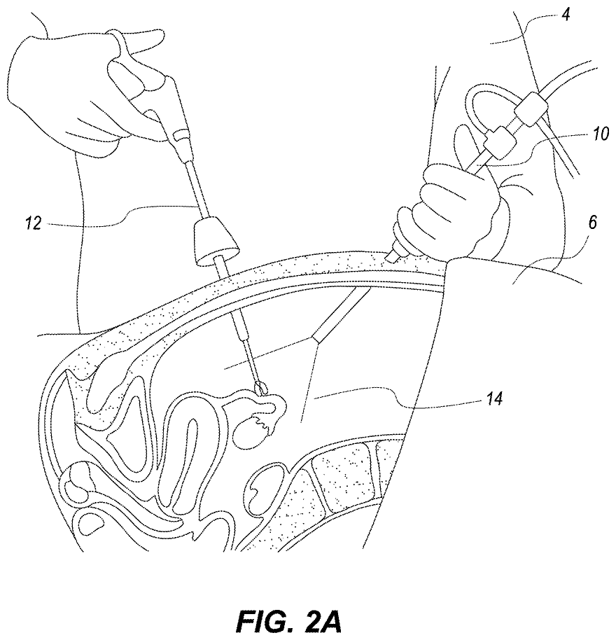

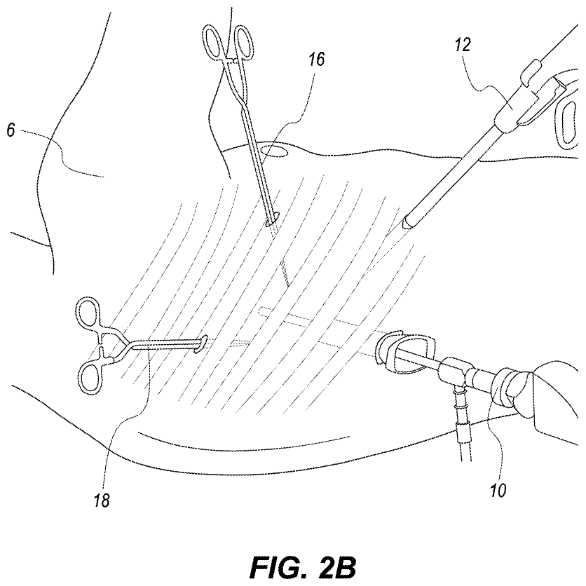

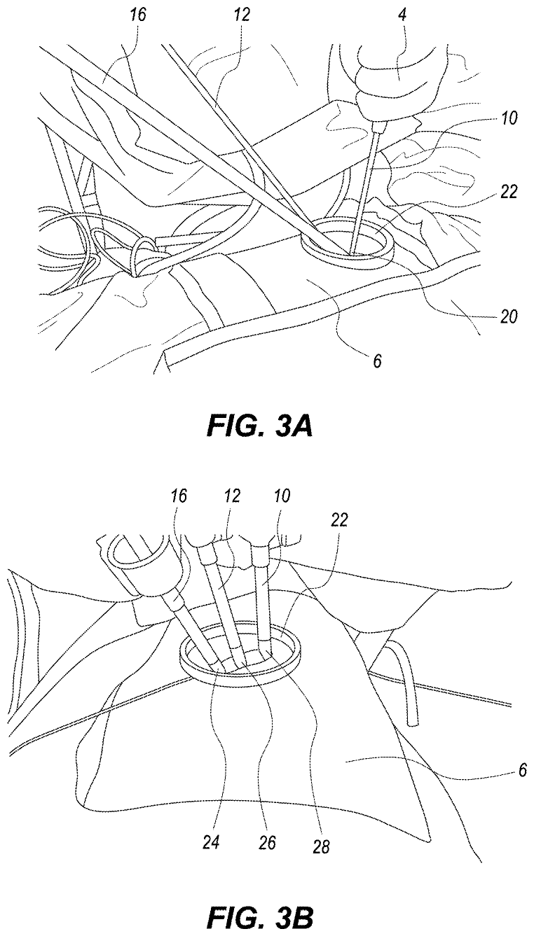

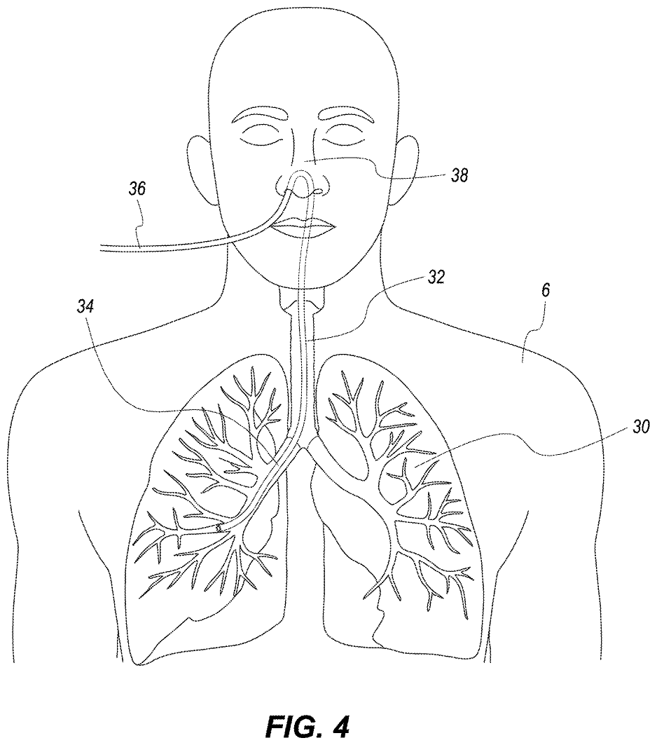

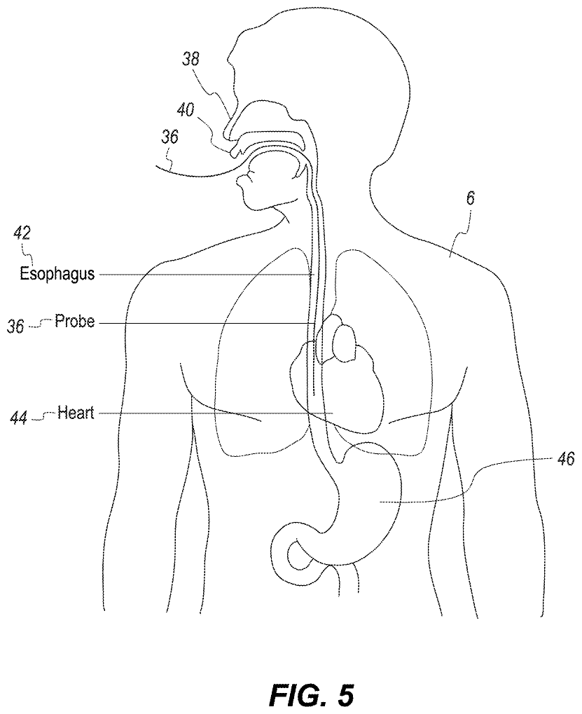

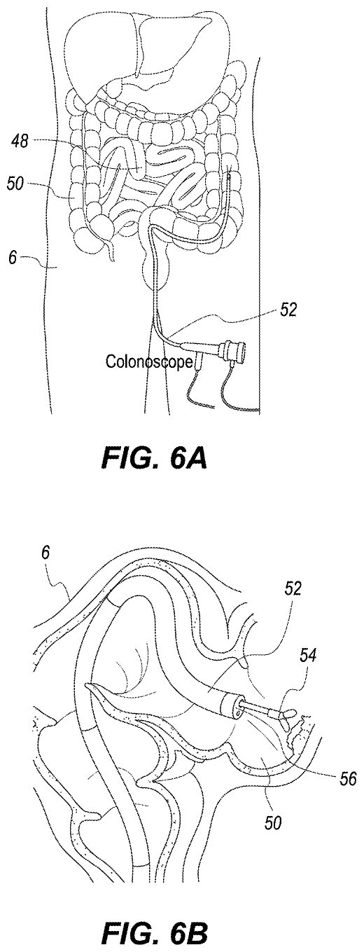

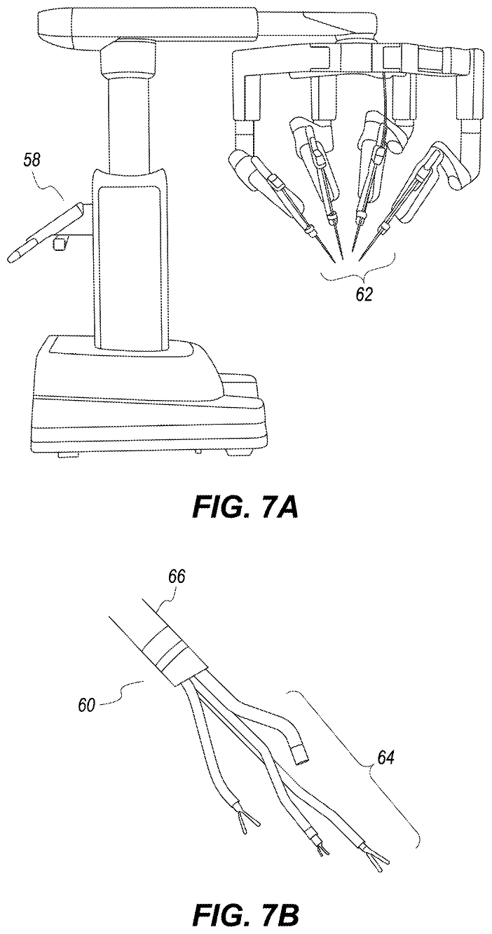

### SUMMARY

One embodiment is directed to an elongate instrument navigation system for conducting a medical procedure on a patient, comprising: an elongate instrument comprising an elongate body coupled to two or more discretely positioned magnetic steering nodes, the elongate instrument comprising an insertion length selected such that a distal portion may be inserted to reach a position of a targeted interventional structure within the body of the patient, and a cross-sectional size profile configured to be navigated through one or more tissue structures of the patient as the patient is positioned in an operating environment which may be characterized by a global coordinate system; a computing system operatively coupled to the elongate instrument and configured to present a control interface to an operator; and two or more magnetic field sources, each of which is controllably movably coupled relative to the patient by a robotic coupling assembly operatively coupled to the computing system; wherein in response to commands provided by the operator to the computing system, the computing system is configured to precisely navigate a portion of the elongate body of the elongate instrument relative to the one or more tissue structures of the patient by causing each of the two or more magnetic field sources to be positioned and oriented relative to the patient and relative to the two or more magnetic steering nodes of the elongate instrument using each of the robotic coupling assemblies. The computing system may be configured to control magnetic field emissions from the two or more magnetic field sources. The computing system may be configured to control magnetic field emissions from the two or more magnetic field sources independently. At least one robotic coupling assembly may comprise a robotic arm. The robotic arm may comprise one or more electromechanically controllable degrees of freedom. The system further may comprise an electromechanical insertion/retraction interface configured to controllably cause the elongate body to controllably insert or retract relative to the patient. The electromechanical insertion/retraction interface may comprise one or more engagement wheels operably coupled between one or more drive motors and an outer aspect of the elongate body of the elongate instrument. The system further may comprise an electromechanical roll interface configured to controllably cause the elongate body to controllably roll relative to the patient. The electromechanical roll interface may comprise one or more engagement wheels operably coupled between one or more drive motors and an outer aspect of the elongate body of the elongate instrument. The system further may comprise one or more ultrasound transducers operatively coupled to the computing system and configured to capture images pertaining to the location of one or more of the magnetic steering nodes relative to the one or more tissue structures of the patient. At least one of the one or more ultrasound transducers may be coupled to at least one of the magnetic field sources. The system further may comprise one or more image capture devices operatively coupled to the computing system and configured to provide image information pertinent to the position or orientation of at least one of the magnetic field sources. The elongate instrument may comprise one or more encoded portions configured such that they may be electronically observed by an operatively coupled encoder reader to determine a position or orientation of an encoded portion relative to the encoder reader. At least one of the one or more encoded portions may be positioned longitudinally relative to the elongate instrument. At least one of the one or more encoded portions may be positioned radially relative to the elongate instrument. The computing system may be operatively coupled to a fluoroscopy system configured to provide image information pertaining to the elongate instrument and adjacent tissue structures of the patient. The computing system may be operatively coupled to a radiography system configured to provide image information pertaining to the elongate instrument and adjacent tissue structures of the patient. The system further may comprise one or more electromagnetic localization sensors coupled to one or more locations along the elongate instrument, the sensors operatively coupled with a localization transmitter operatively coupled to the computing system and configured to determine localization information pertaining to the one or more electromagnetic localization sensors relative to transmissions from the localization transmitter. The one or more localization sensors may comprise one or more receiving coils operatively coupled to the localization transmitter and computing system and configured for determining position and orientation of the one or more localization sensors in three dimensional space relative to the localization transmitter. The system may comprise two magnetic steering nodes and two magnetic field sources. Each of the magnetic field sources may be primarily directed to one of the two magnetic steering nodes. The computing system may be configured to control shape and position of the elongate instrument in view of both magnetic field sources, each of which may influence the position of each of the magnetic steering nodes due to overlap patterns of each of the magnetic field sources. Each of the two magnetic steering nodes may be positioned discretely apart such that each of the magnetic field sources may be positioned and oriented by each of the robotic coupling assemblies to be only substantially influential upon one of the two magnetic steering nodes. At least one of the two or more magnetic steering nodes may comprise a ferromagnetic material. The ferromagnetic material may comprise iron, steel, and iron alloys. At least one of the two or more magnetic steering nodes may comprise a paramagnetic material. The paramagnetic material may comprise stainless steel, platinum, aluminum, magnesium, and lithium. At least one of the two or more magnetic steering nodes may comprise a ring geometry with an aperture defined therethrough. At least one of the two or more magnetic steering nodes may comprise a radially homogeneous construct. At least one of the two or more magnetic steering nodes may comprise a circumferentially homogeneous construct. At least one of the two or more magnetic steering nodes may comprise a radially non-homogeneous portion. At least one of the two or more magnetic steering nodes may comprise a circumferentially non-homogeneous portion. At least one of the two or more magnetic field sources may comprise a permanent magnet. The permanent magnet may comprise a material selected from the list consisting of: iron, iron alloy, steel, nickel, cobalt, neodymium, and samarium. The permanent magnet may be selected to have a field shape geometry. The field shape geometry may be selected from the group consisting of: wide, narrow, and irregular. The field shape geometry may be configured to be discrete relative to an outer shape of the permanent magnet. At least one of the two or more magnetic field sources may comprise an electromagnet coil circuit. The electromagnet coil circuit may comprise copper or gold. The electromagnet coil circuit may comprise a substantially circular coil pattern. At least one of the two or more magnetic steering nodes may comprise a controllably adjustable amount of magnetism. At least one of the two or more magnetic steering nodes may be controllably adjusted in longitudinal position relative to the elongate body of the elongate instrument. The elongate instrument may comprise an engagement portion selected from the group consisting of: a resonant member assembly; a radiation emission assembly; an open interior cannulation volume; a controlled vacuum lumen; a fluid perfusion lumen; a flow redirection portion; a controllably actuated scissor tool; a controllably actuated grasper tool; a capture/agitation mesh interface; a retractable loose capture forked structure; and a helical thrombectomy assembly. The computing system may comprise a neural network informed by preoperative information pertaining to the patient, operational selections made by the operator through the control interface preoperatively, and operational selections made by the operator intraoperatively through the control interface. The preoperative information pertaining to the patient may be selected from the group consisting of: two-dimensional image data pertaining to the tissue structures of the patient; three-dimensional image data pertaining to the tissue structures of the patient; information pertaining to prior interventions; locations and geometries of potential hazards within the patient anatomy. The operational selections made by the operator through the control interface preoperatively may comprise a provisional access pathway to a targeted tissue structure. The provisional access pathway may be generated at least in part using automated calculations from the computing system based at least in part upon the preoperative information pertaining to the patient and operational selections made by the operator through the control interface preoperatively. The control interface to the operator may be operated at least in part via natural language commands from the operator interpreted by the computing system. The neural network may comprise a reinforcement learning model informed by one or more agents configured to provide operational instructions to operate the elongate instrument relative to the patient via control of the operatively coupled two or more magnetic steering nodes, two or more magnetic field sources, and associated robotic coupling assemblies in view of the preoperative information pertaining to the patient, operational selections made by the operator through the control interface preoperatively, and operational selections made by the operator intraoperatively through the control interface. The neural network may comprise a multi-agent model configured to parallel-process operation of the elongate instrument relative to the patient in view of a plurality of reward and priority configurations pertinent to a plurality of agents comprising the multi-agent model. The neural network also may be configured to be multi-modal in operation, in that it is configured to have a plurality of modes of operating the elongate instrument relative to the patient in a time domain. The plurality of modes may comprise an insertion navigation mode relative to tissue structures of the patient and a controlled mechanical stimulation mode relative to tissue structures of the patient. The controlled mechanical stimulation mode may be configured to provide controlled oscillatory motion of a portion of the elongate instrument relative to a targeted tissue structure. The elongate instrument may be deployed within the vasculature of the patient, and wherein controlled oscillatory motion of a portion of the elongate instrument relative to a targeted tissue structure comprises oscillatory motion selected to disrupt a thrombus or clot structure positioned within the vasculature for removal in a potential cardiovascular stroke paradigm.

Another embodiment is directed to an elongate instrument navigation method for conducting a medical procedure on a patient, comprising: providing an elongate instrument comprising an elongate body coupled to two or more discretely positioned magnetic steering nodes, the elongate instrument comprising an insertion length selected such that a distal portion may be inserted to reach a position of a targeted interventional structure within the body of the patient, and a cross-sectional size profile configured to be navigated through one or more tissue structures of the patient as the patient is positioned in an operating environment which may be characterized by a global coordinate system; providing a computing system operatively coupled to the elongate instrument and configured to present a control interface to an operator; and providing two or more magnetic field sources, each of which is controllably movably coupled relative to the patient by a robotic coupling assembly operatively coupled to the computing system; wherein in response to commands provided by the operator to the computing system, the computing system is configured to precisely navigate a portion of the elongate body of the elongate instrument relative to the one or more tissue structures of the patient by causing each of the two or more magnetic field sources to be positioned and oriented relative to the patient and relative to the two or more magnetic steering nodes of the elongate instrument using each of the robotic coupling assemblies. The computing system may be configured to control magnetic field emissions from the two or more magnetic field sources. The computing system may be configured to control magnetic field emissions from the two or more magnetic field sources independently. At least one robotic coupling assembly may comprise a robotic arm. The robotic arm may comprise one or more electromechanically controllable degrees of freedom. The method further may comprise providing an electromechanical insertion/retraction interface configured to controllably cause the elongate body to controllably insert or retract relative to the patient. The electromechanical insertion/retraction interface may comprise one or more engagement wheels operably coupled between one or more drive motors and an outer aspect of the elongate body of the elongate instrument. The method further may comprise providing an electromechanical roll interface configured to controllably cause the elongate body to controllably roll relative to the patient. The electromechanical roll interface may comprise one or more engagement wheels operably coupled between one or more drive motors and an outer aspect of the elongate body of the elongate instrument. The method further may comprise providing one or more ultrasound transducers operatively coupled to the computing system and configured to capture images pertaining to the location of one or more of the magnetic steering nodes relative to the one or more tissue structures of the patient. At least one of the one or more ultrasound transducers may be coupled to at least one of the magnetic field sources. The method further may comprise providing one or more image capture devices operatively coupled to the computing system and configured to provide image information pertinent to the position or orientation of at least one of the magnetic field sources. The elongate instrument may comprise one or more encoded portions configured such that they may be electronically observed by an operatively coupled encoder reader to determine a position or orientation of an encoded portion relative to the encoder reader. At least one of the one or more encoded portions may be positioned longitudinally relative to the elongate instrument. At least one of the one or more encoded portions may be positioned radially relative to the elongate instrument. The computing system may be operatively coupled to a fluoroscopy system configured to provide image information pertaining to the elongate instrument and adjacent tissue structures of the patient. The computing system may be operatively coupled to a radiography system configured to provide image information pertaining to the elongate instrument and adjacent tissue structures of the patient. The method further may comprise providing one or more electromagnetic localization sensors coupled to one or more locations along the elongate instrument, the sensors operatively coupled with a localization transmitter operatively coupled to the computing system and configured to determine localization information pertaining to the one or more electromagnetic localization sensors relative to transmissions from the localization transmitter. The one or more localization sensors may comprise one or more receiving coils operatively coupled to the localization transmitter and computing system and configured for determining position and orientation of the one or more localization sensors in three dimensional space relative to the localization transmitter. The method may comprise two magnetic steering nodes and two magnetic field sources. Each of the magnetic field sources may be primarily directed to one of the two magnetic steering nodes. The computing system may be configured to control shape and position of the elongate instrument in view of both magnetic field sources, each of which may influence the position of each of the magnetic steering nodes due to overlap patterns of each of the magnetic field sources. Each of the two magnetic steering nodes may be positioned discretely apart such that each of the magnetic field sources may be positioned and oriented by each of the robotic coupling assemblies to be only substantially influential upon one of the two magnetic steering nodes. At least one of the two or more magnetic steering nodes may comprise a ferromagnetic material. The ferromagnetic material may comprise iron, steel, and iron alloys. At least one of the two or more magnetic steering nodes may comprise a paramagnetic material. The paramagnetic material may comprise stainless steel, platinum, aluminum, magnesium, and lithium. At least one of the two or more magnetic steering nodes may comprise a ring geometry with an aperture defined therethrough. At least one of the two or more magnetic steering nodes may comprise a radially homogeneous construct. At least one of the two or more magnetic steering nodes may comprise a circumferentially homogeneous construct. At least one of the two or more magnetic steering nodes may comprise a radially non-homogeneous portion. At least one of the two or more magnetic steering nodes may comprise a circumferentially non-homogeneous portion. At least one of the two or more magnetic field sources may comprise a permanent magnet. The permanent magnet may comprise a material selected from the list consisting of: iron, iron alloy, steel, nickel, cobalt, neodymium, and samarium. The permanent magnet may be selected to have a field shape geometry. The field shape geometry may be selected from the group consisting of: wide, narrow, and irregular. The field shape geometry may be configured to be discrete relative to an outer shape of the permanent magnet. At least one of the two or more magnetic field sources may comprise an electromagnet coil circuit. The electromagnet coil circuit may comprise copper or gold. The electromagnet coil circuit may comprise a substantially circular coil pattern. At least one of the two or more magnetic steering nodes may comprise a controllably adjustable amount of magnetism. At least one of the two or more magnetic steering nodes may be controllably adjusted in longitudinal position relative to the elongate body of the elongate instrument. The elongate instrument may comprise an engagement portion selected from the group consisting of: a resonant member assembly; a radiation emission assembly; an open interior cannulation volume; a controlled vacuum lumen; a fluid perfusion lumen; a flow redirection portion; a controllably actuated scissor tool; a controllably actuated grasper tool; a capture/agitation mesh interface; a retractable loose capture forked structure; and a helical thrombectomy assembly. The computing system may comprise a neural network informed by preoperative information pertaining to the patient, operational selections made by the operator through the control interface preoperatively, and operational selections made by the operator intraoperatively through the control interface. The preoperative information pertaining to the patient may be selected from the group consisting of: two-dimensional image data pertaining to the tissue structures of the patient; three-dimensional image data pertaining to the tissue structures of the patient; information pertaining to prior interventions; locations and geometries of potential hazards within the patient anatomy. The operational selections made by the operator through the control interface preoperatively may comprise a provisional access pathway to a targeted tissue structure. The provisional access pathway may be generated at least in part using automated calculations from the computing system based at least in part upon the preoperative information pertaining to the patient and operational selections made by the operator through the control interface preoperatively. The control interface to the operator may be operated at least in part via natural language commands from the operator interpreted by the computing system. The neural network may comprise a reinforcement learning model informed by one or more agents configured to provide operational instructions to operate the elongate instrument relative to the patient via control of the operatively coupled two or more magnetic steering nodes, two or more magnetic field sources, and associated robotic coupling assemblies in view of the preoperative information pertaining to the patient, operational selections made by the operator through the control interface preoperatively, and operational selections made by the operator intraoperatively through the control interface. The neural network may comprise a multi-agent model configured to parallel-process operation of the elongate instrument relative to the patient in view of a plurality of reward and priority configurations pertinent to a plurality of agents comprising the multi-agent model. The neural network also may be configured to be multi-modal in operation, in that it is configured to have a plurality of modes of operating the elongate instrument relative to the patient in a time domain. The plurality of modes may comprise an insertion navigation mode relative to tissue structures of the patient and a controlled mechanical stimulation mode relative to tissue structures of the patient. The controlled mechanical stimulation mode may be configured to provide controlled oscillatory motion of a portion of the elongate instrument relative to a targeted tissue structure. The elongate instrument may be deployed within the vasculature of the patient, and wherein controlled oscillatory motion of a portion of the elongate instrument relative to a targeted tissue structure comprises oscillatory motion selected to disrupt a thrombus or clot structure positioned within the vasculature for removal in a potential cardiovascular stroke paradigm.

### DETAILED DESCRIPTION

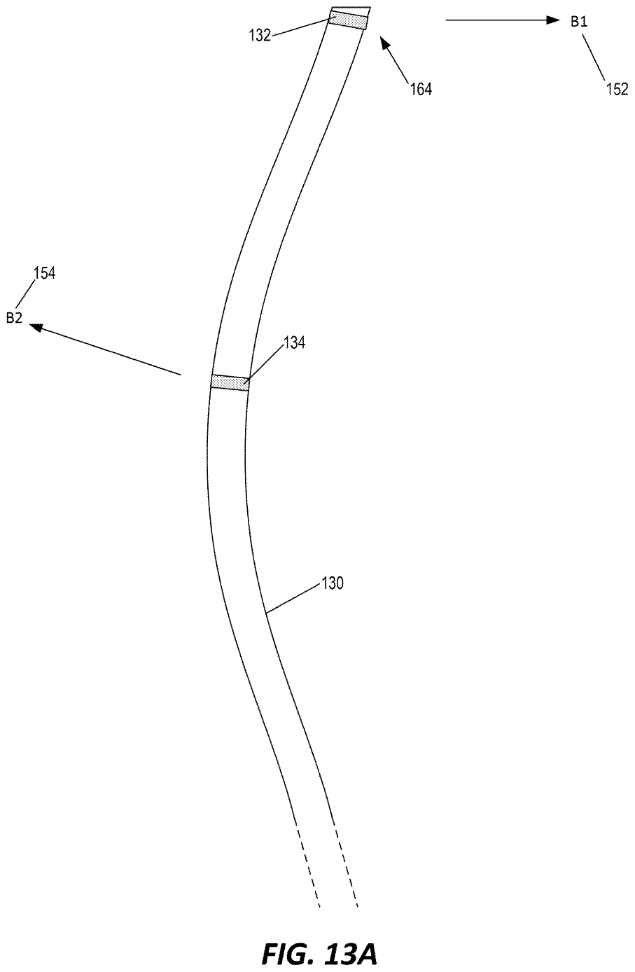

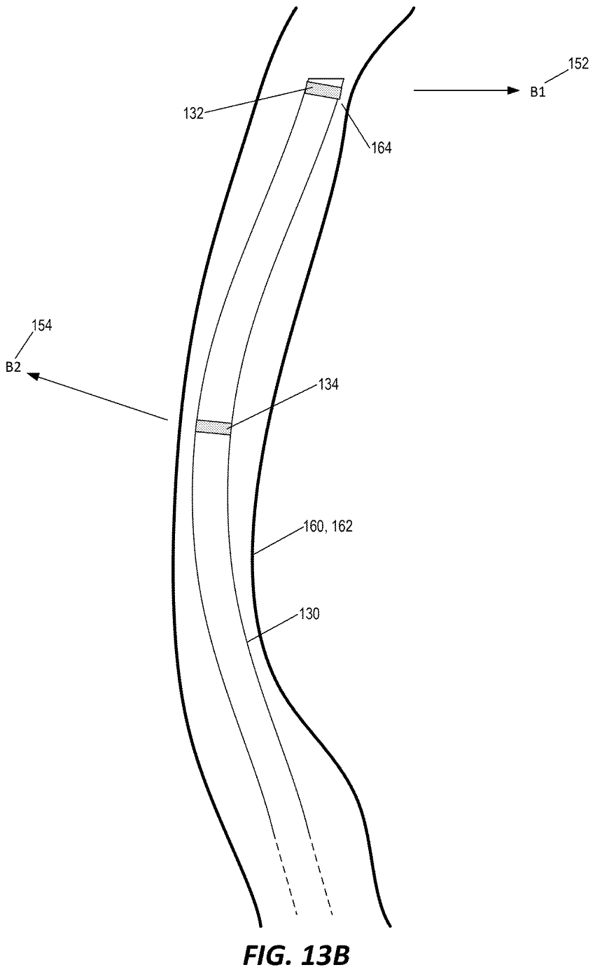

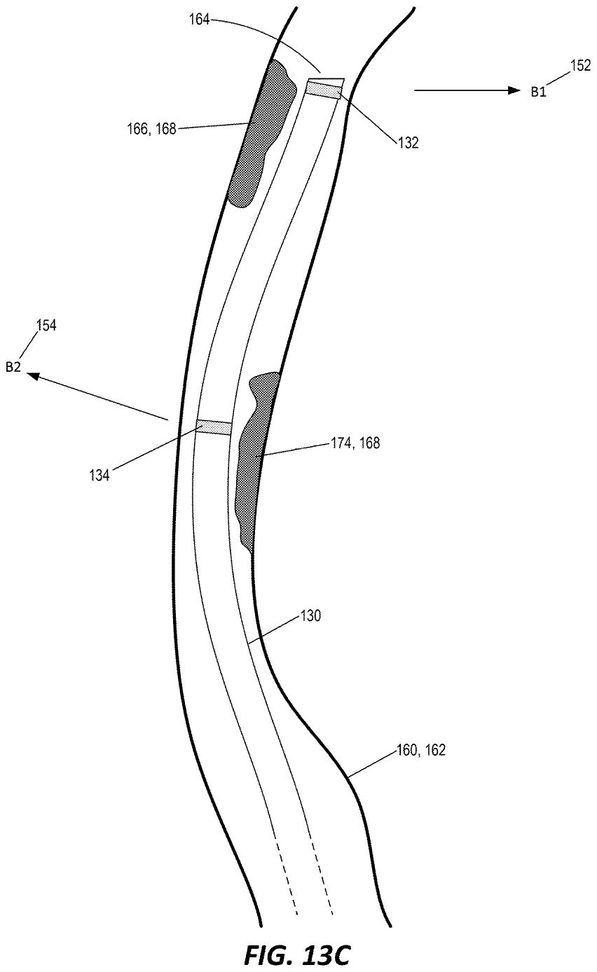

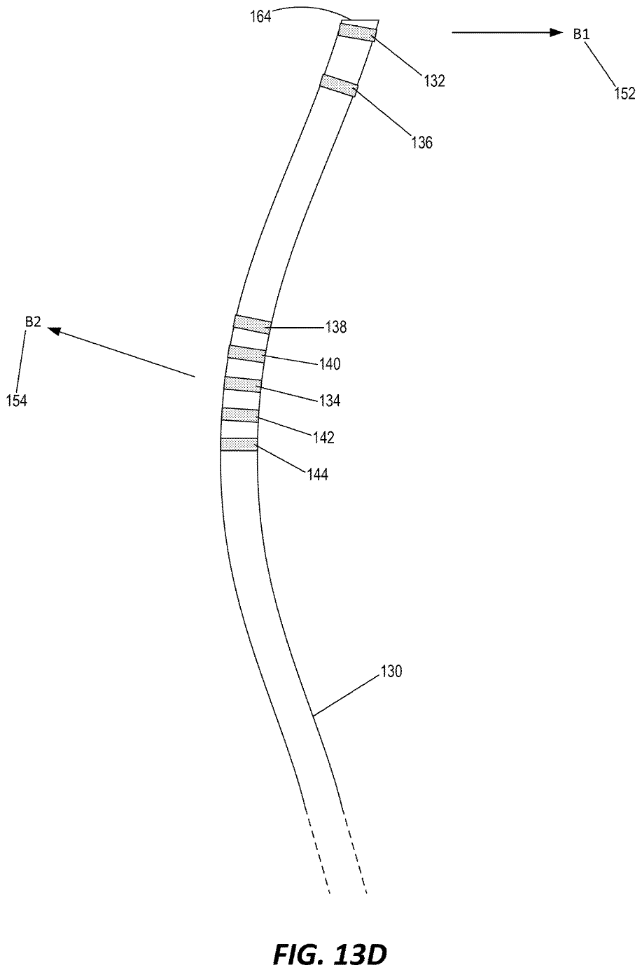

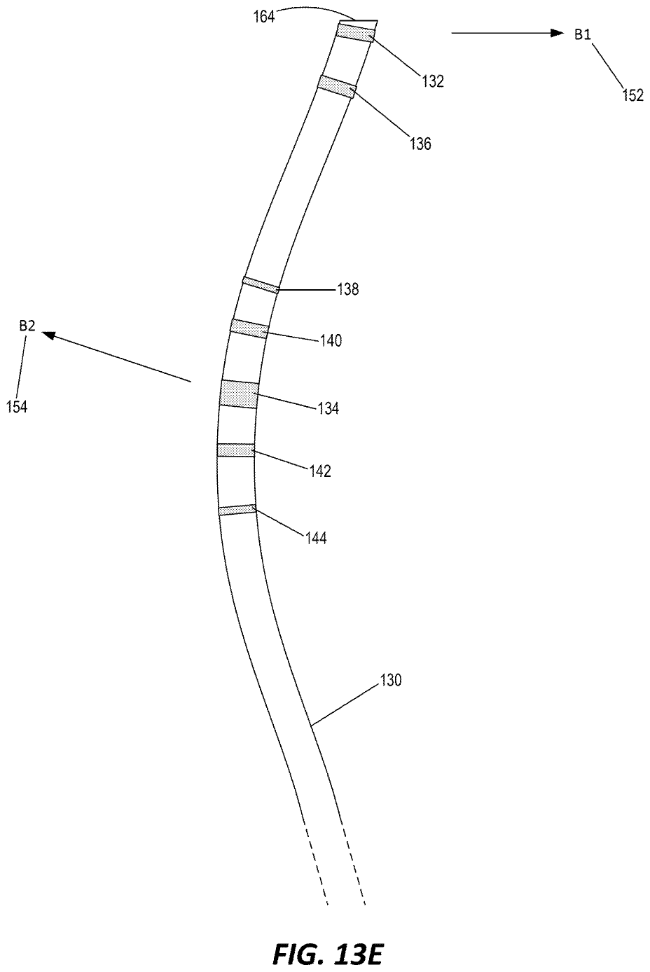

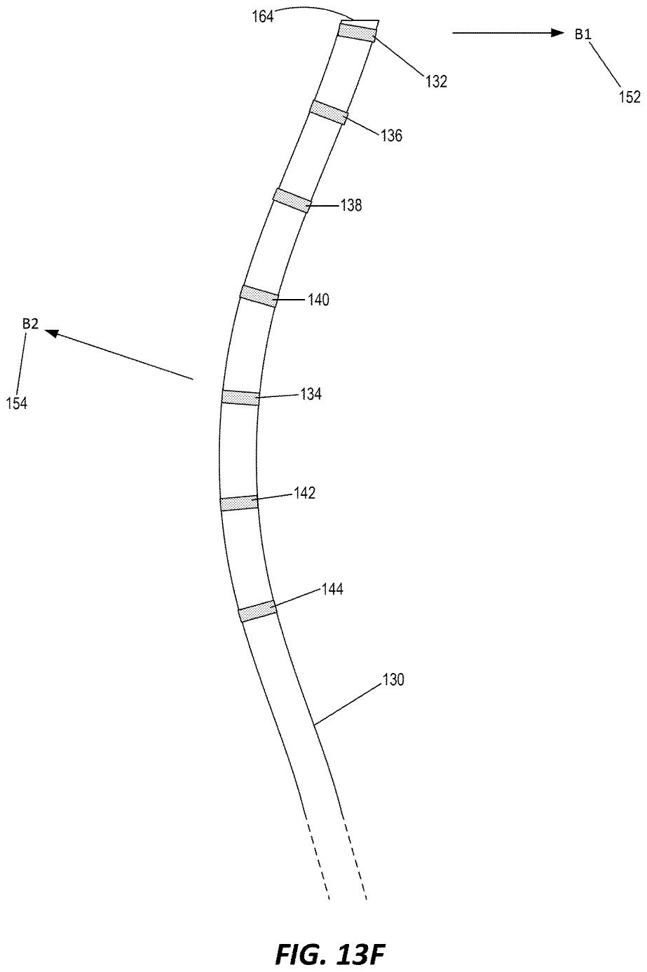

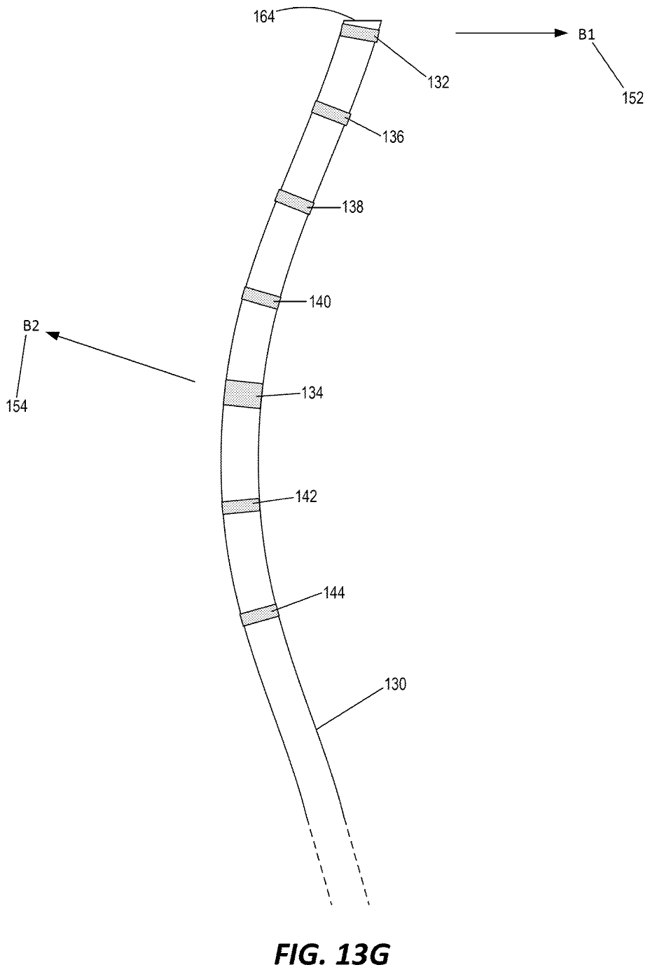

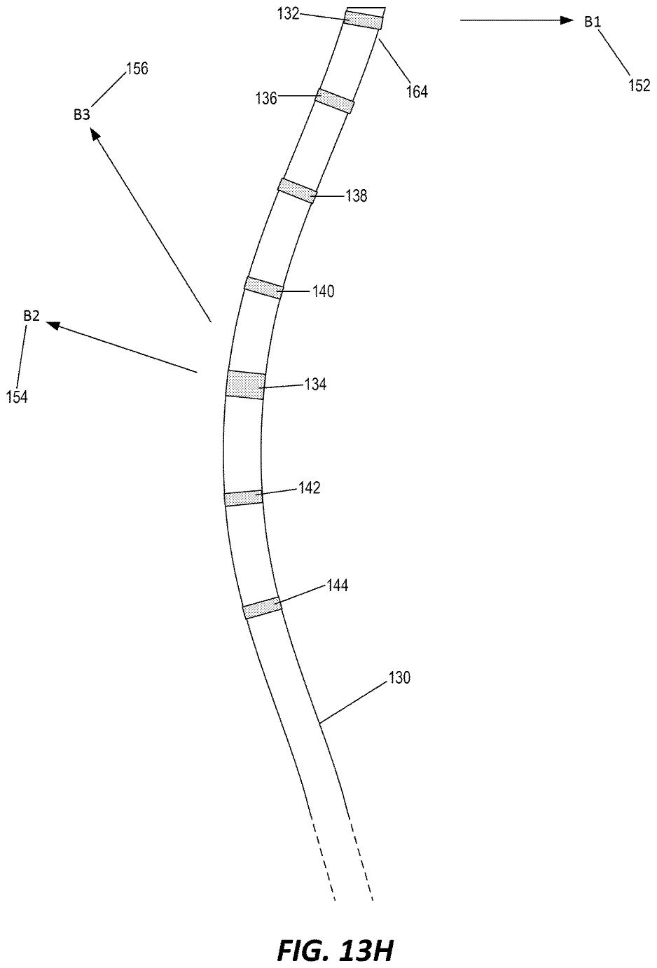

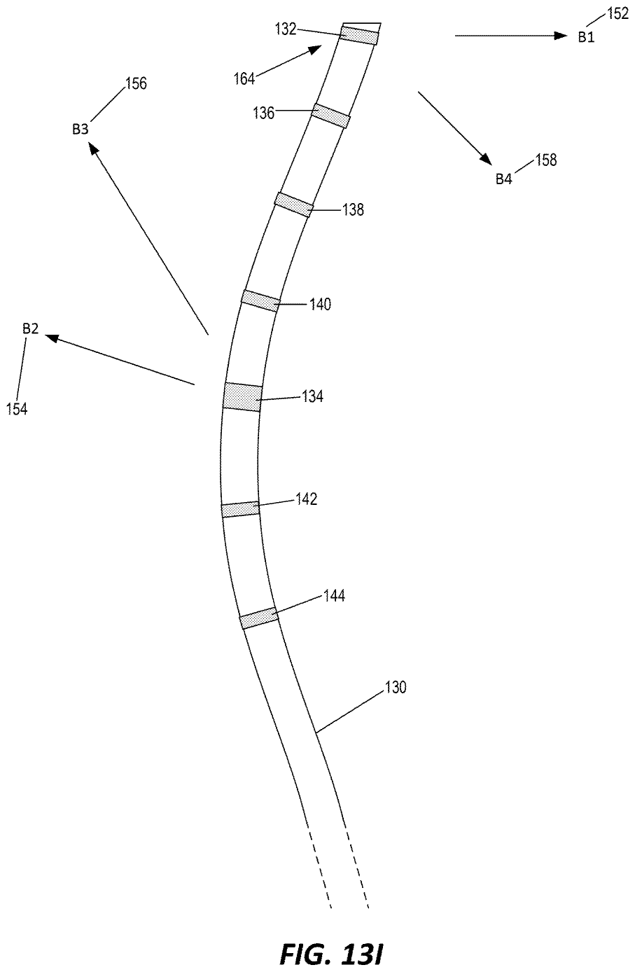

Referring to FIG. 13A, a flexible instrument (130) such as a catheter (for example, may comprise a tubular geometry and/or substantially round cross sectional profile) is illustrated having a plurality of ferromagnetic steerability nodes (132, 134) configured to impart steering loads upon the instrument (130) in accordance with one or more applied magnetic fields (B1, 152; B2, 154) which may or may not be functionally intersecting. These discrete nodes may comprise ferromagnetic materials (such as iron, iron alloys, steel), paramagnetic, or lightly magnetic, materials (such as stainless steel, platinum, aluminum, magnesium, and lithium), or diamagnetic materials (such as copper or gold), depending upon the particular scenario, associated tissue structures, and desired guidance loads, for example. For example, generally two or more magnetic fields or sources of so-called “magnetic flux” may be superimposed upon each other such that a hybrid functional magnetic field may be experienced from a steering or navigation loading perspective at a given steerability node (132, 134). When magnetic fields have a high degree of geometric focus in a focal region or volume, and associated functional fall-off with greater spatial separation from the focal volume, they generally will not be as impactful with regard to steering/navigation at distances of relatively large separation from the focal volume. In other words, referring back to FIG. 13A, in a configuration wherein applied and vectored magnetic fields B1 (152) and B2 (154) have relatively tight geometric focal volumes centered around each of the respective targeted steerability nodes (132 near distal end 164 of the instrument 130, 134 positioned more proximally), the instrument (130) steering impact of B1 (152) on steerability node 132 will be substantially discrete relative to the instrument (130) steering impact of B2 (154) on steerability node 134. Conversely, should the the applied and vectored magnetic fields B1 (152) and B2 (154) have relatively wide functional focal volumes relative to the geometries of the instrument, first steerability node (132), and second steerability node (134), the steering of each of these nodes (132, 134) may be impacted by each of the applied and vectored magnetic fields B1 (152) and B2 (154). An associated computing system may be configured to account for the focal volumes/geometries of each of the applied and vectored magnetic fields (in FIG. 13A, B1 152, and B2 154) relative to the geometries, positions, orientations, and materials of the instrument (130) and steerability nodes (132, 134) to produce a broad range of multi-nodal steerability functionality for the instrument (130) relative to surrounding tissue structures.

Thus referring to FIG. 13B, an instrument (130) similar to that shown in FIG. 13A is illustrated navigating through an anatomic lumen (160) such as a blood vessel (162) with the assistance of applied and vectored magnetic fields B1 and B2 (152, 154). Depending upon the medical and/or operational challenges and/or priorities, it may be desirable to avoid or minimize contact between certain portions of the lumen (160) or blood vessel (162) and instrument (130). For example, referring to FIG. 13C, a plurality of endoluminal objects (166, 174) which may, for example, comprise endoluminal plaques (168) or other potentially vulnerable structures (implanted objects such as stents or stent grafts, zones of potential weakness or aneurysm, locations of prior surgery or intervention, etc), may be intentionally avoided in terms of contact with the instrument (130) during navigation of the instrument through the lumen (160).

Referring to FIGS. 13D-13G, different configurations, sizes, locations, orientations, and pluralities of ferromagnetic steerability nodes or elements (132, 134, 136, 138, 140, 142, 144) may be selected to accommodate various sophisticated steering impacts upon the instrument (130) from applied and vectored magnetic fields such as B1 (152) and B2 (154), dependent upon the magnetic flux configurations (focal patterns and proximity, as well as vectoring, for example) of the fields B1 (152) and B2 (154) as noted above.

Referring to FIG. 13D, magnetic fields such as B1 (152) and B2 (154) are shown applied and vectored to an instrument (130) configuration comprising two more distally-positioned steerability elements (132, 136) as well as five more proximally positioned elements (138, 140, 134, 142, 144; each approximately of the same volume and material in this example) to provide for steering and navigation control. Referring to FIG. 13E, a configuration somewhat similar to that of FIG. 13D is illustrated, with configuration of FIG. 13E comprising two distally-positioned steerability elements (132, 136) as well as five more proximally positioned elements (138, 140, 134, 142, 144 with greater spacing relative to the configuration of FIG. 13D; also the volume of element 134 is greater, to have a greater magnetic field based steering load at such location relative to an equivalently positioned/oriented element of smaller ferromagnetic material). In various embodiments, it may be desirable to not only have material volume differences for varied control handles, but also certain specialized configurations such as those which may be radially nonhomogeneous (i.e., comprising different materials at different radial positions relative to a central axis, such as a central axis through an aperture formed in a ring-like steerability element; for example, one material on the inside aspect of the ring-like steerability element structure, another material on the outer aspects of the ring-like steerability element structure) and/or circumferentially nonhomogeneous (such as in a configuration wherein at least one portion of a ring-like steerability element has a lump of greater volume at one circumferential position, or perhaps a lump or portion of different material composite to the other material). FIG. 13F illustrates a instrument (130) configuration wherein seven substantially similar steerability elements (132, 136, 138, 140, 134, 142, 144) are positioned along the distal length of the instrument (130) with approximately equidistant spacing, all subject to the applied and vectored magnetic fields shown (B1, 152; B2, 154) for applied steering/navigation loading. FIG. 13G illustrates a configuration similar to that of FIG. 13F, but with one steerability element (134) comprising larger geometry (to have a greater magnetic field based steering load at such location relative to an equivalently positioned/oriented element of smaller ferromagnetic material) to illustrate that many variations may be desired and utilized.

As noted above, magnetic fields or magnetic flux patterns may be superimposed over each other to provide complex and hybrid loading patterns upon subject ferromagnetic elements. Referring to FIG. 13H, to illustrate such a configuration, three fields (B1, 152; B2, 154; B3, 156) are applied and vectored relative to an instrument (130) such as the one featured in FIG. 13G; FIG. 13I illustrates a similar configuration with four magnetic fields (B1, 152; B2, 154; B3, 156; B4, 158) are applied and vectored relative to an instrument (130) such as the one featured in FIG. 13G. Precision steering an instrument such as that of any of FIGS. 13A-13I via the utilization of vectored and applied fields such as those illustrated as B1, B2, B3, and/or B4, for example (elements 152, 154, 156, and/or 158) can require a significant amount of computational complexity as regards application and vectoring of such field(s).

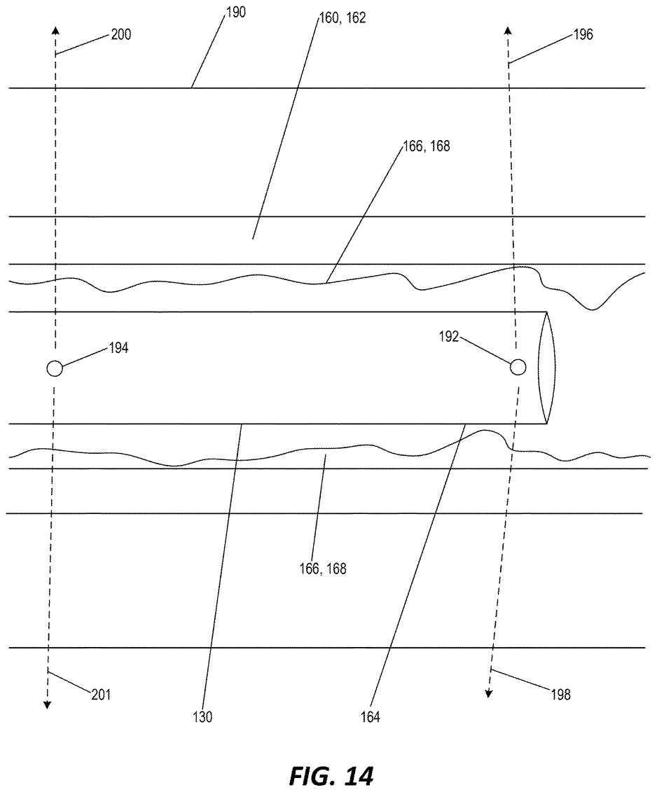

Referring to FIG. 14, as noted above, in some applications it may be desirable to navigate an instrument (130) through tissue structures such as naturally formed lumens (160) such as blood vessels (162) of a human, and these lumens or vessels (160, 162) may present physical obstacles or objects (166), such as plaques (168), stents, or other objects which may be desirably avoided, for example. FIG. 14 illustrates a distal tip steering node (192) being physically influenced or loaded (196, 198), such as via two different applied and vectored magnetic fields, while more proximally, another steering node (194) is being simultaneously physically influenced or loaded (200, 201), by two applied and vectored magnetic fields which are vectored and applied in approximately equal and opposite vectoring manners, such as to assist in temporarily maintaining a position or orientation of that portion (194) of the instrument (130) relative to the associated tissue (160, 162) structures or other objects (166, 168).

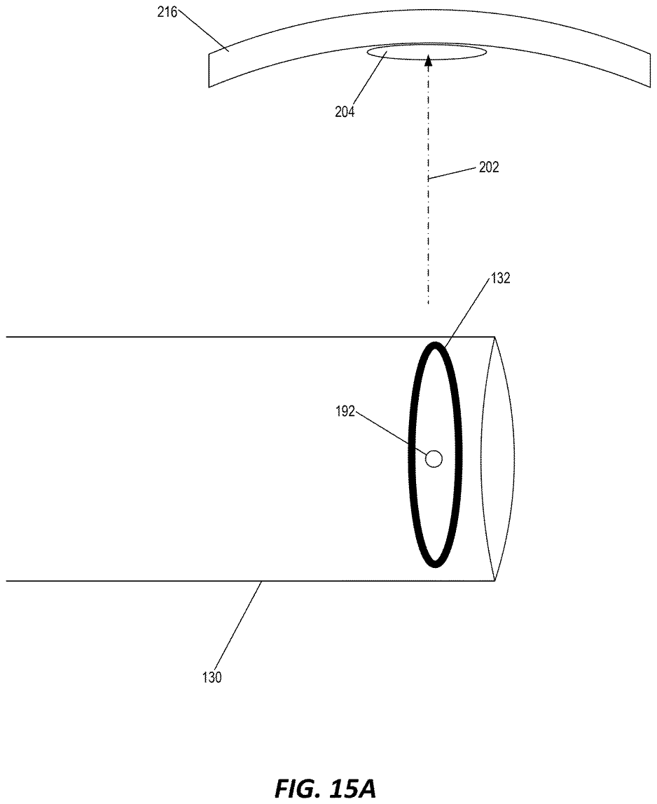

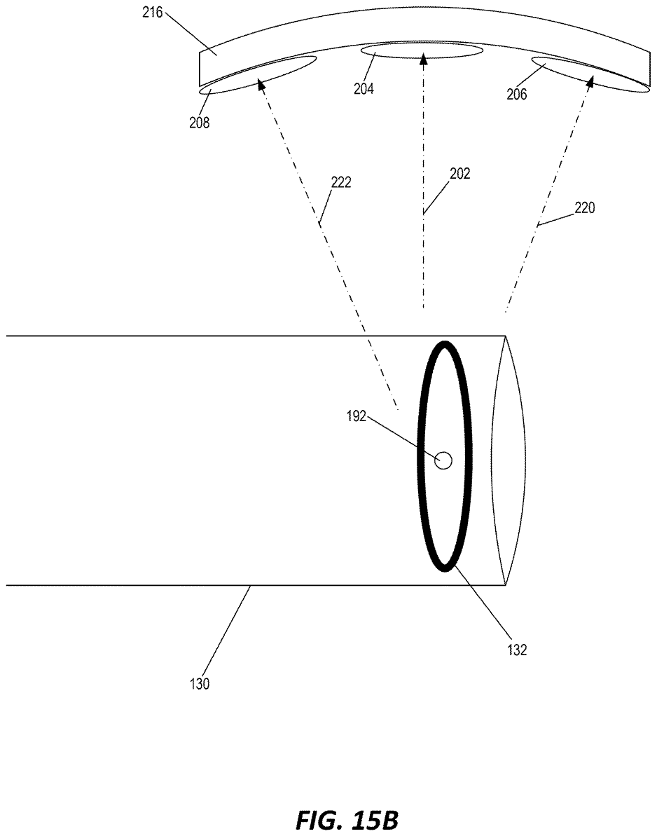

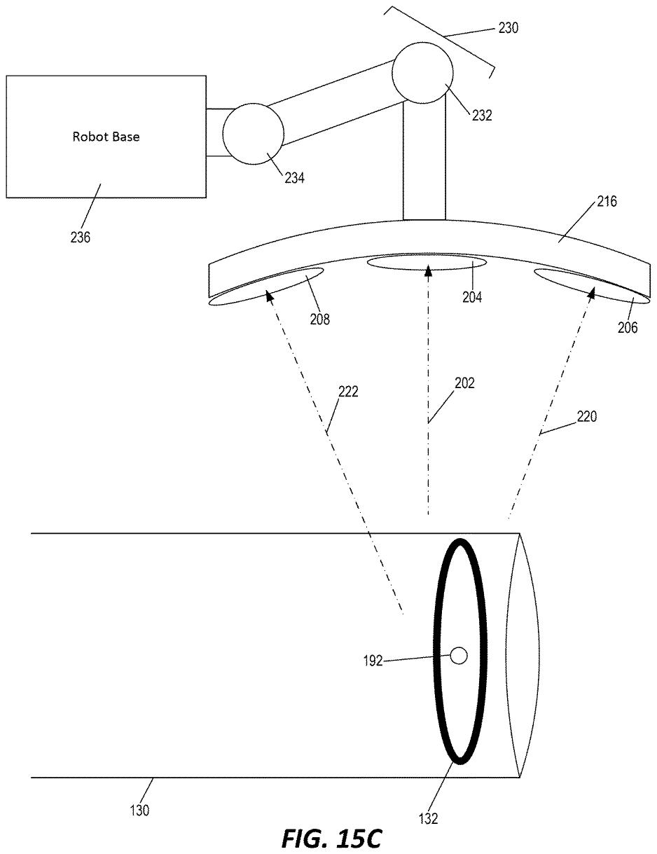

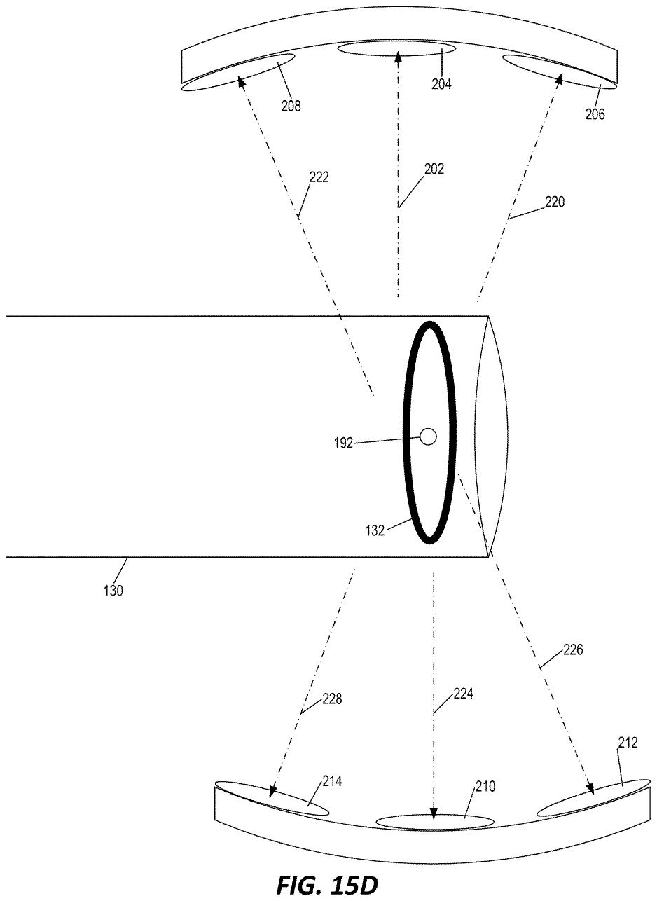

Referring to FIG. 15A, an elongate instrument (130) is illustrated with a ring-shaped ferromagnetic element (132) which is within proximity of steering loading influence by an applied and vectored (202) magnetic field developed by a magnetic element (204), such as a permanent magnet or electromagnet (i.e., which may comprise a coil or series thereof configured to produce magnetic flux when electrical current is circulated therethrough), which may be coupled to a mounting structure (216) such as the arcuate member shown which may comprise a rigid support structure configured to stably and movably position and orient the magnetic element (204) relative to the ferromagnetic element (132) and/or effective steering node (192) to influence steering of the elongate instrument (130). The effective steering vector (202) of the illustrated magnetic element (204) relative to the ferromagnetic element (132) may be adjusted by moving (i.e., repositioning and/or reorienting) the mounting structure (216), for example. Referring to FIG. 15B, alternatively, a plurality of magnetic elements (204, 206, 208) may be utilized to provide a plurality of magnetic field vectors (202, 222, 224, respectively) in an alternated and/or simultaneous fashion to provide desired steering and positioning influence to the targeted magnetic element (132) and steering node (192) of the elongate instrument (130). As noted above, applied and vectored fields may be superimposed, such that, for example, a relatively large upward (i.e., generally in the directed of depicted vector 202) steering load may be applied with similar magnetic flux being developed by each of the three magnetic elements (204, 206, 208) applied at the depicted vectors (202, 222, 224, respectively), with components orthogonal to vector 202 generally cancelling each other in the case wherein vectors 220 and 222 generally have equal and opposite orthogonal components and the fields from elements 206 and 208 are of equivalent specification. A configuration such as that of FIG. 15B may be positioned and oriented relatively stably or in a fixed manner relative to the instrument (130) during a given portion of an operation, with steering influence changing in such time window based upon the application and vectoring variability provided by the plurality of magnetic elements (204, 206, 208). Alternatively, one or more elements may be repositioned and/or reoriented, such as via collective repositioning and/or reorientation provided by moving the magnetic element mounting structure (216) fixedly coupled to each element (204, 206, 208) in FIG. 15B.

Referring to FIG. 15C, in one embodiment a magnetic element mounting structure (216) such as that illustrated in FIG. 15A or 15B may be operatively coupled using a controllably movable coupling assembly or manipulator (230), such as an electromechanical robotic arm assembly or robotic coupling assembly which may comprise one or more controllably movable joints (232, 234) and be coupled to a robotic arm base (236) or other structure within a global coordinate system. Such a configuration may be configured to controllably and automatically reposition and/or reorient the magnetic element mounting structure (216) relative to the elongate instrument (130) and associated steering elements (such as 132 of FIG. 15C).

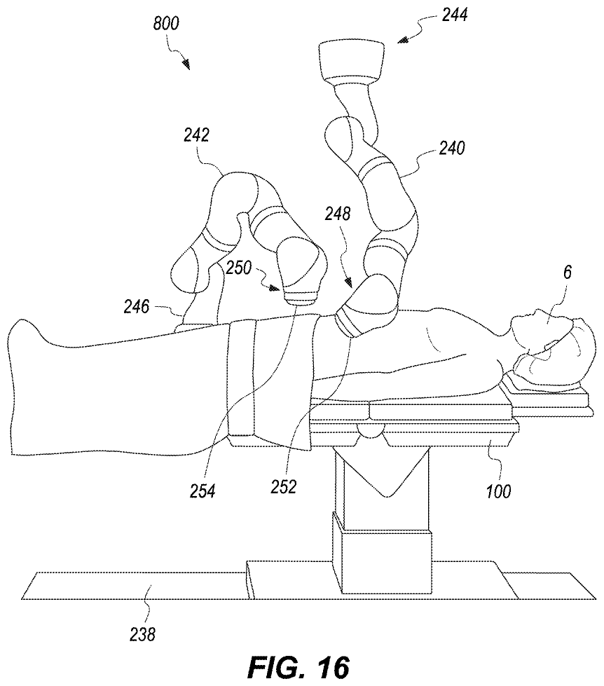

Referring to FIG. 15D, a plurality of magnetic element mounting structures (216, 218) may be utilized to present and control a plurality of magnetic steering elements (204, 206, 208; 224, 226, 228) as vectors relative to a given steering node (192) or steering element (132) as shown (vectors 202, 220, 222 shown for upper element mounting structure 216; vectors 224, 226, 228 shown for lower element mounting structure 218). One of more of the mounting structures (216, 218) may be controllably movable relative to associated tissue structures and/or elongate instrument (130) as with the configuration of FIG. 15C, such as via two or more controllably movable coupling assemblies, such as two or more robotic arms (240, 242), as shown in FIG. 16, for example. Each of the magnetic steering elements or magnetic field sources in the various configurations described herein may comprise permanent magnets (comprising materials such as iron, iron alloy, steel, nickel, cobalt, neodymium, and/or samarium) or electromagnet coil circuits (comprising, for example, a substantially circular coil pattern of conductive material such as copper or gold), with field shapes which may be naturally-occurring given the magnetic field source geometry or configuration, or adjusted or modified via field shunt or other control structure, such that the associated field shape geometry preferably is known (for example, may be wide in shape, narrow in shape, notched or irregular in shape, and/or generally discrete given the outer shape of the permanent magnet or electromagnetic coil circuit. Field strength may be adjusted electronically with electromagnetic coil circuit configurations (lower current being associated with lower field strength), aggregation (for example, with more than one field overlapping to provide additional magnetic flux and applied load, such as in FIG. 15B), and also with proximity and/or orientation in view of field shape/geometry. Further, as noted above, paramagnetic materials also may be incorporated into magnetic field sources and/or steerability nodes, comprising lightly-magnetic materials such as stainless steel, platinum, aluminum, magnesium, and lithium.

Referring to FIG. 16, a system configuration (800) is illustrated wherein a patient (6) is shown on an operating table (100), the operating table (100) coupled to the floor (238) or other structure of an operating room; two robotic arms (240, 242) are illustrated which may be coupled at their proximal ends (244, 246, respectively) to other structures such as robotic base assemblies and/or a portion of the operating environment such as a floor (238), movable gantry member (which may be suspended from an operating environment ceiling, for example, and which may be configured to be movable, such as controllably, automatically, and/or electromechanically movable, in one mode and lockable into a fixed configuration in another mode), or other structure within the operating environment. The robotic arms may be systems available from suppliers such as Kuka™ or Universal Robotics™, and may have distal ends (248, 250, respectively) that are coupleable to end effector assemblies (252, 254, respectively) configured to assist in various steerability, navigation, imaging, and/or otherwise interventional aspects, as described below.

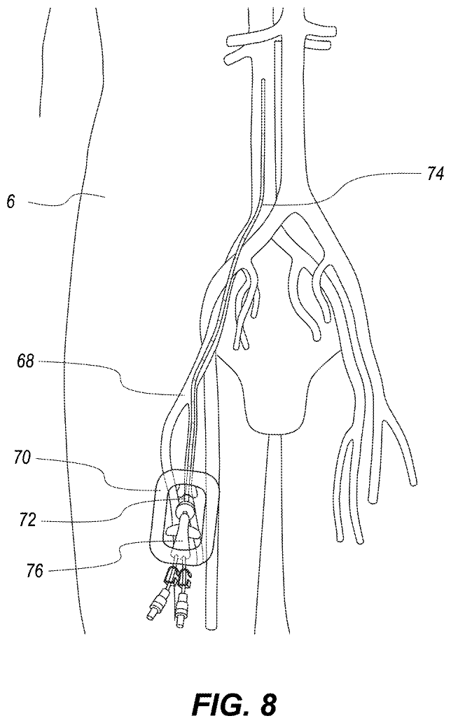

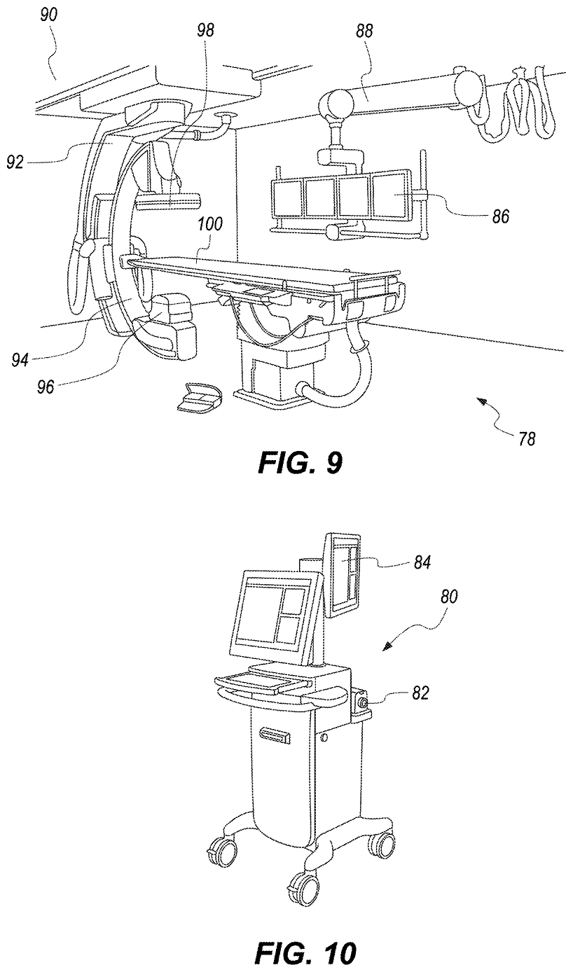

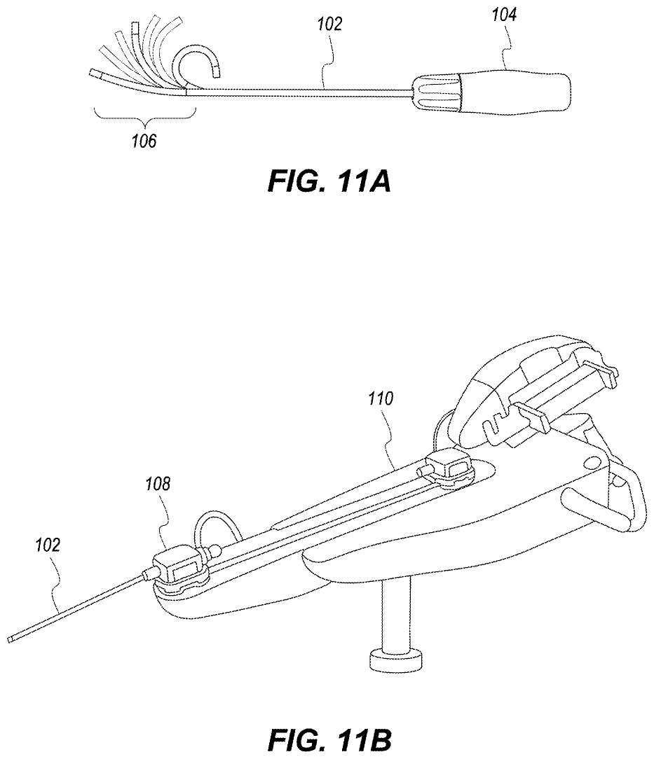

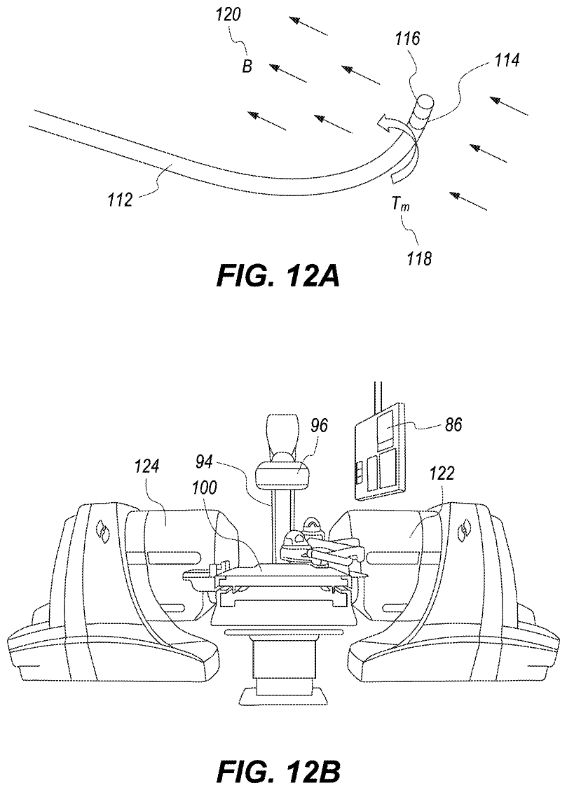

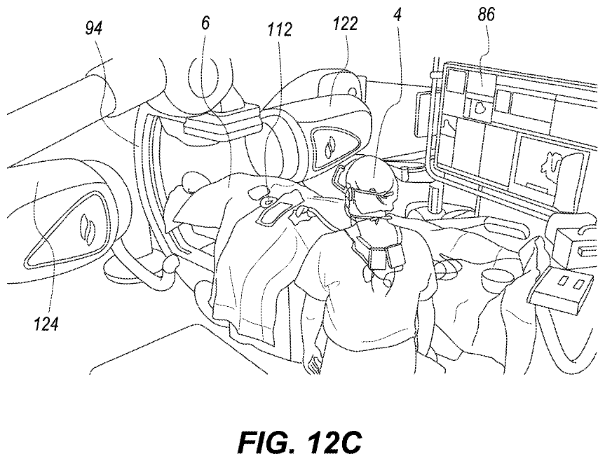

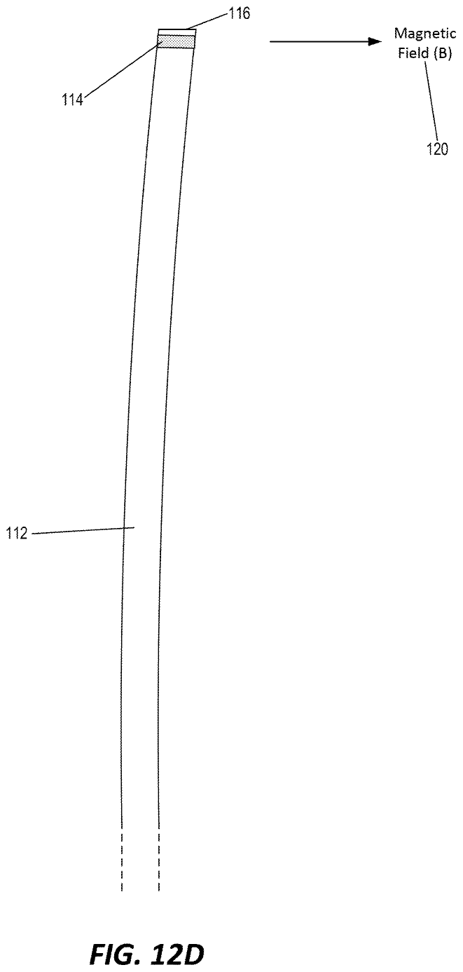

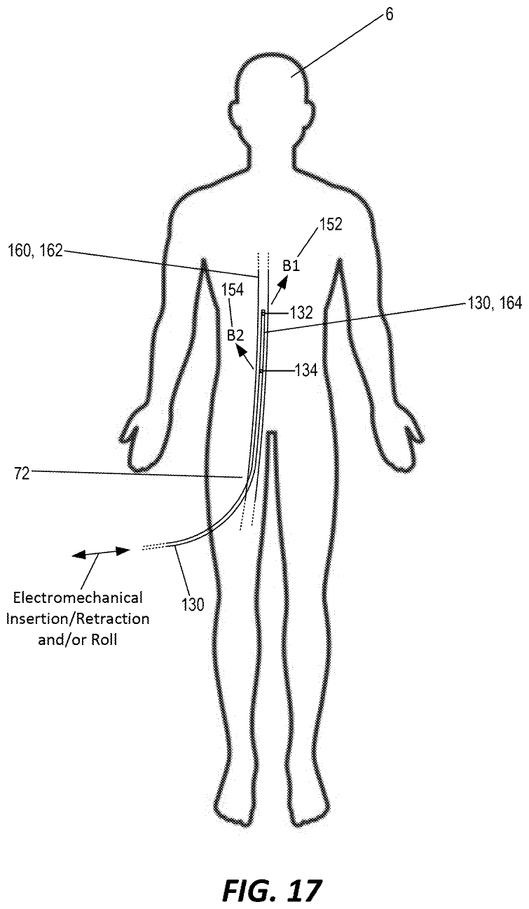

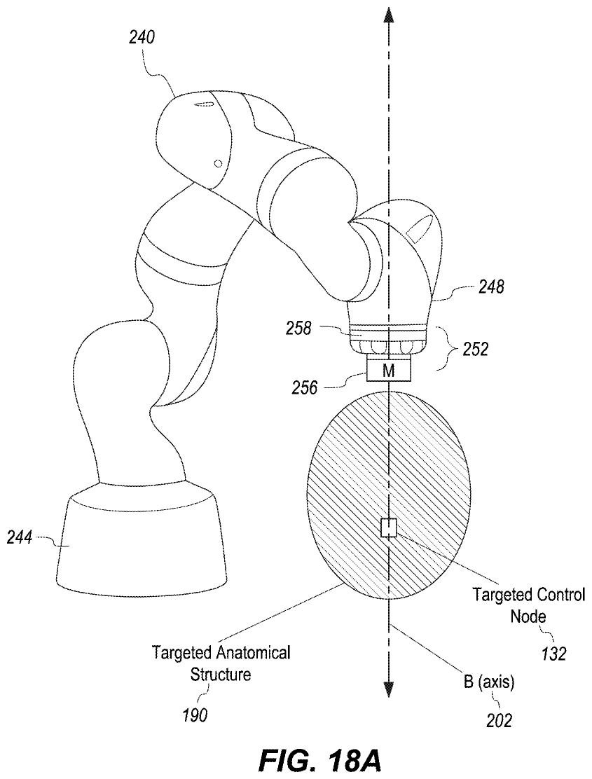

Referring to FIG. 17, as noted above in reference to FIG. 8, for example, various elongate instrument (130) configurations may be utilized to access tissue structures of a patient via endoluminal (160) or endovascular (162) pathways, such as through the arteries of the patient (6). FIG. 17 illustrates an elongate instrument (130) which has been inserted at an access point (72) such as a surgically created so-called “cut-down” access point to an artery such as a femoral artery, so that its distal portions (164) may be navigated up into the patient's (6) cardiovascular system and controllably navigated by use of a plurality of magnetic steerability elements (132, 134), here shown influenced by two vectored and applied magnetic fields B1 and B2 (152, 154, respectively). Insertion and roll degrees of freedom and/or mechanical input to the elongate instrument may be provided from outside of the patient, as shown in FIG. 17.

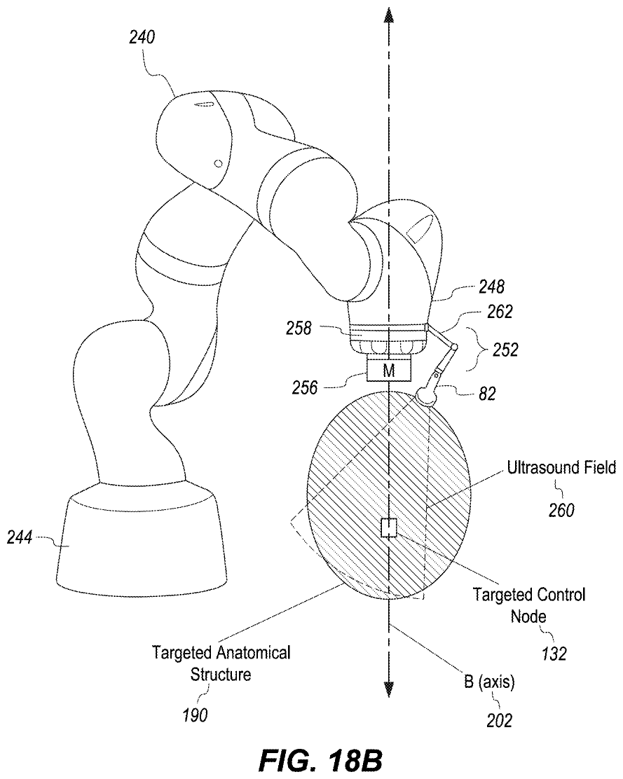

Referring to FIG. 18A, a movable joint assembly or robotic arm (240) similar to that illustrated in FIG. 16 is shown, with a proximal end (244) mountable to an operating table, floor, mobile robot base, ceiling, gantry assembly, or other structure within the operating environment. The distal end (248) may comprise an end effector assembly (252), which may comprise an end effector mounting structure (258) such as a mechanical or electromechanical interface configured for specific removable coupling of tools and/or imaging devices. As shown in FIG. 18A, an end effector magnetic element (256; also denoted as “M”) is coupled to the effector mounting structure (258) and positioned and oriented such that the robotic arm (240) may expose the magnetic field and/or flux axis (202) of this effector mounting structure (258) in a controllable and targeted manner to items such as the depicted targeted anatomical structure (190) of a patient and/or a control node (132) of an elongate instrument which may be positioned inside of such targeted anatomical structure (190).

Referring to FIG. 18B, a configuration similar to that of FIG. 18A is illustrated, with the addition of an ultrasound transducer (82) coupled into position against the targeted anatomical structure (190) utilizing a movable mounting assembly (262; may comprise, for example, a small articulated arm or robotic arm which may be manually or automatically movable relative to its coupling at proximal end to the end effector mounting structure 258; may be at least temporarily fixable into a preferred configuration for usage, and preferably produces image information that may be registered within the same local coordinate system of the overlapping magnetic field emissions; preferably is configured to place the ultrasound transducer 82 into contact against the targeted anatomical structure 190 such that an ultrasound field of view 260 may be utilized to gather images or image-related information pertaining to the targeted anatomical structure 190 and/or the associated targeted control node 132 of the associated elongate instrument). Such a configuration featuring controlled magnetic flux as well as integrated ultrasound imaging using an ultrasound transducer (82) collectively coupled to the robotic arm (240) may be utilized to efficiently image and or locate aspects of an instrument such as a targeted control node (132) during operation and/or navigation, which may be influenced at least in part utilizing the magnetic flux.

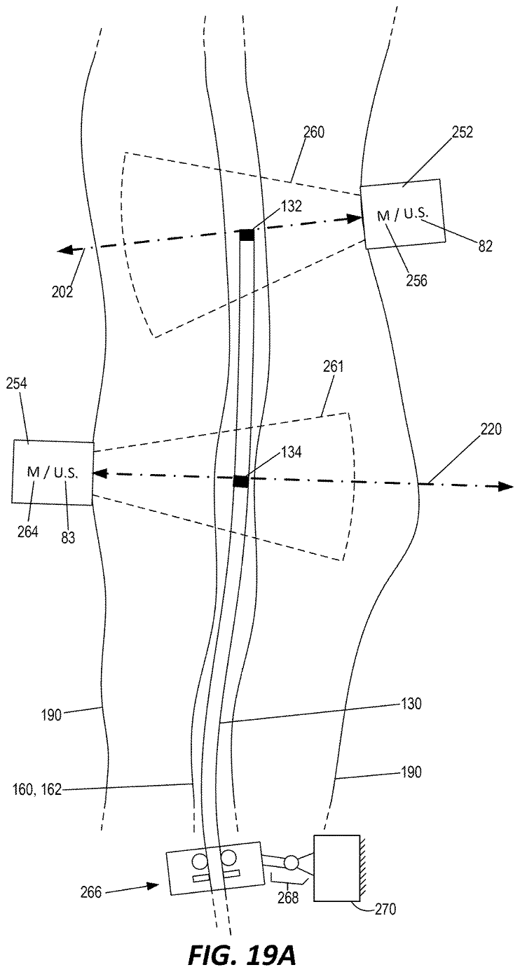

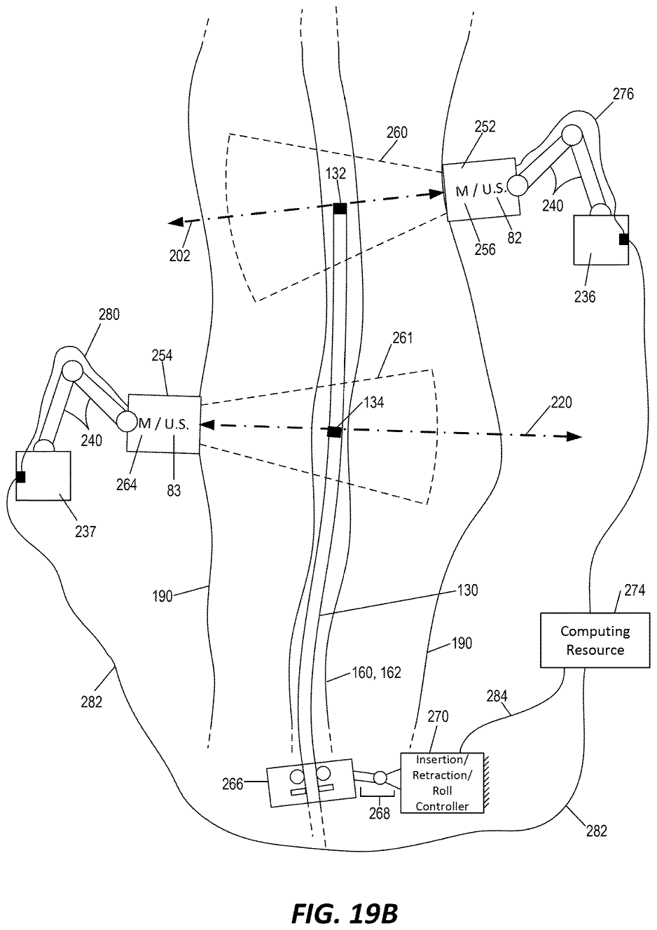

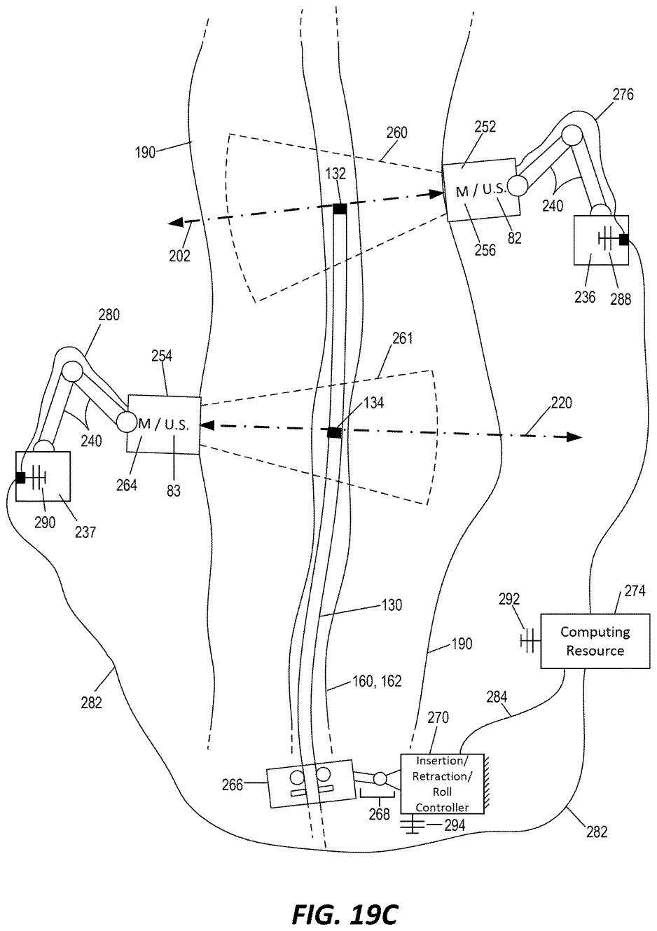

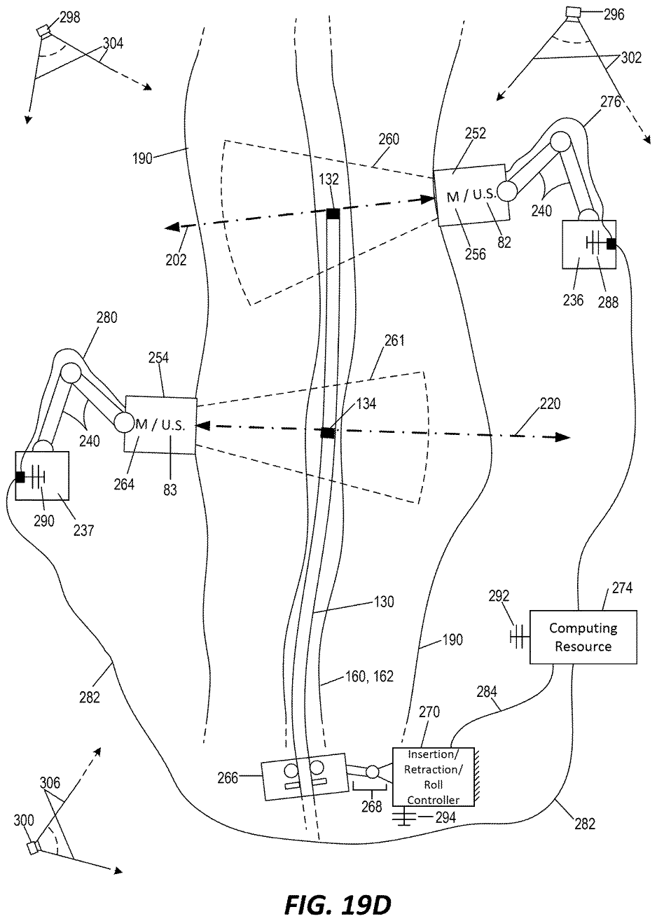

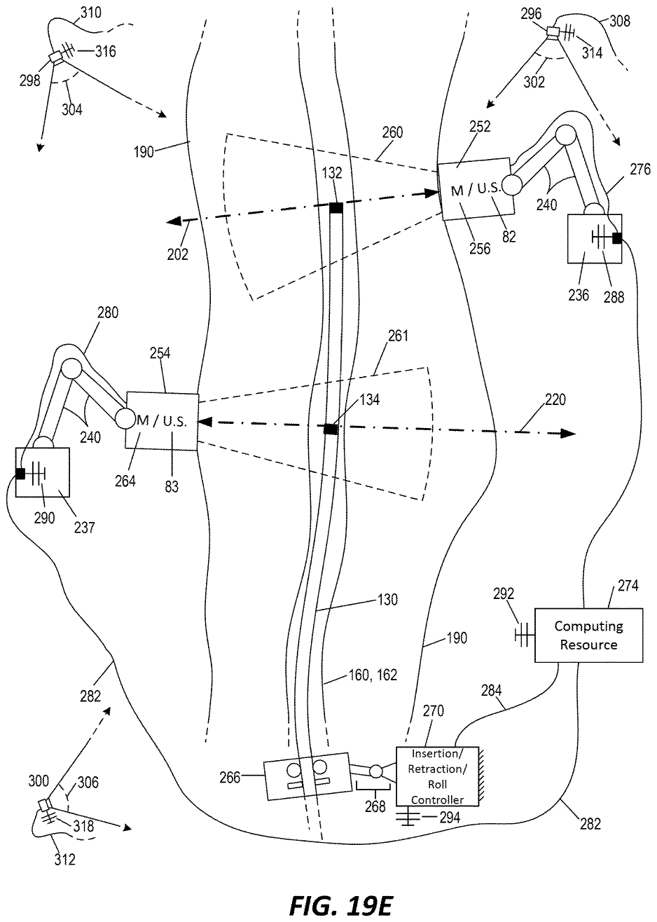

Referring to FIG. 19A, an embodiment is illustrated wherein an elongate instrument (130) is being navigated through a vascular (162) lumen (160) of a patient tissue structure (190) with bi-nodal (i.e., two nodes) steerability provided by two different magnetic flux based steering nodes (256/132; 264/134), as well as insertion and roll degrees of freedom provided by an external driver assembly (266). As shown in FIG. 19A, at one location along the patient tissue structure (190), a first end effector assembly (252), this one featuring an end effector magnetic element (256) configured to create a vectored (202) magnetic flux, here aimed into the patient and toward at least a portion of the elongate instrument (130) to implement magnetic steerability of a first targeted steerability element (132). The depicted first end effector assembly (252) also comprises an ultrasound transducer (82) configured to interface directly with the patient tissue structure (190) and to provide for an ultrasound field of view (260) capturing elements such as the first targeted steerability element (132), and adjacent portions of the elongate instrument (130) and vessel (162) or lumen (160). At another location along the patient tissue structure (190), a second end effector assembly (254), this one featuring an end effector magnetic element (264) configured to create a vectored (220) magnetic flux, here aimed into the patient and toward at least a portion of the elongate instrument (130) to implement magnetic steerability of a second targeted steerability element (134). The depicted first end effector assembly (254) also comprises an ultrasound transducer (83) configured to interface directly with the patient tissue structure (190) and to provide for an ultrasound field of view (261) capturing elements such as the second targeted steerability element (134), and adjacent portions of the elongate instrument (130) and vessel (162) or lumen (160).

Also shown in FIG. 19A is an instrument insert/roll control interface (266) which may be coupled (such as via a coupling assembly 268 which may comprise one or more movable joints) to a support member or structure (270), which may be coupled to a structure such as a floor of an operating room, or a stable mounting tower or gantry (either of which may be mounted to the ceiling and/or floor, for example) to provide relative stability to the insert/roll control assembly (266) and intercoupled proximal portion of the elongate instrument (130) passing through the insert/retract/roll control assembly (266) as shown. The support member or structure (270) may house or be operatively coupled to an insert/retract/roll controller subsystem as shown in FIG. 19B.

Referring to FIG. 19B, a configuration similar to that of FIG. 19A is illustrated, with the addition of automatically controllable movement control assemblies such as robotic arms (240, 242) coupled to robotic arm bases (236, 237, respectively, as shown) and configured to electromechanically position and orient the end effector assemblies (252, 254, respectively, as shown) relative to the tissue structure (190), lumen (160), vessel (162), and/or steerability element (132, 134, respectively, as shown) structures to assist with navigation and control of the elongate instrument (130) relative to the tissue structures of the patient (190, 160/162). The end effector assemblies (252, 254) and robotic arms (240, 242, respectively) may be operatively coupled (such as via wired connectivity elements 276, 278, 280, 282) to a computing system or computing resource (274) along with (wired connectivity element 284) the insert/retract/roll interface (266) assembly controller (272), here shown optionally housed within or coupled to the support member (270).

Referring to FIG. 19C, a configuration similar to that of FIG. 19B is illustrated, with the addition of wireless connectivity transceivers (288, 292, 294, 290) to illustrate that various elements may be operatively coupled using wireless connectivity (such as configurations based upon IEEE 802.11 standards, Bluetooth™ or other radiofrequency nearfield standards, for example) in addition to or alternatively to the wired connectivity elements (276, 278, 280, 282, 284).

Referring to FIG. 19D, a configuration similar to that of FIG. 19C is illustrated, with the addition of a plurality of image capture devices (296, 298, 300) positioned and having fields of capture or view (302, 304, 306) oriented to capture image information pertaining to the various system components (such as robotic arm/end effector assemblies 236/240/252, 237/242/254; portions of the targeted tissue structure 190; portions of the instrument insert/retract/roll interface (266), support structure (270), controller (272), and/or computing resource (274). The image information from these image capture devices (296, 298, 300) may be utilized for various aspects of system control. For example, one more of the image capture devices (296, 298, 300) may comprise a depth or stereo capture device configured to provide Z-axis depth information (such as those available from Intel Corporation under the tradename RealSense™) in addition to conventional X-Y axes pixel position/intensity/coloration information relative to the position and orientation of the device (296, 298, 300). Referring to FIG. 19E, these image capture devices (296, 298, 300) may be operatively coupled to other elements such as the computing resource (274), such as via wired connections (308, 310, 312), and/or via wireless connectivity (such as configurations based upon IEEE 802.11 standards, Bluetooth™ or other radiofrequency nearfield standards, for example) using RF transceiver elements as shown (314, 316, 318, respectively).

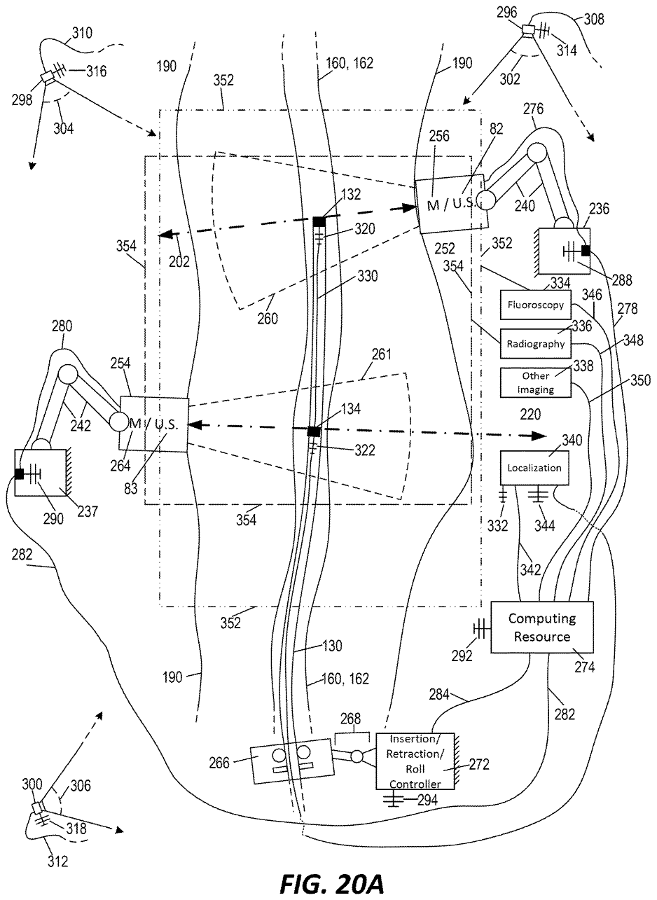

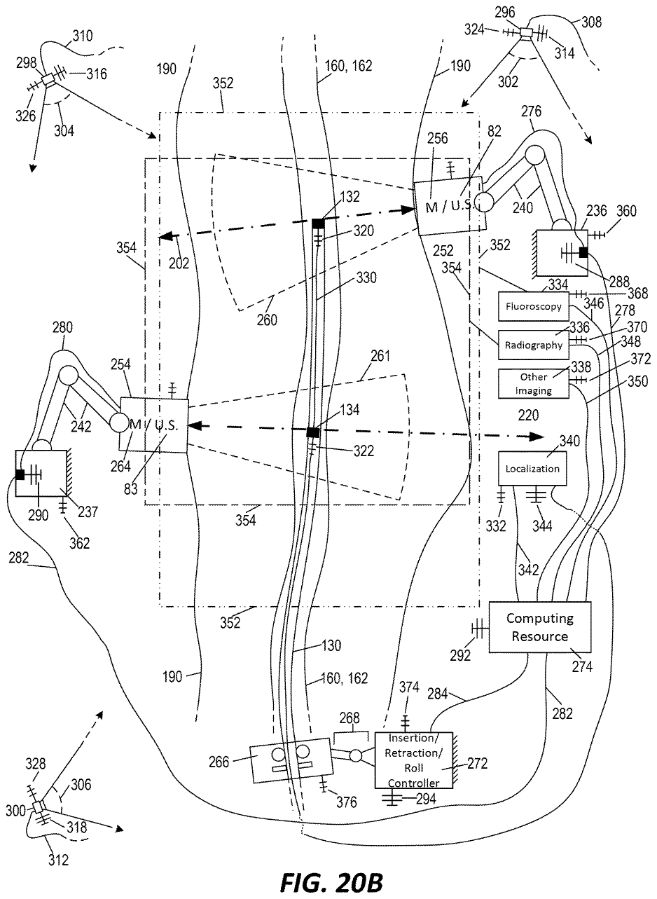

Referring to FIG. 20A, a configuration similar to that of FIG. 19E is illustrated, with the addition of exemplary integrated imaging (fluoroscopy 334, radiography 336, and other 338, such as computed tomography, magnetic resonance, hall-effect sensing, and/or additional ultrasound) and localization (340) systems which are operatively coupled (such as by wired connectivity (346, 348, 350, 342), or wireless connectivity (for example, transceivers as shown to wirelessly connect the localization system 344, computing resource 292, and insert/retract/roll controller 294, and robotic arm systems 288/290 for automatically positioning and/or orienting the end effector assemblies 252/254 for use in navigation and imaging of the elongate instrument 130). The computing system (274) may be configured to receive preoperative and/or intraoperative image information from connected imaging systems, to automatically register (or alternatively allow for operator-assisted registration, such as via manually-enhanced registration of portions of image information relative to each other given certain aspects of this image information which may be visualized by an operator using a visual user interface) this imaging information relative to a coordinate system (for example, a global coordinate system designated to be fixed within the operating room, such as fixed relative to the floor and/or ceiling; or fixed relative to a fixable structure such as an operating table) so that it may be collectively utilized to assist operators in visualizing the subject patient anatomy and associated structures (for example, plaques, implants such as stents, other instruments such as needles, catheters, infusion devices, etc) relative to operative instrumentation such as portions of an elongate instrument (130), as well as used for navigating portions of the elongate instrument (130), such as via manual or automatic navigation inputs to, for example, modulate magnetic field application and/or vectoring and/or insertion/retraction/roll of the elongate instrument using the proximally-located driver interface (266). The depicted localization system (340) may comprise a radiofrequency-based localization system, such as those available from providers such as the BioSense division of Johnson & Johnson, or the Ascension division of Northern Digital, Inc., which is configured to broadcast electromagnetic (“EM”) radiation (transceiver shown as element 332 in FIG. 20A) from one or more transmitters, and to receive portions of such broadcasted radiation using so-called “EM-sensors” (shown in FIG. 20A as elements 320 and 322 for tracking two portions of the elongate instrument; each of which may comprise, for example, a composition of coils configured to be oriented at orthogonal or other orientations relative to each other within each sensor, so that detected currents associated with the composition of coils may be utilized to estimate position and orientation in three-dimensional space, such as relative to a selected global coordinate system). Electromagnetic transmissions of such a tracking system (340) may be time-multiplexed with other applied electromagnetic radiation or flux (such as from the depicted steering control elements 256, 264) to avoid interference, and/or the system may be configured to accommodate simultaneous superposition of such electromagnetic radiation or flux.

Referring to FIG. 20B, a configuration similar to that of FIG. 20A is illustrated with the addition of further localization integration and tracking capability for additional closed-loop sensing and monitoring of position and/or orientation of elements, showing EM-sensors located also on various additional system elements, such as image capture devices 296, 298, 300 (associated EM-sensors 324, 326, 328, respectively), end effector assemblies 252, 254 (associated EM-sensors 364, 262, respectively), robotic bases 236, 237 (associated EM-sensors 360, 362, respectively), robotic bases 236, 237 (associated EM-sensors 360, 362, respectively), instrument insert/retract/roll actuation interface 266 (associated EM-sensor 376), support base 270 and/or insert/retract/roll controller 272 (associated EM-sensor 374), as well as the imaging systems and/or components thereof, such as fluoroscopy 334 (associated EM-sensor 368), radiography 336 (associated EM-sensor 370), and/or other imaging 338 (associated EM-sensor 372).

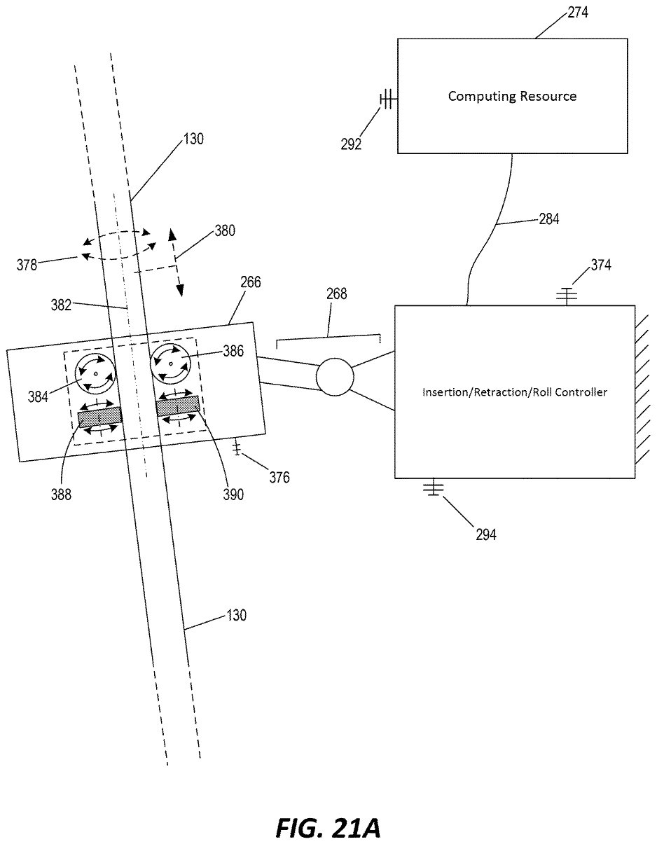

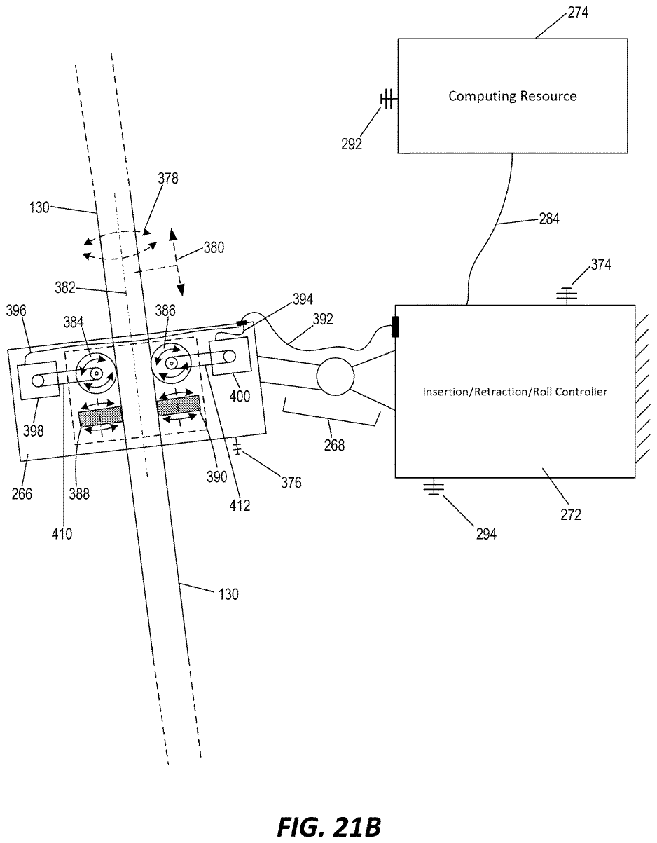

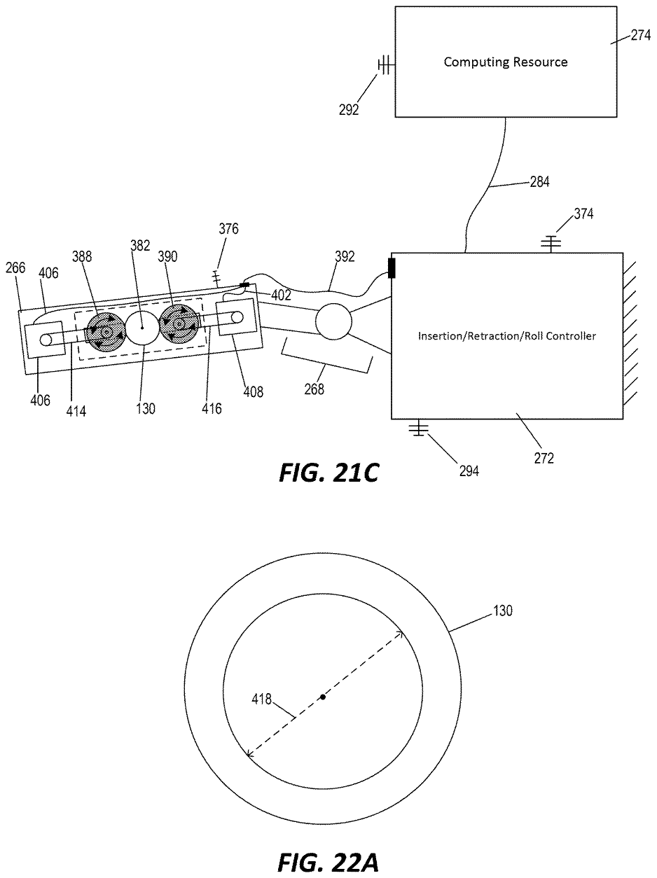

Referring to FIGS. 21A-21C, further aspects of an insert/retract/roll actuation interface (266) and associated controller (272) are illustrated. As shown in FIG. 21A, a portion of an elongate instrument (130) may comprise a generally cylindrical outer shape, and may be placed through a portion of the insert/retract/roll actuation interface assembly (266) so that roll (378; in either direction, clockwise or counterclockwise, about a central axis 382 of the instrument 130) of this instrument (130), as well as insert or retract (380), may be controllably and electromechanically actuated. As shown in FIGS. 21A and 21B, insert or retract (380) may be imparted to the instrument (130) using a plurality of interfacing wheel members (384, 386) which may be operatively coupled (such as via drive belts 410, 412) to motors (398, 400) which are operatively coupled (such as via wired connectivity 394, 396, 392, 284) to the insertion/retraction/roll controller (272) and computing resource (274). Referring to FIG. 21C, an orthogonal view of the insert/retract/roll actuation interface assembly (266) illustrates that roll actuation may be imparted using another plurality of physically interfaced and movably coupled wheel members (388, 390) which may be operatively coupled (such as via drive belts 414, 416) to motors (406, 408) which are operatively coupled (such as via wired connectivity 402, 406, 392, 284) to the insertion/retraction/roll controller (272) and computing resource (274).

With a configuration such as that illustrated in FIGS. 21A-21C and 20B, for example, a typical procedure may entail gathering preoperative imaging information such as via magnetic resonance imaging (“MRI”), computed tomography (“CT”), ultrasound, and/or planar radiography. The image information pertaining to these modalities may be co-registered to the anatomy of the patient by using anatomical markers, such as well known anatomic features which may be at least partially captured during imaging. Image information may be registered relative to other image information utilizing fiducials (such as as implanted or nearby radio-opaque pins or markers) and/or known imaging system component positions and/or orientations within a global coordinate system (which may be tracked, for example, using a localization tracking system 340/332 and sensors such as 368, 370, 372, 374, 376, 360, 362, 324, 326, 328, 320, 322), such as one defined to be that of the operating room floor, ceiling, or other fixed structure. Co-registered image information pertaining to the patient anatomy may be utilized for multi-modal preoperatively planning and visualization, as well as subsequent intraoperative visualization during procedure execution. Image information from multiple modalities may be co-registered, and discrete and/or overlapping image information from a common modality may be co-registered (in other words, a plurality of discrete so-called “slices” or portions of image information, such as from ultrasound, CT, MRI, or radiography, from discrete anatomical locations may be utilized and visualized together as they are visualized relative to each other within the same coordinate system; they may be visually spaced apart, visually fused in the case of geometric overlap, stitched together relative to each other, and/or interpolated so that a unified view is made available to operators for visualization, planning, and instrument operation and/or navigation). For example, at intervention time, co-registered image information from one or more modalities may be examined and co-registered with coordinate systems for position, orientation, and movement visualization information pertaining to interventional tools such as cutting tools, guidewires, and elongate instruments (130). In other words, the interventional tools may be visualized within and “driven”, navigated, and/or utilized within the same global coordinate system as the co-registered image information for intuitive monitoring and operation by operators as they view the anatomy and tools within a display or other visualization system.

Referring again to a system such as that disclosed in FIG. 20B, with preoperative and intraoperative image information co-registered, and the elongate instrument (130) co-registered and operating in real or near-real time relative to tracking (320, 322, 376, 374, 332, 340), the elongate instrument (130) may be navigated (such as via magnetic-flux based steerability 256/202, 264/220 as well as electromechanical insert/retract/roll 266, 272); all coordinated by the intercoupled computing resource (274). The computing resource (274) may be configured to dynamically account for magnetic flux or field interference from the applied and vectored steerability sources (256, 264) at the localization system (340/332; 320, 322, 376, 374) using superimposition (i.e., known interference may be backed out in the determination algorithm) and knowledge of the applied/vectored flux as well as other field interference elements such as ferromagnetic materials within various elements (such as steerability elements 132, 134); time-based multiplexing may also be utilized to shunt interfering magnetic flux signals during an electromagnetic localization read sequence as well, for example.

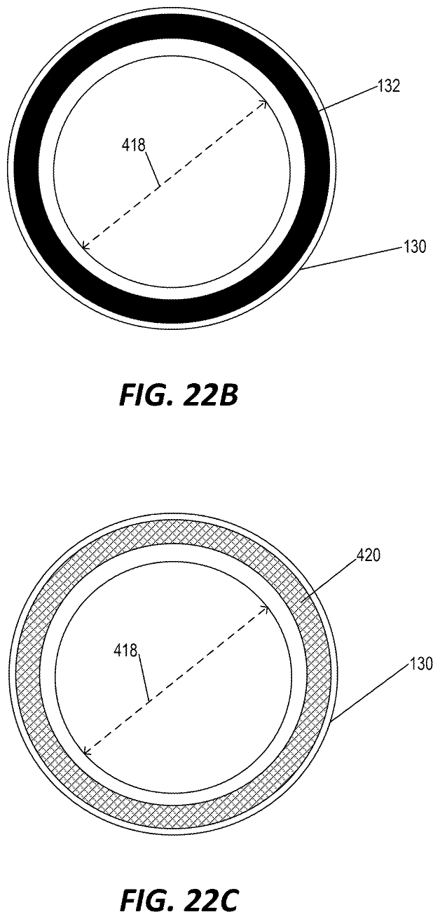

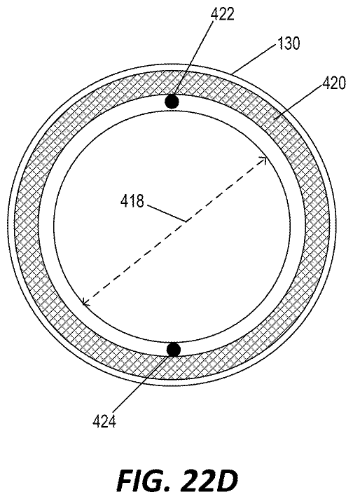

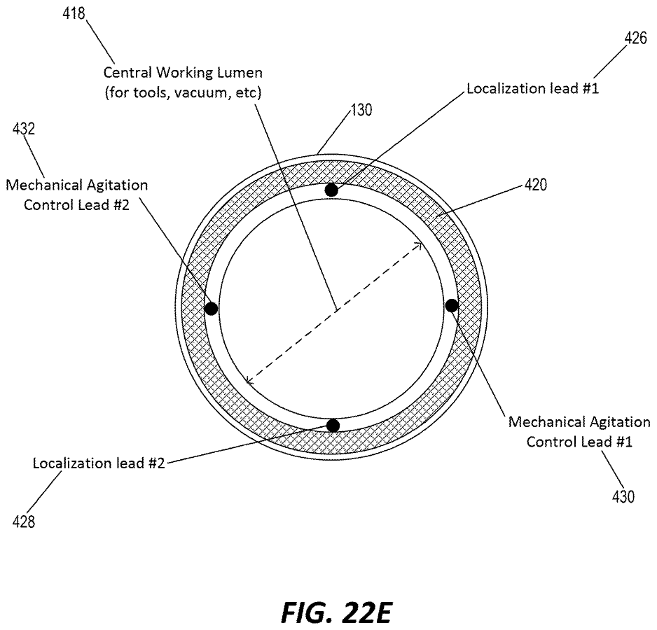

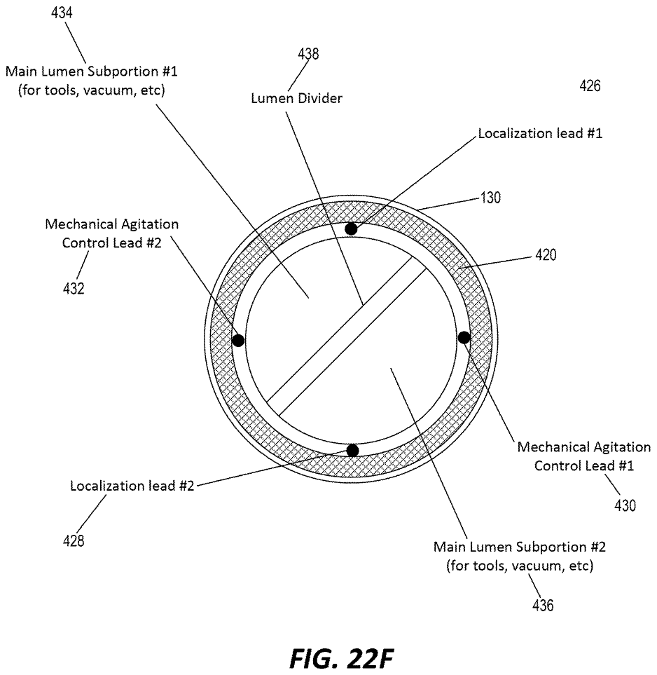

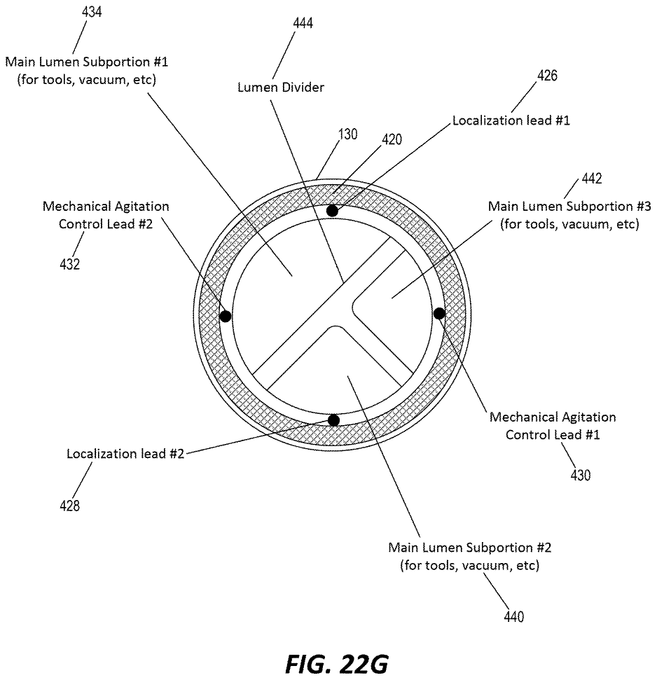

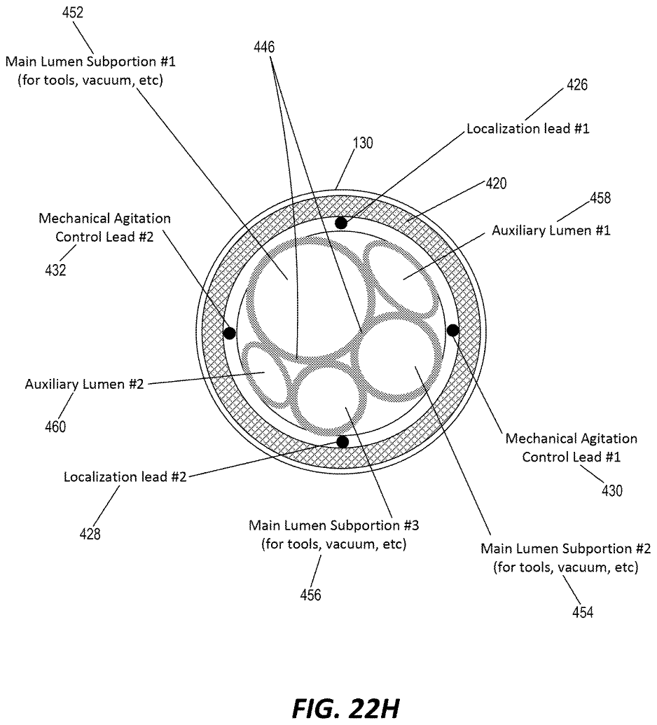

Referring to FIG. 22A, an illustrative cross-sectional view of a portion of a suitable elongate instrument (130) is shown defining a primary working lumen having an inner diameter (418) through which various tools or other instruments may be placed and/or operated, or through which vacuum or perfusion of fluids may be performed. The elongate instrument (130) may comprise one or more layers of polymeric material, such as polyethylene, which may be formed using extrusion, co-extrusion, injection molding, or other techniques. Referring to FIG. 22B, a configuration similar to that of FIG. 22A is illustrated, with the addition of a ring-like ferromagnetic element (132) embedded within the given portion of the elongate instrument (130) wall. Referring to FIG. 22C, a configuration similar to that of FIG. 22A is illustrated, with the addition of a ring-like section of structural material (420) embedded within the given portion of the elongate instrument (130) wall to provide a different modulus (i.e., by virtue of the depicted composite configuration with two or more different materials comprising the wall of the elongate instrument 130) to the overall mechanical performance of this portion. Sections or portions of embedded structural material (420) such as this may be included to improve overall kink resistance or resistance to compression under loading, for example.

Referring to FIG. 22D, a configuration similar to that of FIG. 22C is illustrated, with addition of a plurality of connectivity leads (422, 424), such as copper or other conductive leads which may be utilized to conduct signals to and from elements such as the localization sensors (320, 322) illustrated in FIG. 20B, for example; these leads may comprise single conductive elements, or may comprise a plurality of elements, such as bundles of multiple discrete signal conductors, which may be patterned, for example, in twisted or braided elongate configurations. These leads (422, 424) may be positioned cross sectionally at approximately diametrically opposed positions as shown to reduce asymmetries in terms of the bending or other physical performance of the overall elongate instrument (130). Referring to FIG. 22E, a configuration somewhat similar to that of FIG. 22D is illustrated, wherein two connectivity or localization leads (426, 428) are positioned approximately diametrically opposed in cross section and configured to provide for communication with localization sensors; also positioned approximately diametrically opposed in cross section are two mechanical agitation or control leads (430, 432) which may be utilized, for example, as pull-wires, push-rods, or the like, to transfer loads along selected lengths of the elongate instrument (130) which may be utilized for various functions such as steerability or bending resistance.

Referring to FIG. 22F, a configuration similar to that of FIG. 22E is illustrated with the addition of a lumen divider (438) configured to define two discrete main lumen subportions (434, 436), to accommodate separate tools, infusion, vacuum, or the like; in other words, depending upon the longitudinal configuration (FIG. 22F being a particular cross sectional representation along a given longitudinal position) two separate lumens may be defined using the divider (428), which may be constructed using polymeric co-extrusion, for example.

Referring to FIG. 22G, a configuration is illustrated wherein a lumen divider (444; also may be formed using polymeric co-extrusion techniques, for example) defines three discrete lumen subportions (434, 440, 442) to provide for further discrete operational space along the elongate instrument (130).

Referring to FIG. 22H, a configuration is illustrated wherein a lumen divider complex (446; also may be formed using polymeric co-extrusion techniques, for example) defines five discrete lumen subportions (452, 454, 456, 458, 460) to provide for additional discrete operational space along the elongate instrument (130).

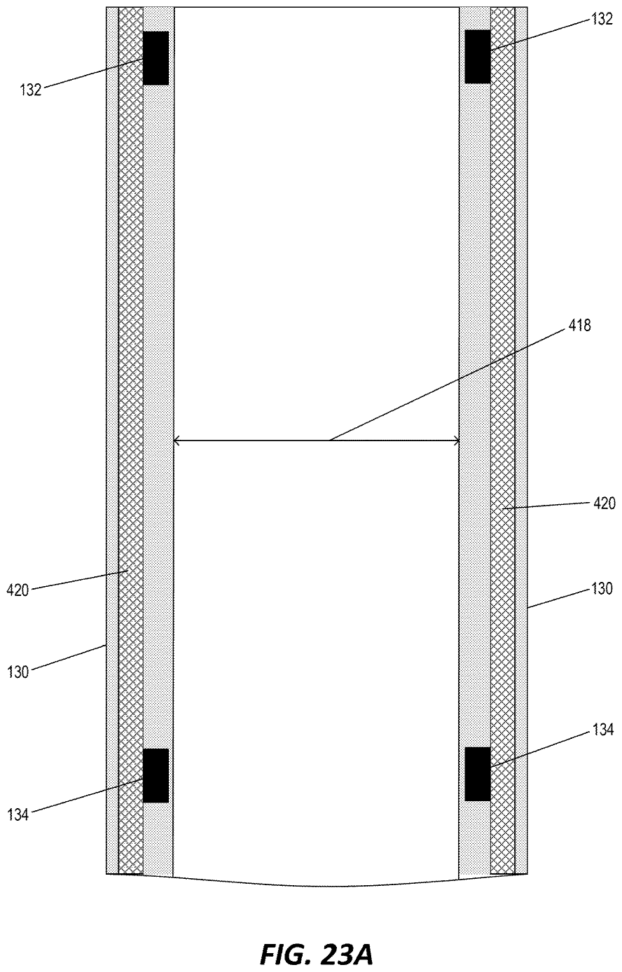

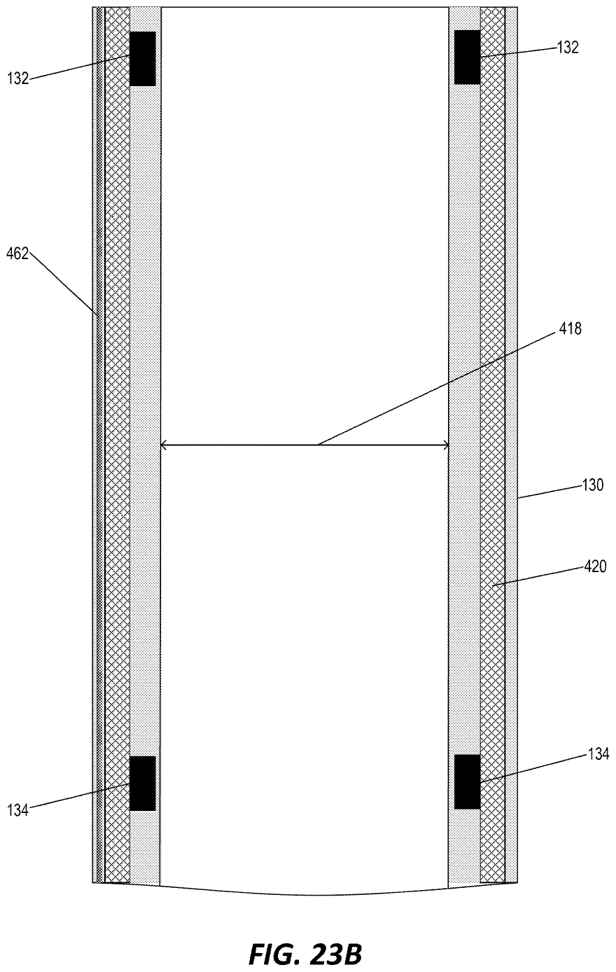

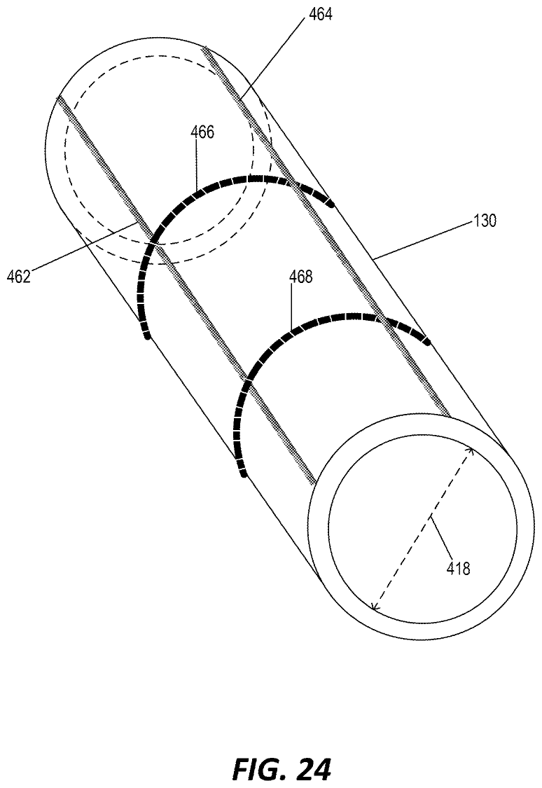

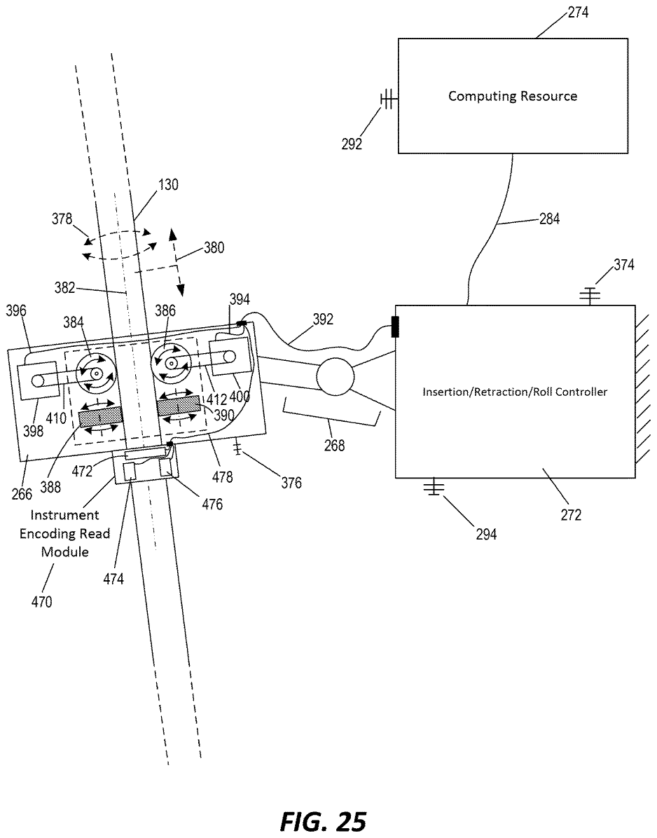

Referring to FIG. 23A, a longitudinal portion of an elongate instrument (130) is illustrated in cross section featuring two ring-like ferromagnetic elements (132, 134) along with a structural layer (420) embedded within the wall of the elongate instrument (130), the wall defining a working lumen with an inner diameter (418) as shown. Referring to FIG. 23B, to assist with determination of insertion/retraction positioning, an encoded member (462) may comprise a portion of the elongate instrument (130) wall and may comprise encoded information which may be read by one or more of the reader instruments (472, 474, 476) comprising a read module (470) such as is illustrated in FIG. 25. Referring to FIG. 24, an elongate instrument (130) may comprise a plurality of longitudinal encoded members (462, 464) for determining insert/retract positioning, as well as one or more circumferential encoded members (466, 468) for determining roll positioning. The configuration illustrated in FIG. 25 comprises features similar to that of FIG. 21B, with the addition of an instrument encoding read module (470) comprising a plurality of reader elements (472, 474, 476) positioned and oriented to assist with determination of insert/retract and roll positioning in real or near-real time. For example, the configuration of FIG. 25 features two or more reader elements (474, 476) configured to read elongate encoded members for insert/retract, as well as at least one reader element (472) configured to read circumferential encoded members for roll determination.

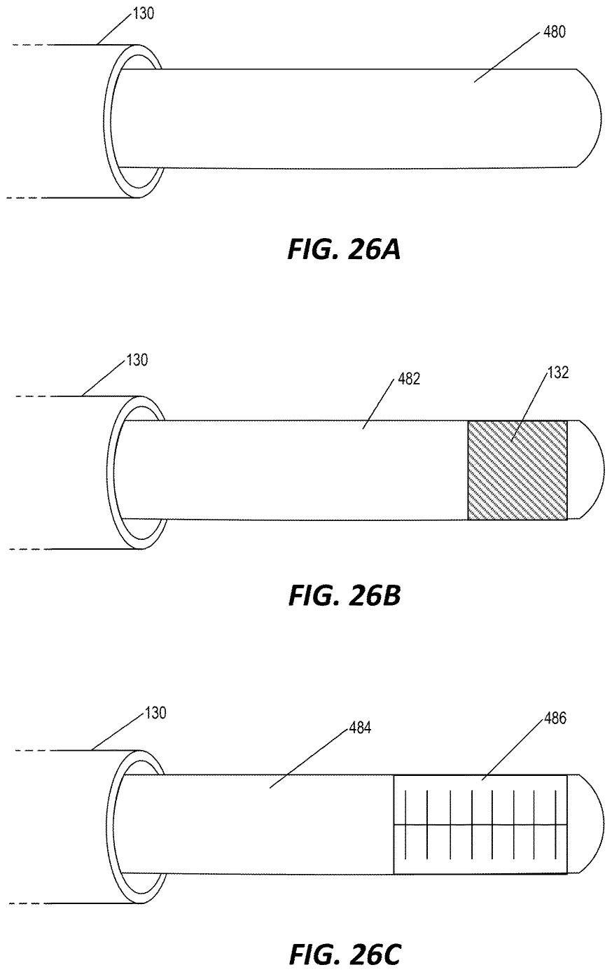

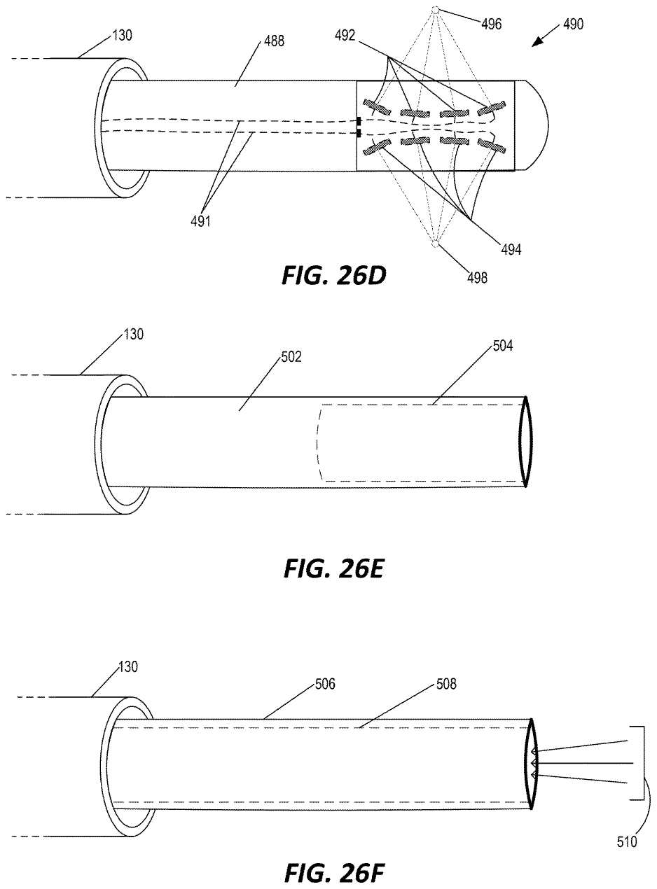

Referring to FIGS. 26A-29E, various different tools or instruments may be utilized through a working lumen of a subject elongate instrument (130) configuration to address various procedural challenges. For example, referring to FIG. 26A, an elongate probe style tool (480), such as a metallic wire or polymeric member, here depicted having an atraumatic/rounded distal portion, may be utilized as a working tool through an elongate instrument (130) to probe, press, and otherwise physically investigate and/or intervene with a tissue structure or other structure (such as an implant, plaque deposit, or other instrument). FIG. 26B illustrates a configuration wherein a ferromagnetic probe tool (482) is placed through a working lumen of an elongate instrument (130). The ferromagnetic probe tool (482) may comprise one or more ferromagnetic elements (as shown in FIG. 26B, element 132) which may be configured to interact with applied and vectored steerability magnetic fields or magnetic flux, for example, as such tool (482) is utilized in intraoperative functions such as to probe, press, and otherwise physically investigate and/or intervene with a tissue structure or other structure (such as an implant, plaque deposit, or other instrument).

Referring to FIG. 26C, a resonant tool (484) configuration is illustrated positioned through a working lumen of an elongate instrument (130). The resonant tool (484) comprises one or more antennae or resonant members (486) configured to cause controlled and predicted oscillation, or oscillatory motion, of the resonant tool (484) and other items which may be in contact therewith (such as a tissue structure or other structure, such as an implant, plaque deposit, or other instrument) when stimulated with appropriate and prescribed (i.e., prescribed to match the antennae or resonant members to induce expected oscillatory motion) stimulating radiation or signalling.

Referring to FIG. 26D, a local focused emission probe (490) configuration is illustrated positioned through a elongate instrument (130). The local focused emission probe (490) may comprise a plurality of radiation emission elements (492, 494) which may be positioned and oriented to focus emitted radiation upon one or more nearby focal points (496, 498, respectively). The emission elements (492, 494) may be configured to emit at various selected and/or prescribed wavelengths to assist with radiation-based intervention of targeted tissues or other structures (such as plaques) which may be positioned at or adjacent to the focal points (496, 498). The emission elements (492, 494) may comprise materials such as piezoelectric materials selected to produce relatively high-frequency vibratory wavefronts at wavelengths known as “ultrasonic” (i.e., ultrasound) such that a high-intensity focused ultrasound intervention may be conducted at the focal points (496, 498). The emission elements (492, 494) may be coupled to a controller and/or stimulation power source via use of conductive leads (491) as shown.

Referring to FIG. 26E, a cannulation probe (502) is illustrated positioned through a working lumen of an elongate instrument (130). The cannulation probe (502) may comprise an open interior volume (504) and distal portion configured to be able to probe and potentially capture items or portions thereof that are encountered. In various embodiments, the distal portion may be atraumatic and rounded in shape profile; in other embodiments it may be substantially sharp such that it may be utilized as a somewhat sharpened instrument to carve or scrape against another object, such as a tissue structure or plaque.

Referring to FIG. 26F, a vacuum probe (506) configuration is illustrated positioned through a working lumen of an elongate instrument (130), the vacuum probe (506) defining a vacuum lumen (508) therethrough, such that vacuum flow (510) may be utilized to pull, remove, or urge nearby structures or fluids into the vacuum lumen (508) and potentially transfer them through the entire length of the vacuum probe (506) to a proximal location, such as a vacuum result holding reservoir. For example, in various embodiments, a vacuum probe (506) may be utilized to remove portions or all of an embolic, thrombus, or clot structure from a location within a vessel or other tissue structure, with confirmation that certain material has been removed available through use of a vacuum result holding reservoir which may be located proximally and extracorporeally.

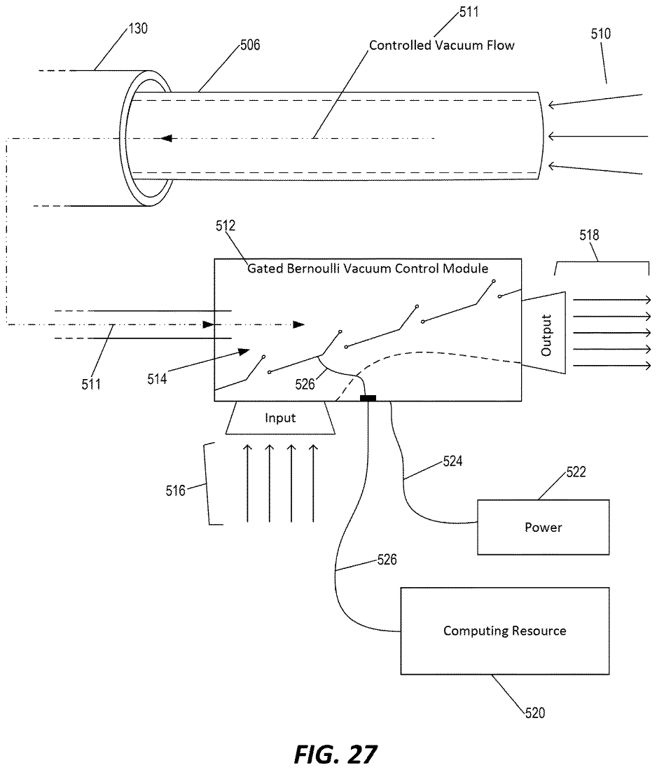

Referring to FIG. 27, a vacuum probe (506) embodiment is illustrated with controlled vacuum flow (511) controllably modulated via a control module such as a gated Bernoulli vacuum control module (512), as illustrated, which may, for example, comprise one or more computer controlled (526, 520) electromechanical (524, 522) valves (514) configured to utilize the Bernoulli relationships between pressure and velocity to create highly controlled vacuum loading and flow (511). In other words, input flow (516) may be pressurized into an input flow channel with constant or variable input velocity and then forced into a compression channel and past the one or more valves (514) creating variable vacuum flow (511) depending upon the valve conditions (i.e., open, partially open, shut, etc; for example, with all valves 514 shut, the vacuum flow 511 should be close to zero; with all valves 514 fully open, the vacuum flow 511 should be at a maximum) before being forced out of an output or exit portal (518). The valve or plurality of valves (514) may comprise high-frequency controllable reed valves or gates to provide the potential for significantly gated flow and pulsatory flow patterns which may be useful in vacuum-manipulation of items such as embolisms/clots/thrombus, which may have certain viscoelastic properties, as well as fluidic and adhesion properties.

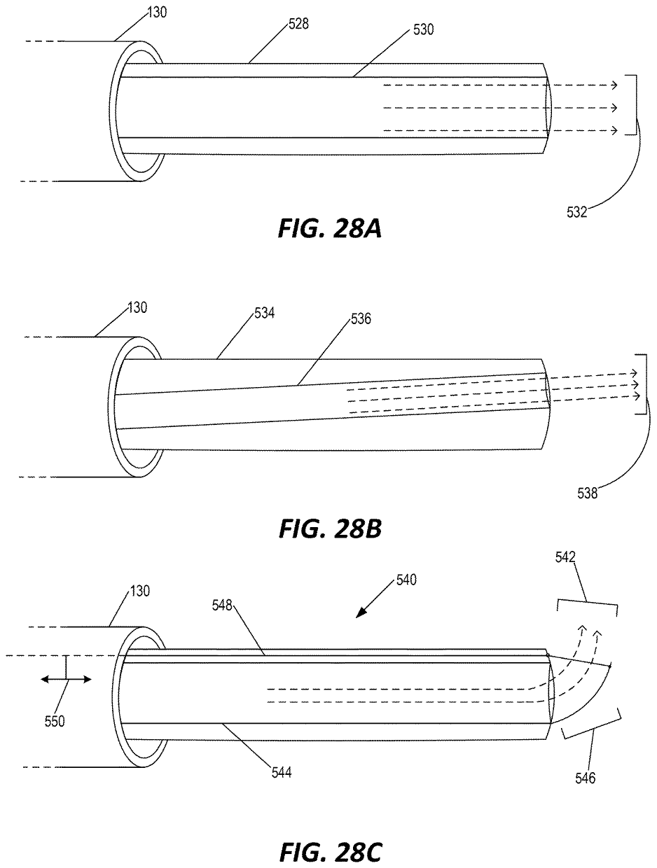

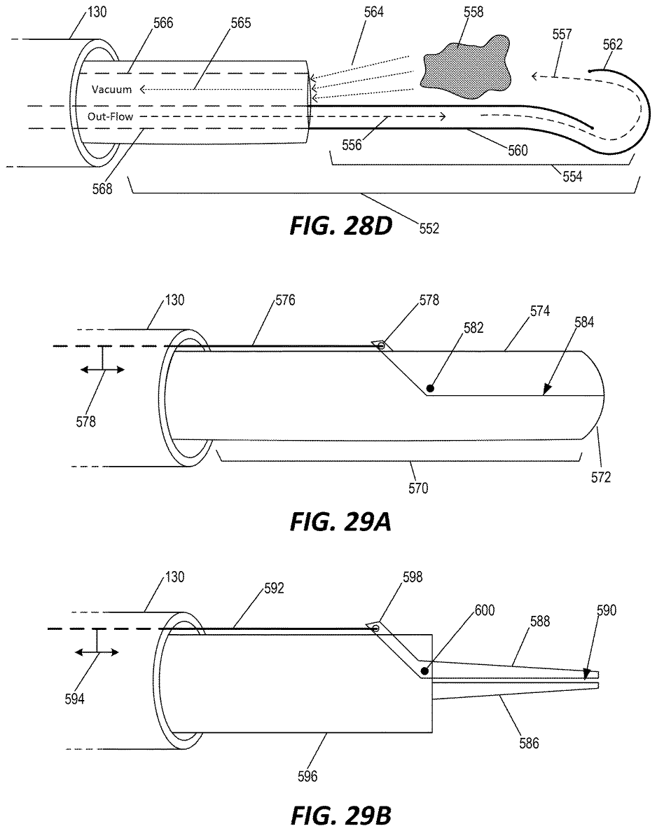

Referring to FIGS. 28A-28D, various fluid perfusion probe configurations are illustrated which may be useful in diagnostic as well as interventional operational steps or phases as positioned through a working lumen of an elongate instrument (130). Referring to FIG. 28A, in one embodiment, a perfusion probe (528) may define a perfusion lumen therethrough (530) which may be configured to focus a jet or flow pattern (532) of controllably expelled fluid such as saline for use in various diagnostic and/or interventional steps, such as perfusing with a medication, contrast agent, or other material, or utilizing a fluid or flowable compound to assist in freeing and/or manipulating a tissue structure or other structure, such as a embolism, clot, thrombus, or portion thereof.

Referring to FIG. 28B, another perfusion probe (534) configuration is illustrated having a smaller and more focused perfusion lumen (536), here positioned at an orientation such that the focused jet or flow pattern (538) of expelled fluid or compound may reach targets more broadly than straight out the distal portion of the probe (534; i.e., with roll of the associated elongate instrument 130, and/or roll of the probe 534 itself, the flow pattern 538 may be oriented toward a variety of targets within reach of the distal end of the probe 534). Referring to FIG. 28C, another perfusion probe (540) embodiment is illustrated having an adjustable flow pattern (542) vector configured to facilitate aiming of the flow pattern (542) vector through use of a re-orientation control member (548; may be a tension member such as a pullwire which may be extended proximally through the elongate instrument 130 that it may be pulled and/or manipulated 550 by an operator manually or electromechanically, such as via use of a controllable motor and capstan configuration) that is coupled to a flexible/re-vectorable distal portion (546) which essentially forms an aimable flume for the exiting flow (542) coming through the perfusion lumen (544).

FIG. 28D illustrates another configuration wherein controlled outflow may be utilized along with vacuum to comprise a capture and/or removal assembly, or infusion/vacuum probe assembly (552) which may be delivered and stabilized using an elongate instrument (130). As shown in FIG. 28D, parallel outflow or perfusion (556) and inflow or vacuum (566) lumens may be positioned through the probe (552), with perfusion or out-flow advanced forward through tubular portion (560) which extends to a flow redirection portion (562) causing a perfusion fluid pattern (557) which is oriented in a substantially reversed direction toward the vacuum inlet pattern (564), such that an object such as a clot (558) may be perfused, dislodged, disrupted, or otherwise urged toward the vacuum inlet pattern (564) and preferably into the vacuum lumen (566) for removal and/or holding, for example.

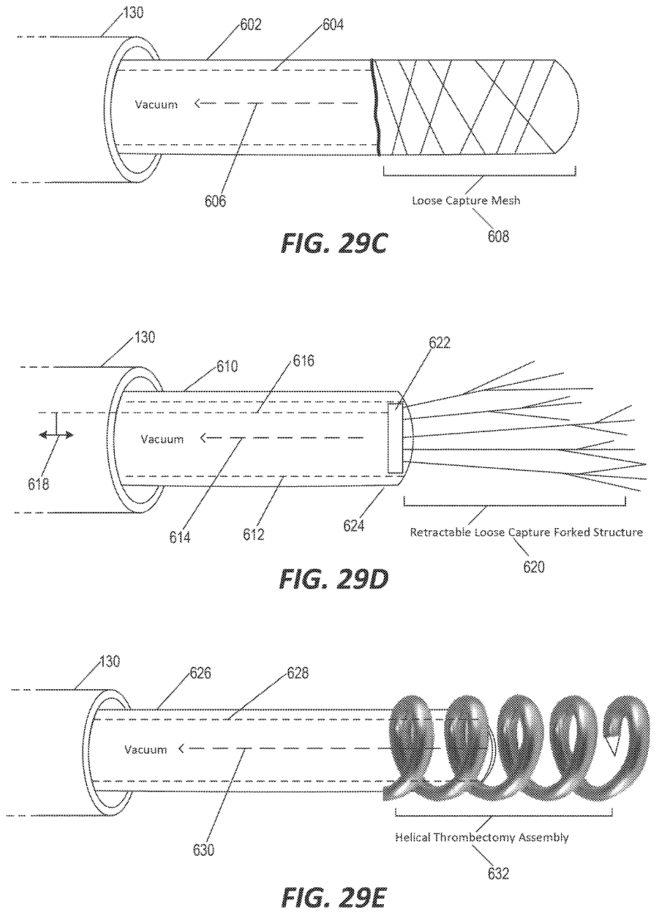

Referring to FIG. 29A, a scissor tool (570) assembly is shown advanced through an elongate instrument (130). The scissor tool or probe (570) may comprise a substantially fixed portion (572) movably coupled to a rotatable portion (574) which may be controllably rotated open or shut along an elongate cutting or dissecting margin (584) to provide controllable scissor-like functionality. The rotatable portion (574) may be rotatably coupled at a joint (582) and coupled (578) to a control member (576; such as a tension or compression rod or wire) which may be extended proximally to provide for controlled insertion or retraction (578), such as via an operator manually or electromechanically (such as via a motor and capstan configuration).

Referring to FIG. 29B, a grasper tool body (596) is shown positioned through an elongate instrument (130) fixedly coupled to a first distal grasper member (586) and rotatably coupled (via a joint 600) to a rotatable second distal grasper member (588), the proximal portion of which is coupled (598) a control member (592; such as a tension or compression rod or wire) which may be extended proximally to provide for controlled insertion or retraction (594), such as via an operator manually or electromechanically (such as via a motor and capstan configuration), to create controlled grasping and ungrasping between (590) the distal grasper members (586, 588).

Referring to FIG. 29C, a probe or tool (602) is positioned through an elongate instrument (130) featuring a loose and porous capture/agitation mesh (608) along with an internal vacuum lumen (604) for vacuum flow (606). The capture/agitation mesh (608) may be configured to capture, adhere to, dissolve, and/or reduce certain targeted tissue structures or other structures, such as plaques or clots, such that they, or portions thereof, may be captured and/or vacuum-transported more proximally, such as into the vacuum lumen (616) of the tool (610).

Referring to FIG. 29D, a probe or tool (610) is positioned through an elongate instrument (130) featuring a loose and porous branched or forked capture/agitation geometry (620; may be retractable, and may be configured to be self-expanding upon release out of the distal end of the tool 610, and otherwise compactly restrained when pulled into or held within the inside of the working lumen 612 of the tool 610). The working lumen (612) of the tool may also be utilized as a lumen for vacuum flow (614). The branched or forked capture/agitation geometry (620) may be configured to capture, adhere to, dissolve, and/or reduce certain targeted tissue structures or other structures, such as plaques or clots, such that they, or portions thereof, may be captured and/or vacuum-transported (614) more proximally, such as into the working lumen (612) of the tool (610). In a retractable configuration, the branched or forked capture/agitation geometry (620) may be proximally coupled to a controllably movable coupler member (622) which is coupled to a repositioning control member (616; may be a push or pull rod or wire, for example; may extend proximally to provide for controlled insertion or retraction (618), such as via an operator manually or electromechanically (such as via a motor and capstan configuration); the repostioning control member (616) may be utilized to advance out, or retract in, the branched or forked capture/agitation geometry (620).

Referring to FIG. 29E, another probe instrument (626) embodiment is illustrated placed through an elongate instrument (130) having a helical thrombectomy assembly (632) operatively coupled with a vacuum lumen (628) configured to have vacuum flow (630) controllably available to pull proximally any items, such as tissue portions or portions of other structures such as clots, plaques, or other structures, which may be encountered at the helical thrombectomy assembly (632), which may be rotated or inserted relative to the elongate instrument (130), or together with rotation or insertion of the elongate instrument (130), to provide for engagement with such other structures.

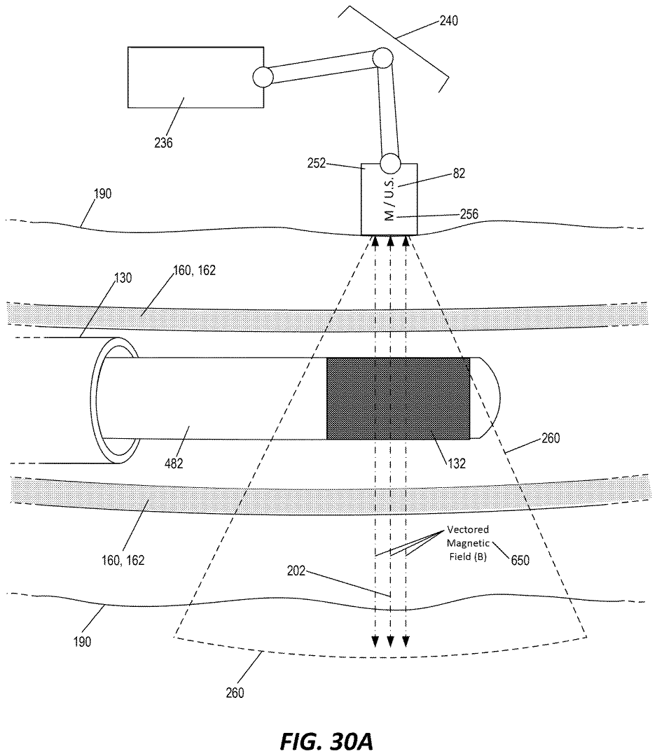

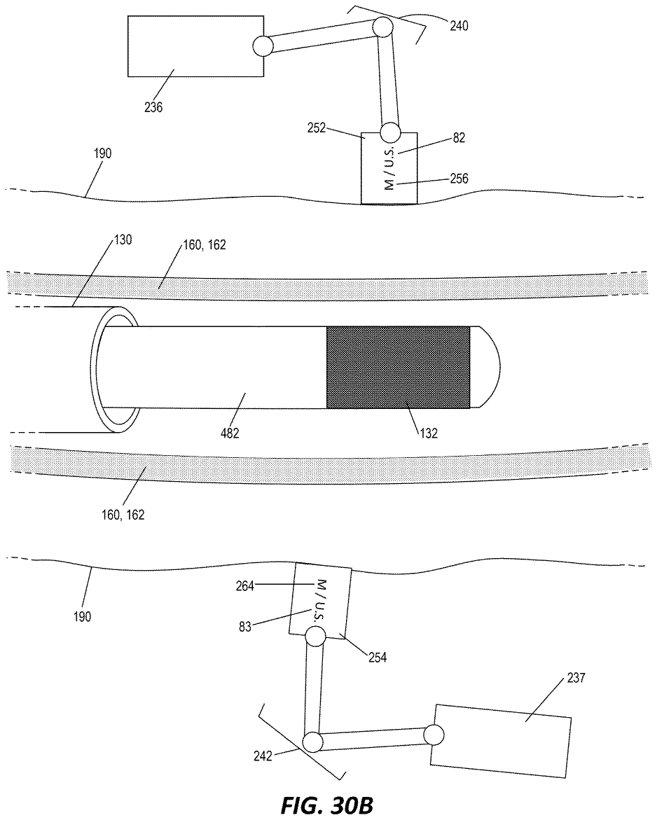

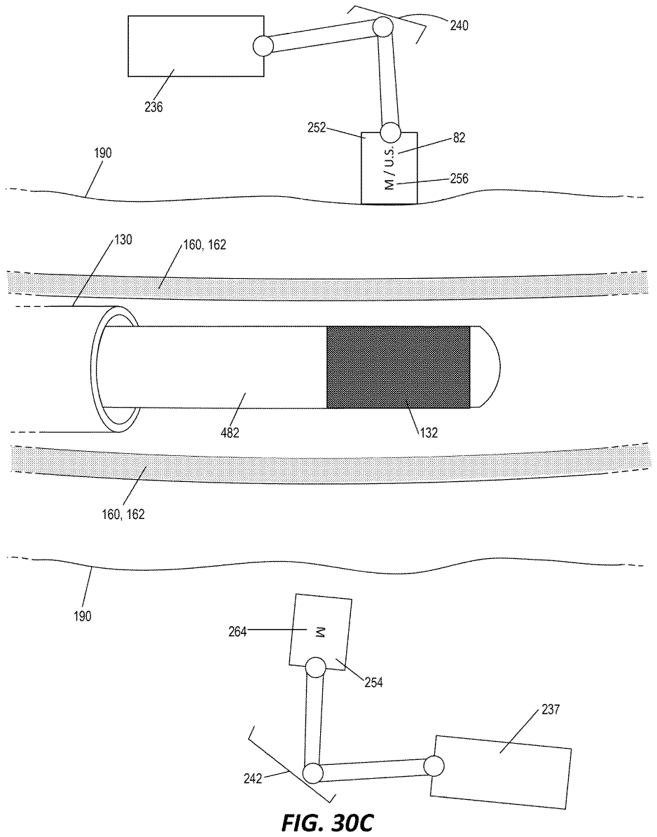

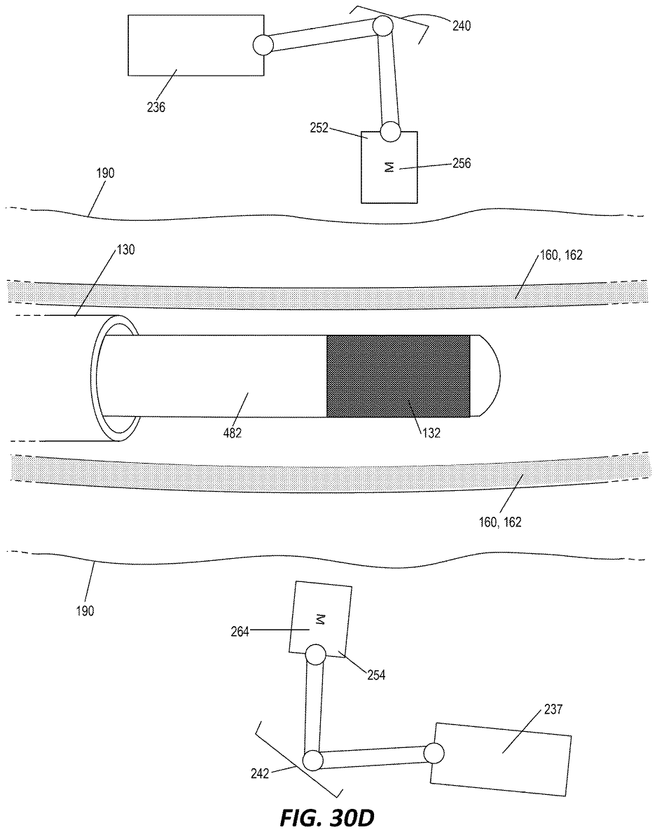

Referring to FIG. 30A, as noted above, such as in reference to FIG. 20B, a movable arm, such as a robotic arm (240) may be utilized to controllably position and orient an end effector assembly (252), such as one configured to carry both an ultrasound transducer (82) and a magnetic field element (256), and to urge at least the ultrasound transducer (82) against the skin or other tissue structure (190) of a patient to facilitate image capture withing a field of view (260) for the transducer (82). The magnetic field element (256) may be configured to produce a magnetic field or magnetic flux (650) which may be vectored (202) and applied to produce a steering load on a ferromagnetic steerability element (132) which may comprise a portion of an probe, tool, or instrument (482), which may itself be threaded through a working lumen of a steerable elongate instrument (130) as shown. Such a configuration may be utilized to navigate aspects of a lumen (160) such as a blood vessel (162) of a patient to conduct certain aspects of an intervention.

Referring to FIG. 30B, a configuration similar to that of FIG. 30A is illustrated, with the addition of a second robotic arm (242) and end effector assembly (254) which may be controllably positioned and oriented to apply a separate and potentially overlapping magnetic field (264) or magnetic flux to, for example, provide additional superimposed loading upon a targeted ferromagnetic steerability element (132). The second end effector assembly (254) is shown also comprising an additional ultrasound transducer (83) capability for additional imaging which may be at least partially redundant with the imaging provided by the first ultrasound transducer (82) shown in opposing positioning and orientation. The configuration of FIG. 30C illustrates a configuration similar to that of FIG. 30B, but without the additional ultrasound capability on the second end effector assembly (254), and thus it is not a critical for the second end effector assembly to be in direct contact (i.e., ultrasonic transmission contact) with the nearby portion of the tissue structure (190). FIG. 30D illustrates a configuration wherein neither the first (252) nor second (254) end effector assemblies are carrying an ultrasound transducer, but both are able to apply and vector magnetic fields for steerability influencing of the targeted ferromagnetic steerability element (132).

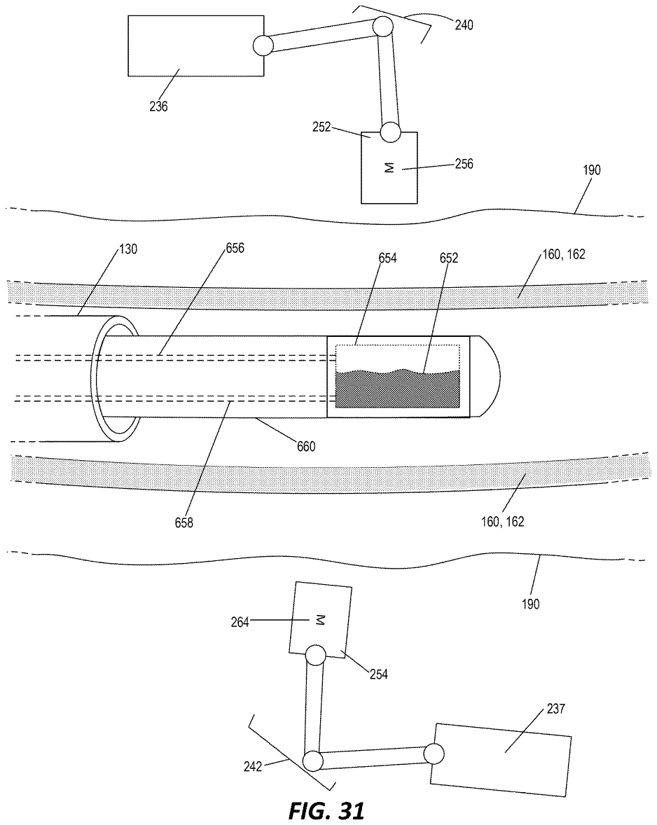

Referring to FIG. 31, a configuration similar to that of FIG. 30D is illustrated, with the addition of a flowable ferromagnetic fluid (652) which may be flowed or exited through lumens (656, 658) formed in the instrument leading back proximally to a control point wherein modulation of an amount of the ferromagnetic fluid (652) in a distally-positioned reservoir (654) may be controllably modulated to modulate steerability under magnetic flux or magnetic field (256, 264). To enhance or increase steerability load per given magnetic flux applied, greater ferromagnetic fluid (652) may be positioned within the distal reservoir (654).

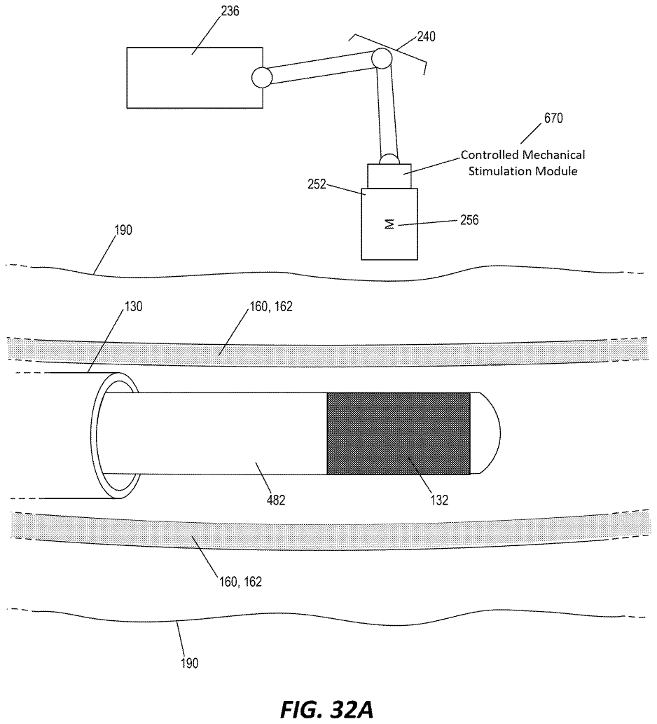

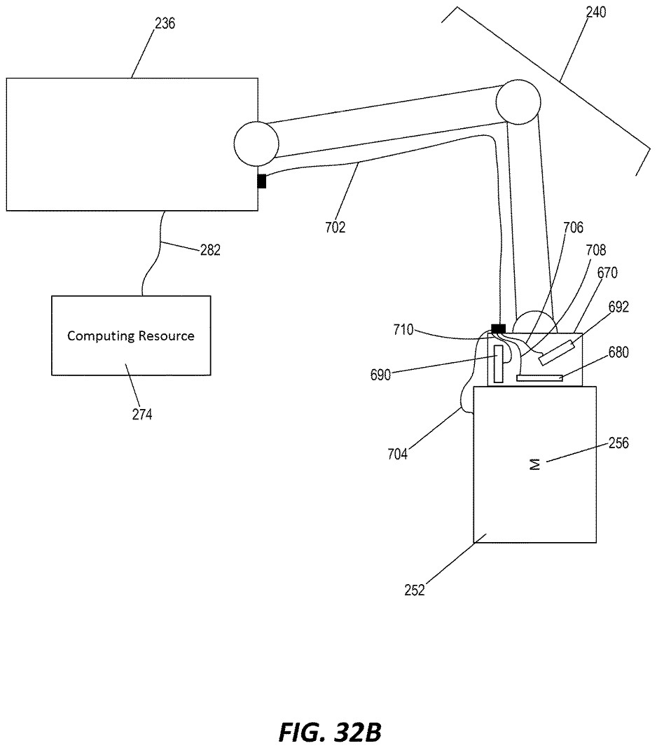

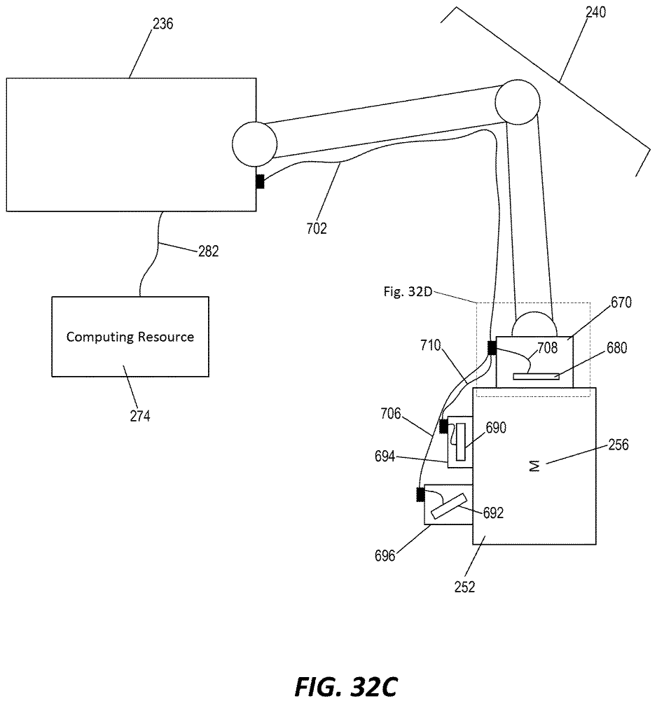

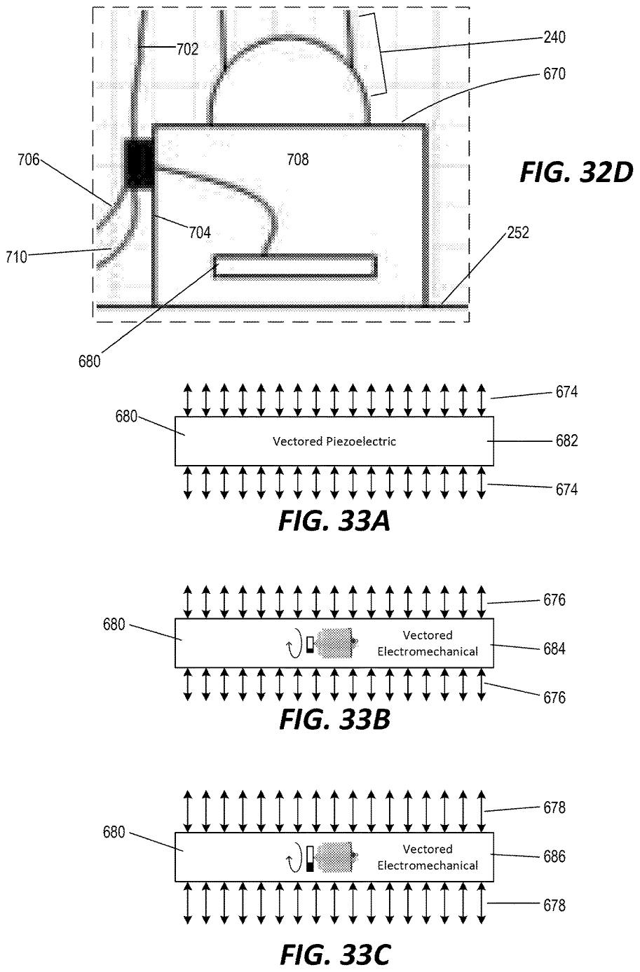

Referring to FIGS. 32A-37B, in various clinical scenarios, it may be useful and/or desirable to induce controlled oscillation to a working or distal end of an instrument. For example, it may be useful or desirable in dissolving, breaking up, removing, or otherwise disrupting a structure such as an embolism, clot, thrombus, or plaque to induce oscillatory motion to the working instrument which has a specified amplitude at a specified vector, as well as a specified frequency of oscillation. It also may be preferrable to have the oscillatory motion induced from outside of or adjacent to the instrument, rather than from within the instrument (i.e., in comparison to a configuration wherein an on-board oscillator such as a piezoelectric transducer might potentially be utilized to induce oscillatory motion), to assist in controllability, geometry, and power transfer, for example.

Referring to FIG. 32A, a configuration similar to that of FIG. 30D is shown, without the second assembly of end effector magnetic element (264) and associated robotic arm (242) and robot stabilization base (237). As described above, such a configuration presents a steerable elongate instrument (130; magnetic element not shown in FIG. 32A, but refer, for example, to FIGS. 13A, 15A, 19A above featuring more detailed views of elongate instrument 130 configurations) with a steerable probe or tool (482) positioned therethrough, the probe or tool (482) comprising a magnetic element (132) which may be utilized for steerability of the probe or tool (482) under the influence of magnetic field or magnetic flux applied by the controllably applied and vectored end effector magnetic element (256).

In practice, it may be useful to controllably steer and navigate (such as via magnetic influence) the outer steerable elongate instrument (130) through the body and into a position adjacent an interventional target, then leave such outer steerable elongate instrument (130) in place, while subsequently advancing a working tool such as that (482) shown in FIG. 32A toward the interventional target with magnetic steering thereof for the intervention. In other words, a sequence of navigation and steerability of instruments may be desired, as opposed to parallel processing and navigation, to simplify complexities such as magnetic flux superimposition, multi-robot operation, ferromagnetic local nonlinearities, and other factors which may be more prevalent with a parallel type of navigation configuration.

Referring again to FIG. 32A, a controlled mechanical stimulation module (670) may be coupled to the end effector magnetic element (256) and configured to induce precision prescribed oscillatory motion to the effector magnetic element (256), so that the magnetic flux or field emitted by the effector magnetic element (256) oscillates as well, to produce oscillatory motion induction of the impacted ferromagnetic element (132).

Referring to FIG. 32B, a closer view of a controlled mechanical stimulation module (670) is illustrated with three orthogonally-oriented oscillation-inducing modules (680, 690, 692) operatively coupled (such as by wired 708, 710, 706, respectively; or wirelessly connected such as via IEEE 802.11 transceivers, or Bluetooth™, nearfield, or other wireless transceivers) back (702, 282) to the computing resource (274) for precision control and activation. The configuration of FIG. 32B shows each of the orthogonally-oriented oscillation-inducing modules (680, 690, 692) in a common housing; the configuration of FIG. 32C shows each of the orthogonally-oriented oscillation-inducing modules (680, 690, 692) in separate but coupled housings.

FIG. 32D illustrates a close-up view of a portion of FIG. 32C with a view of one of the orthogonally-oriented oscillation-inducing modules (680) centered, and FIGS. 33A-33C illustrate functional diagrams featuring various different configurations for creating controlled oscillation within such orthogonally-oriented oscillation-inducing module (680). For example, referring to FIG. 33A, a piezoelectric transducer (682) may be utilized for controlled oscillatory excitation (674) with a prescribed amplitude in a prescribed vector at a prescribed frequency. FIG. 33B illustrates that an electric motor with offset (i.e., intentionally “lopsided”) rotatable member may be utilized in an oscillation-inducing module (684) for controlled oscillatory excitation (676) with a prescribed amplitude in a prescribed vector at a prescribed frequency. With a larger or more lopsided or offset rotatable member in an oscillation-inducing module (686), oscillatory motion (678) factors such as amplitude and frequency may be tuned, as shown in FIG. 33C.

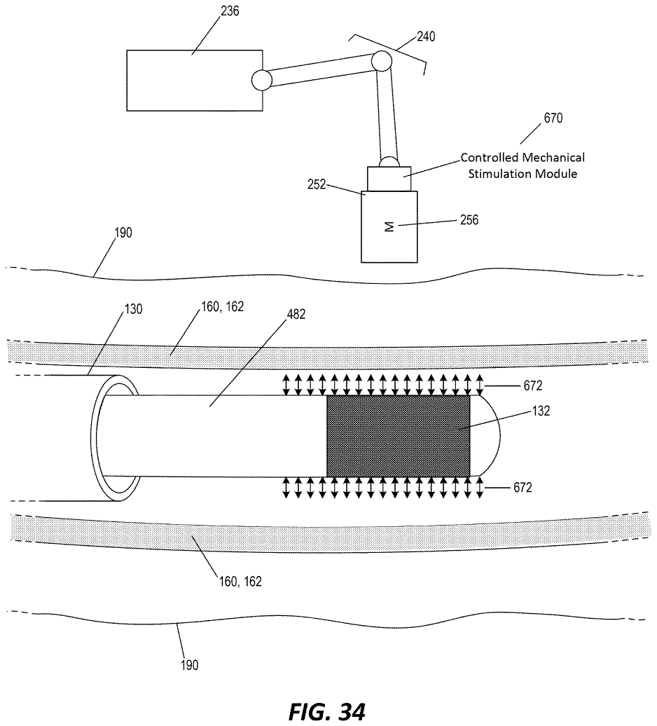

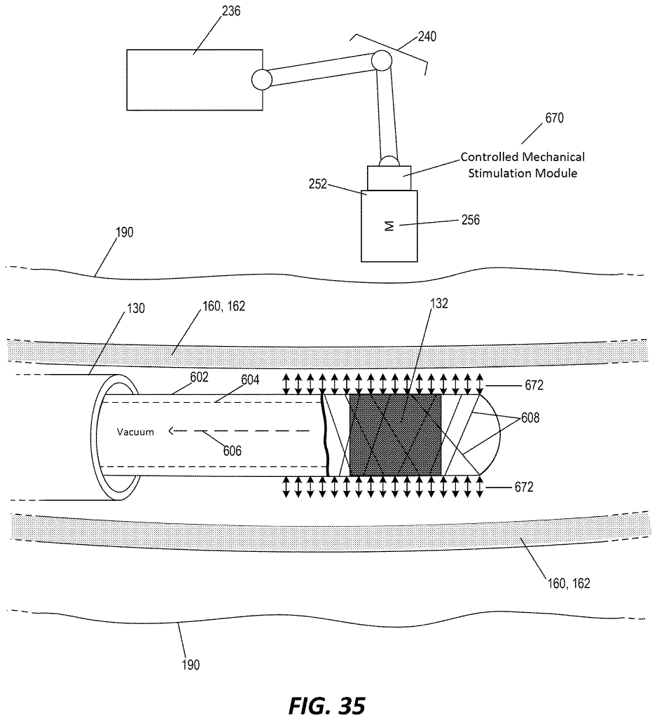

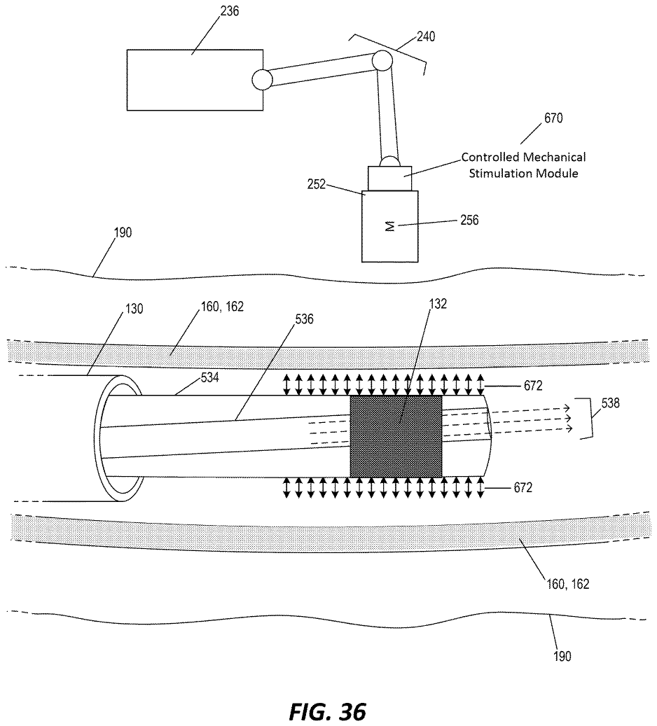

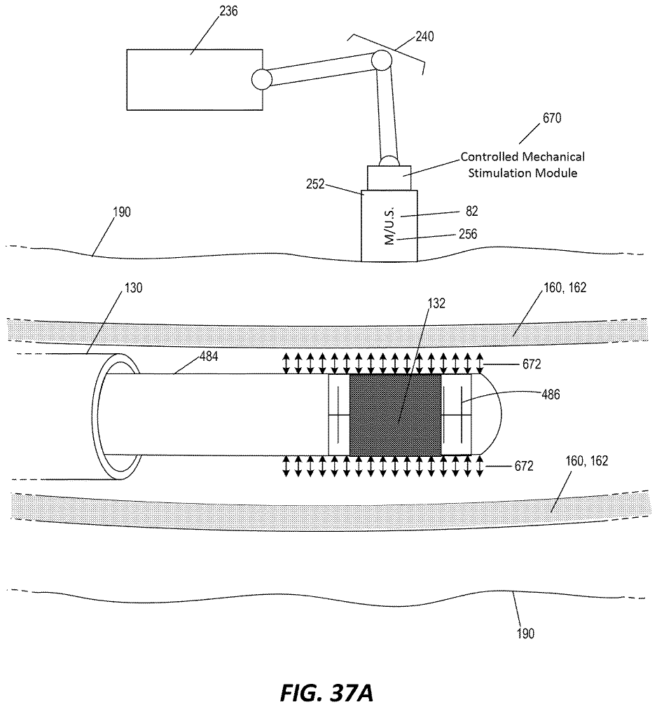

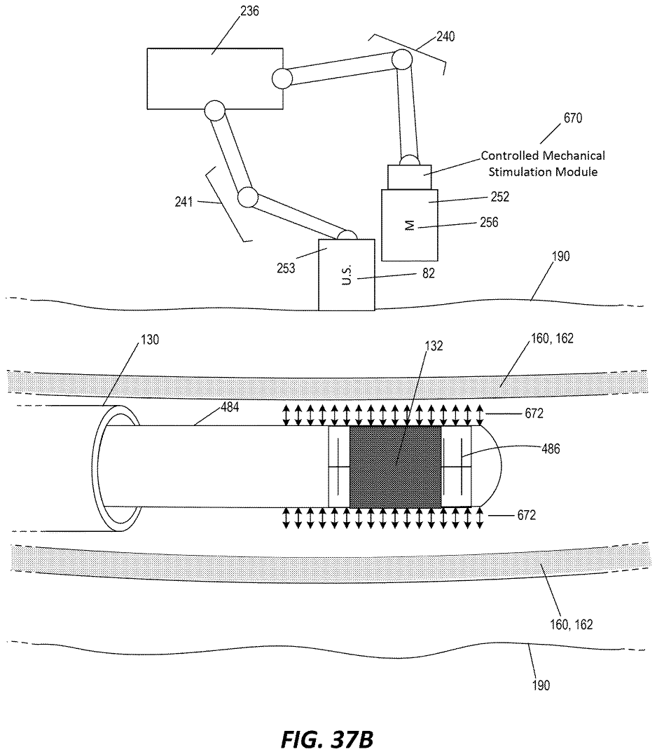

Referring to FIGS. 34-37B, in practice, controlled oscillatory motion may be utilized, for example, to dislodge portions of tissue structures or other objects, such as portions of plaques, clots, or other structures. For example, FIG. 34 illustrates a configuration similar to that of FIG. 32A, but with the controlled mechanical stimulation module (670) inducing controlled oscillatory motion (672) of the ferromagnetic element (132) and associated probe (482). FIG. 35 illustrates a configuration similar to that of FIG. 29C, with the addition of a ferromagnetic element (132) and induction of controlled oscillatory motion (672) of the ferromagnetic element (132) and associated probe (602). Such an assembly may be utilized, for example, in addressing, disrupting, and or dislodging portions of tissue structures or other objects, such as portions of plaques, clots, or other structures. For illustrative purposes, FIG. 36 illustrates a configuration similar to that of FIG. 28B, with the addition of a ferromagnetic element (132) and induction of controlled oscillatory motion (672) of the ferromagnetic element (132) and associated probe (534), to facilitate application of an oscillatory perfusion (538). FIG. 37A illustrates a configuration similar to that of FIG. 26C, with the addition of a ferromagnetic element (132) and induction of controlled oscillatory motion (672) of the ferromagnetic element (132) and associated probe (484), to facilitate application of an oscillatory resonance, or so-called compound resonance (i.e. oscillating from both resonance and magnetic field induced oscillation) from the resonance assembly (486; here shown configured to be resonantly stimulated by applied ultrasonic radiation from the ultrasound transducer 82); such a configuration may be useful in controlled agitation, removal, and/or disruption of various targeted tissue structures or objects, such as plaques or clots. FIG. 37B shows a configuration similar to that of FIG. 37A, to illustrate that the end effector magnetic element (256) may reside on a separate robotic arm (240) relative to the ultrasound transducer (82) and its associated robotic arm (241) for additional magnetic field vectoring options during ultrasound application through the transducer (82).

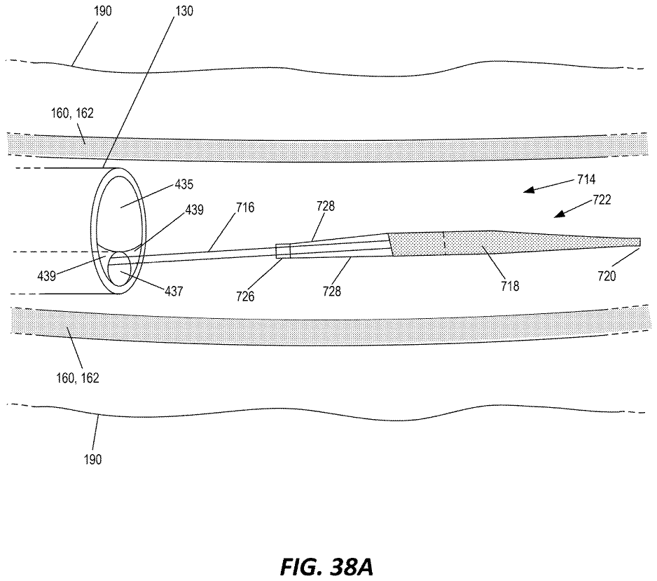

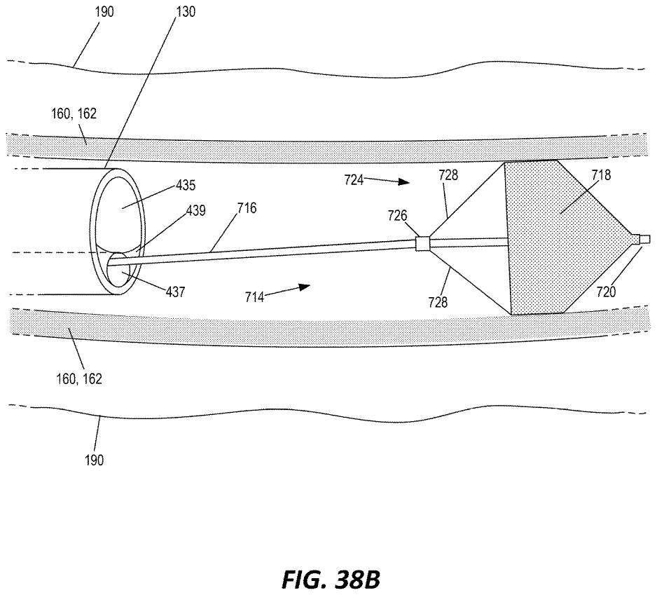

Referring to FIGS. 38A and 38B, in various embodiments, it may be desirable to provide distal protection to prevent various nearby or dislodged materials, objects, or portions thereof from escaping the local interventional environment and volume and escaping farther distally where they may not easily be recovered. For example, in a stroke intervention or other cardiovascular interventional clinical scenario, it may be valuable to prevent any embolic, thrombus, or clot material from moving away from a zone of intervention. This may be accomplished and/or assisted through the use of a distal protection configuration such as that illustrated in FIGS. 38A-38B, wherein a distal protection instrument assembly (714) may be inserted (using an intercoupled elongate shaft member 716) through an elongate instrument (130), such as through one (437) of a plurality of working lumens, into a lumen (160) or vessel (162) within a tissue structure (190) of a patient in a collapsed configuration (722) as shown in FIG. 38A. As shown in FIG. 38B, the assembly (714) may be configured to be self-expanding (such as when pulled proximally after being inserted distally) or controllably expandable (such as via applied tension or compression on a control member which may be coupled to an element such as the proximal coupler 726) to an expanded configuration (724) wherein an expandable capture mesh (718) substantially fills the lumen (160) or vessel (162) and tapers to a distal portion (720) to effectively provide a removable capture net or filter which may be configured to allow normal passage of blood or other fluid, but not clots, plaques, or other larger structures. The expandable capture mesh (718) may be supported by a plurality of coupling members (728) coupled to the coupling member (726).

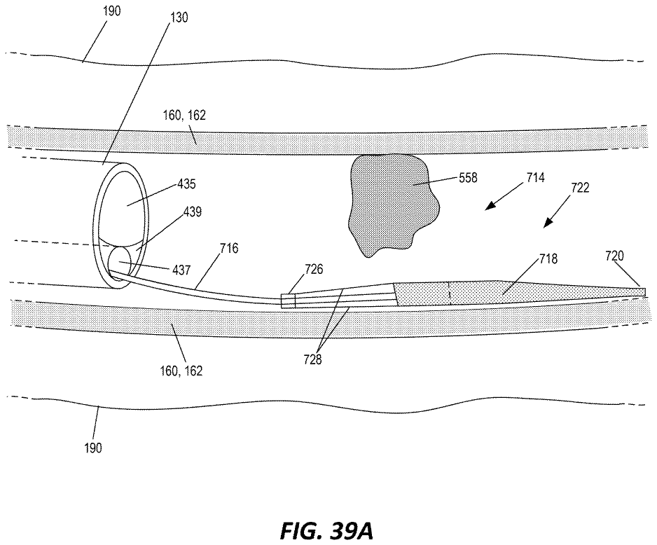

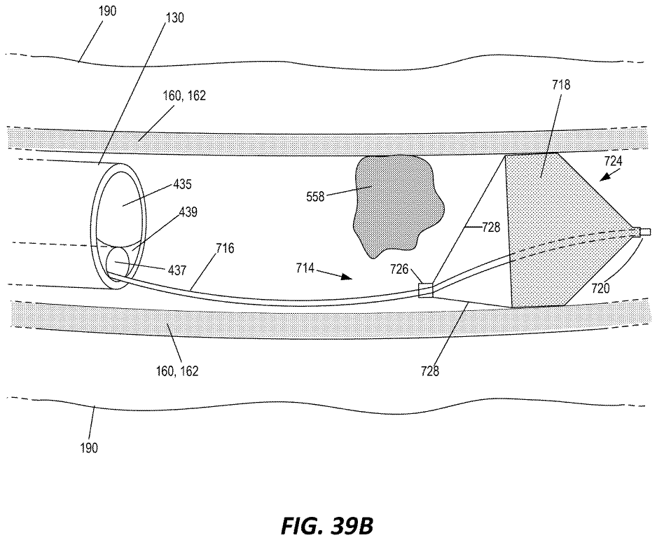

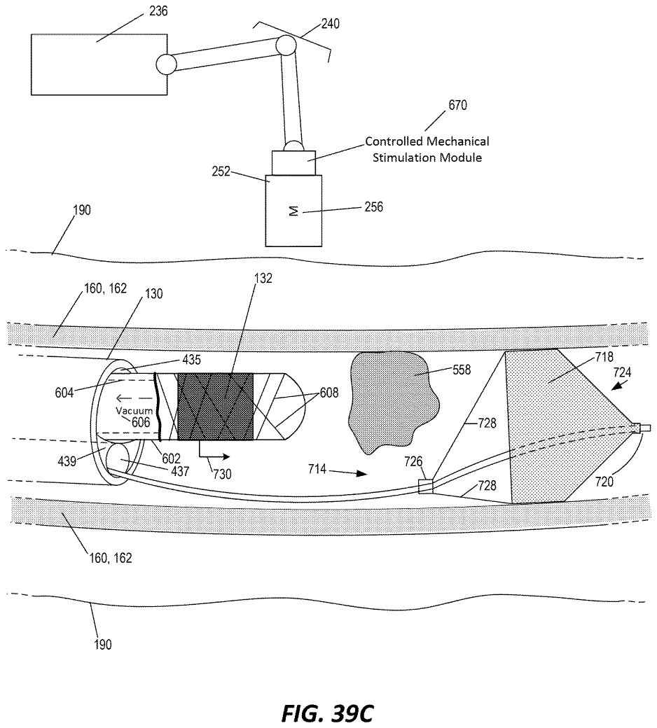

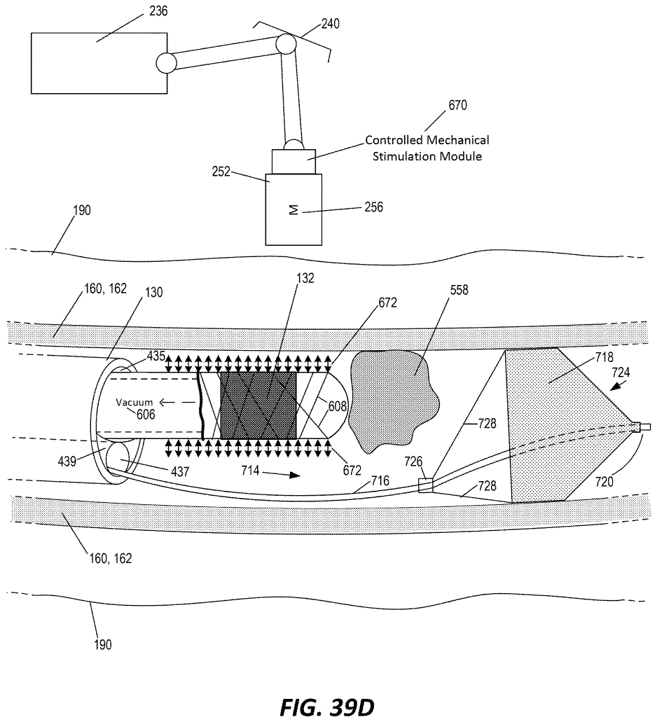

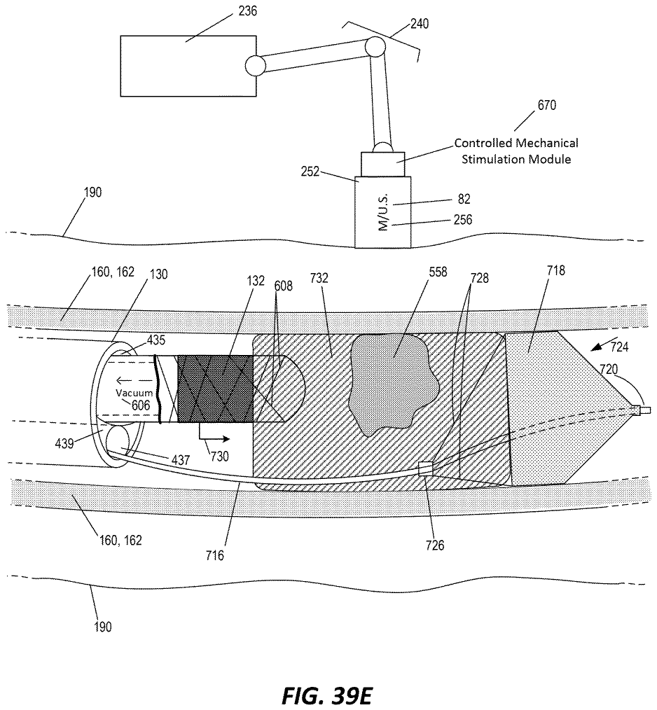

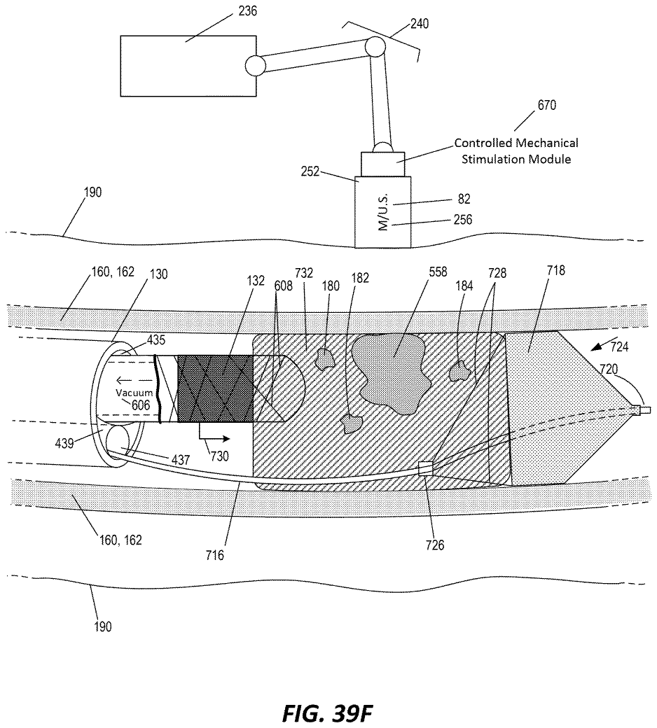

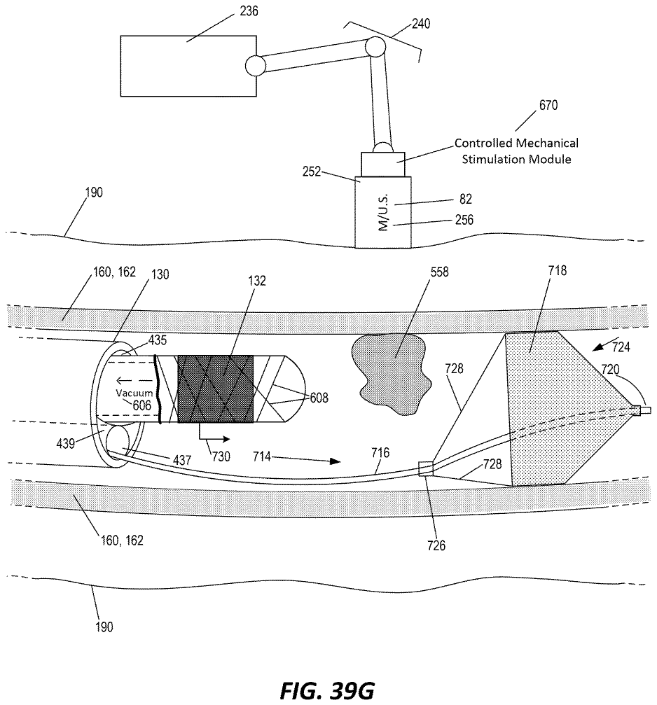

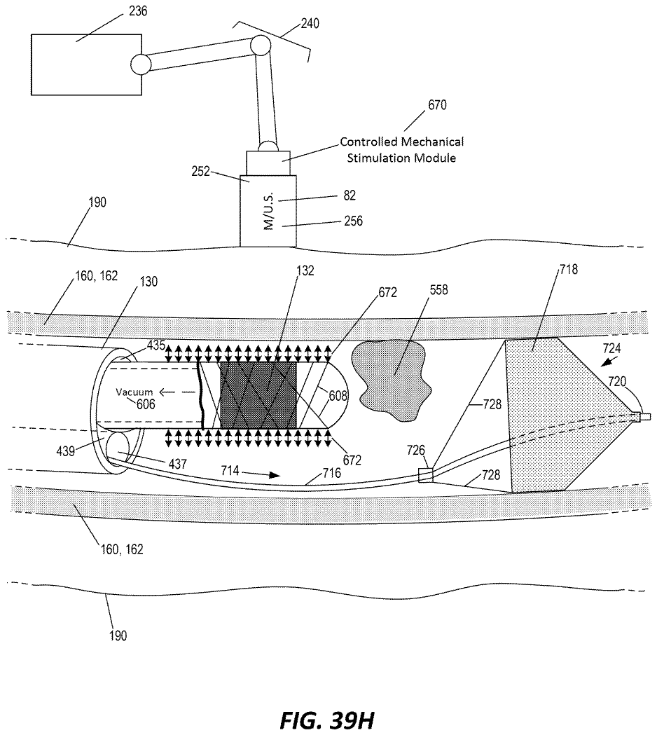

Referring to FIGS. 39A-39H, an illustrative clinical scenario is shown featuring various of the aforementioned configurations and variations. Referring to FIG. 39A, a clot, thrombus, or embolism (558) is shown positioned within a lumen (160) such as a blood vessel (162) of a tissue structure (190) of a patient. Should a clinical operator decide that it is indicated to attempt to remove the clot (558) after preoperative imaging and analysis, an elongate instrument (130) may be advanced within a near proximity, such as within a few inches, of the clot (558) using steering configurations such as those described above in reference to FIG. 20B, for example. Once in such position, the elongate instrument (130) may be at least temporarily utilized as a stable base of operation for other instrumentation, such as an interventional probe or other tool, and/or a distal protection device or assembly (714), such as that shown in FIG. 39A being advanced past the clot (558) in an intentionally collapsed configuration (722) so that it may be passed beyond the clot (558) before controlled expansion to an expanded state (724) as shown in FIG. 39B. With the expandable capture mesh (718) safely deployed and confirmed, such as via fluoroscopy, ultrasound, and/or radiography, for example, further interventional steps may be conducted in furtherance of removing and/or disrupting the clot (558).

Referring to FIG. 39C, a configuration similar to that described in reference to FIG. 35 is illustrated, having been advanced (730) through a working lumen of the elongate instrument (130) with the distal protection assembly (714) already in place in the expanded state (724). Referring to FIG. 39D, the tool (602) may be controllably advanced, such as under observation by fluoroscopy and/or ultrasound and with steerability and navigation assisted by magnetic flux or magnetic field (256), to be within proximity of the clot (558) such that the controlled mechanical stimulation module (670) may be utilized to cause controlled and selected oscillatory motion (672) to assist in disrupting the clot (558) if vacuum (606) alone hasn't encouraged the clot (558) or portions thereof to already exit the vicinity through the working instrument (602). Referring to FIG. 39E, it may also be desirable before or after attempting to remove the clot using vacuum (606; i.e., before attempting mechanical agitation using controlled oscillatory motion 672) to perfuse the local volume around the clot (558) with various fluids (732), such as contrast agent to enhance visibility with associated imaging modalities (such as fluoroscopy and/or ultrasound), and/or to assist in the disruption or break-up of the clot via radiation-enhanced means (for example, in one embodiment, microbubbles may be perfused into the volume around the clot 558 so that applied radiation, such as via ultrasound 256, may create cavitation and/or shockwaves around the clot 558 to assist in disruption; in another embodiment compounds may be at least temporarily introduced to chemically enhance likelihood of clot breakup, disruption, and/or detachment). FIG. 39F illustrates a configuration similar to that of FIG. 39E, with the clot (558) at least partially disrupted into one or more clot portions (180, 182, 184) which preferably may be maintained within proximity of the vacuum (606) capability for removal by vacuum (606), or which may alternatively be captured by the expandable capture mesh assembly (718) of the distal protection assembly (714).

FIG. 39G illustrates a configuration similar to that of FIG. 39F, after vacuum has removed the perfusant (732) and smaller clot portions (180, 182, 184). To facilitate removal and/or disruption of the remaining relatively large clot portion (558), the probe (602) may be advanced (730), as described above, to be proximity of the clot (558) so that the distal portion of the instrument (602) (shown comprising a loose capture/agitation mesh assembly 608) may be utilized, such as with applied oscillatory motion (672) as in FIG. 39H, to disrupt and preferably remove (such as via vacuum 606 and/or adhesion to the mesh assembly 608) the remaining clot (558) material, after which the probe (602) may be retracted toward the elongate instrument (130), along with the distal protection assembly (714) which ultimately may be returned to a collapsed configuration (722) for removal.

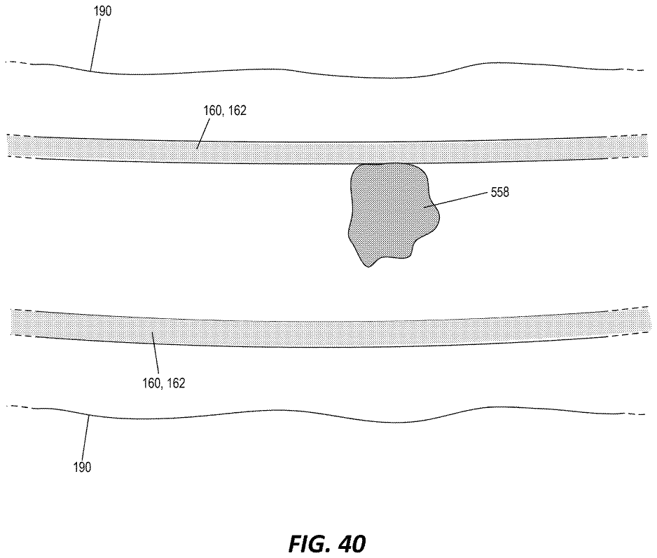

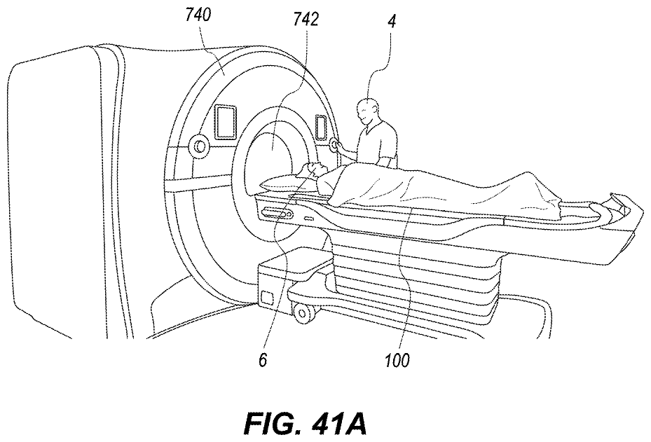

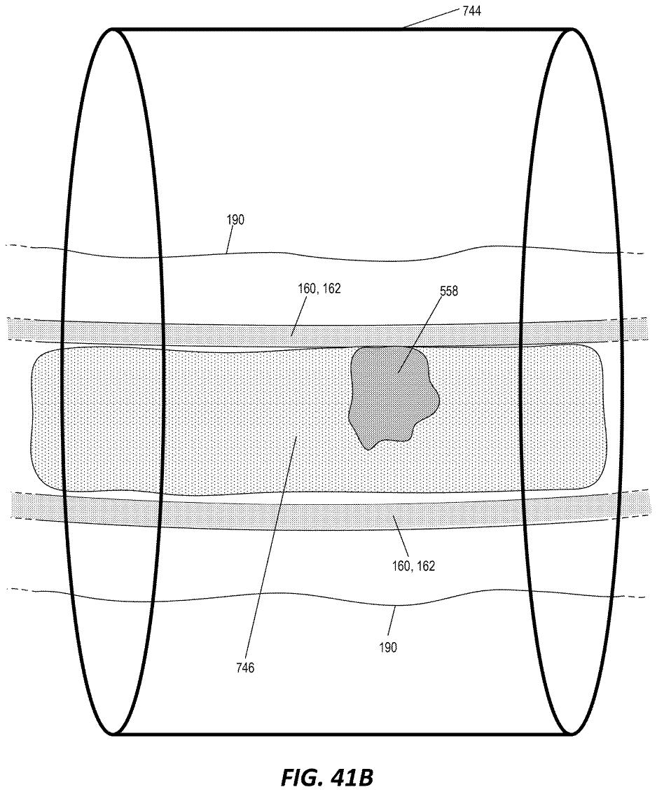

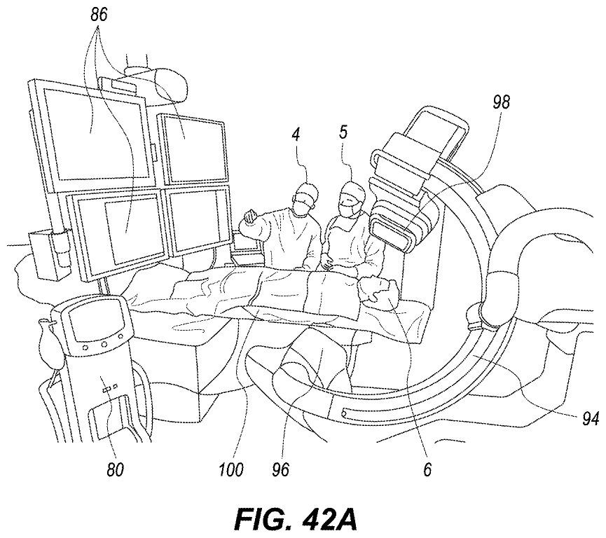

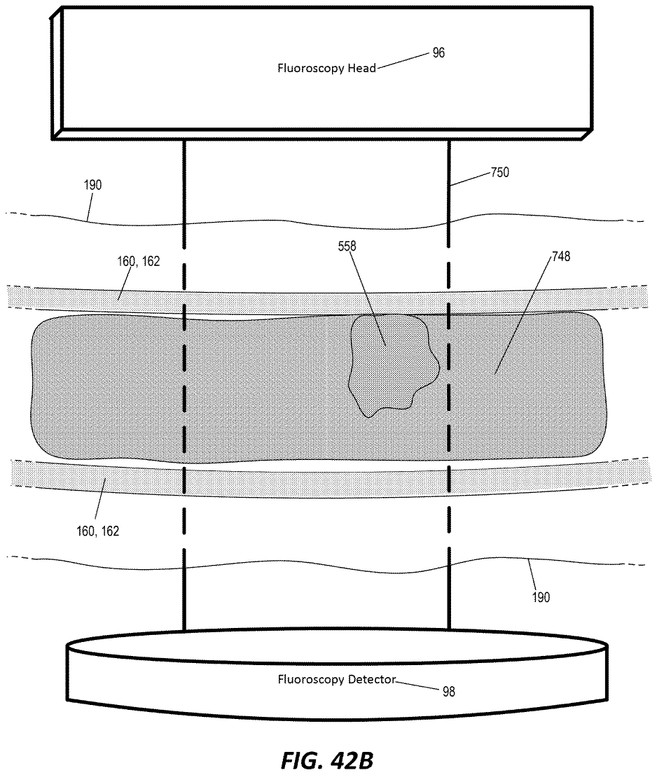

Referring to FIGS. 40-51F, another illustrative clinical scenario is shown featuring various of the aforementioned configurations and variations. Referring to FIG. 40, a clot (558) is positioned within a lumen (160) or vessel (162) of a tissue structure (190) of a patient. Referring to FIG. 41A, to analyze the patient (6), image data capture and analysis may be conducted, such as via a magnetic resonance imaging (“MRI”) system (740) comprising an aperture (742) into which a hospital bed (100) supporting the patient (6) may be inserted during scanning. For conditions such as suspected stroke, certain MRI systems and scanning paradigms have been developed to be relatively expedient in time given the potential impact of any delay. Referring to FIG. 41B, an MRI scanning session may result in image capture data in the form of slices, assembled pixels or voxels, and/or solid models, for example. A field of capture (744) may have a roughly cylindrical or other shape, and may contain image data pertinent to structures such as skin or organ margins, lumens (160) or vessels (162), and/or structures such as clots, thrombi, or embolisms (558), which may be highlighted in the image data via use of certain contrast agent compounds (746) which may be delivered intravascularly to the approximate location of the suspected clot, thrombus, or embolism (558). Referring to FIG. 42A, fluoroscopy may also be conducted to further analyze the clinical scenario. A patient (6) is shown on a table (100) positioned between fluoroscopy transmitter head (96) and receiver/detector (98) components which may be coupled to a so-called “C-arm” structure (94). Image information may be displayed on the associated intercoupled monitors (86) from fluoroscopy as well as from an ultrasound system (80) which may be coupled to one or more of the displays (86) and/or an intercoupled computing system; fluoroscopy and ultrasound scanning may be conducted simultaneously and each may be optimized with various intravascular compounds such as contrast agents which may be injected or perfused to targeted tissues of interest, surrounding blood, or other fluids. FIG. 42B illustrates a schematic of a fluoroscopy scenario wherein a transmission head (96) transmits radiation through the patient's tissue structures (190, 160, 162, 558) to the detector (98); element 748 is representative of contrast agent which may be delivered to the region to assist in the image capture and development.

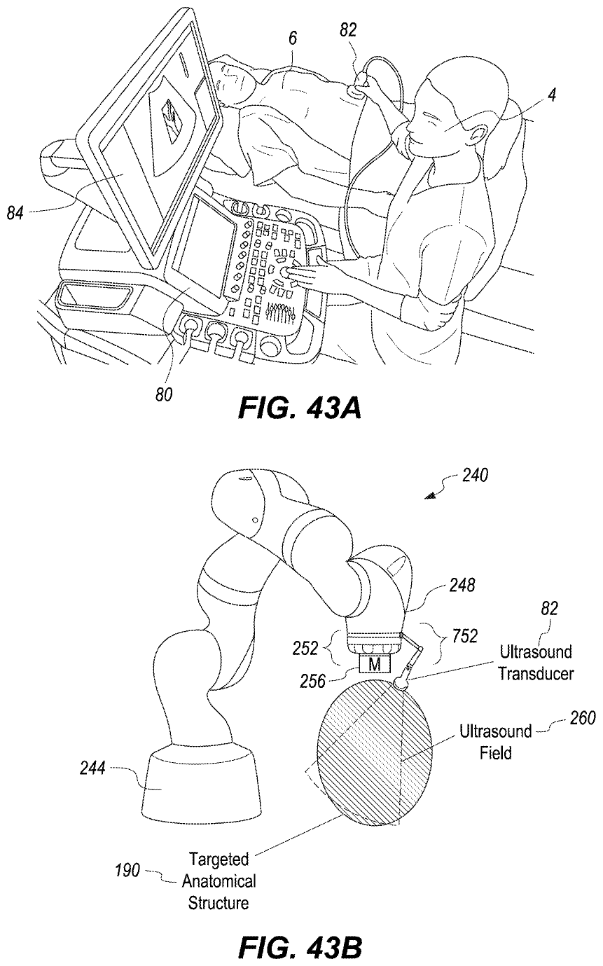

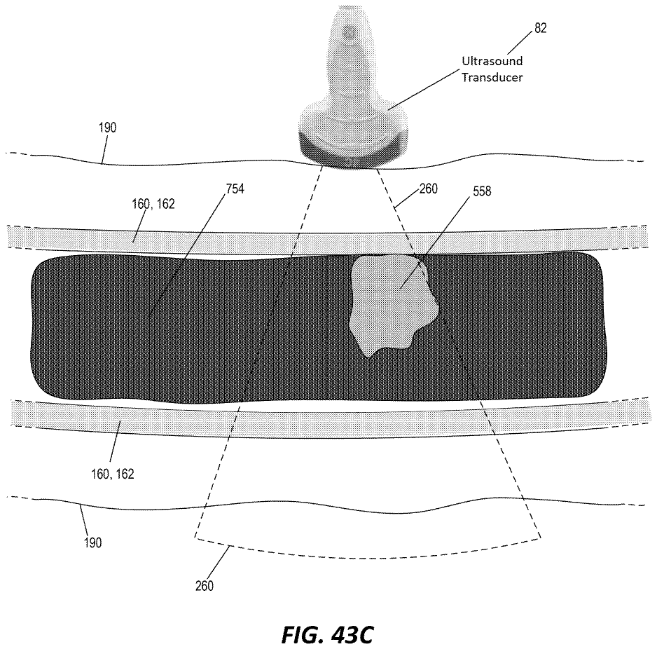

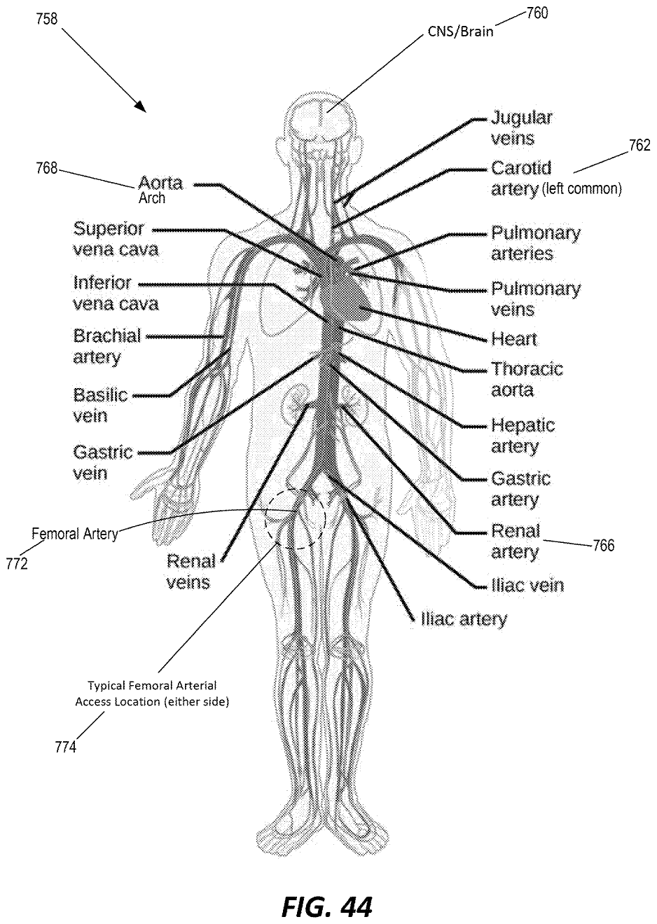

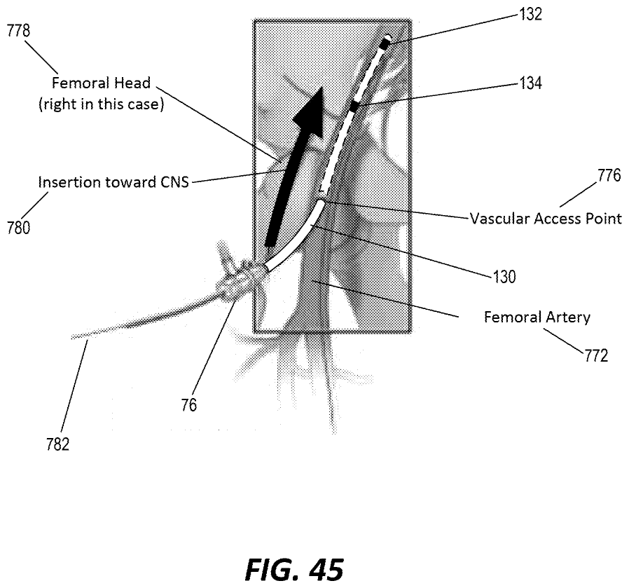

Referring to FIG. 43A, ultrasound imaging may be conducted before or after other modalities (i.e., such as fluoroscopy or MRI), in addition to simultaneously with such modalities. FIG. 43A illustrates an operator (4) utilizing an ultrasound transducer (82) manually on a patient (6) to examine certain targeted tissue structures which may be observed using the ultrasound processing system (80) and display configuration (84). Referring to FIG. 43B, as discussed above, a robotic arm (240) may be utilized to precisely position and orient an end effector assembly (252), which may be coupled to an end effector magnetic element (256), and/or other end effector elements such as an ultrasound transducer (82), here shown coupled to the end effector assembly (252) utilizing an adjustable vectoring assembly (752) which may be manually, or electromechanically movable (for example, may comprise a robotic arm or electromechanically controllable joint, or plurality thereof) to position and orient the ultrasound transducer (82) and associated field of view or field of capture (260) optimally relative to the patient tissue structure (190) and also relative to the other control components such as the end effector magnetic element (256). FIG. 43C illustrates a schematic of an ultrasound scenario wherein the ultrasound transducer (82) is positioned against the tissue structure (190) of the patient and vectored such that the field of view (260) at least partially captures targeted tissue structures, such as portions of a lumen (160) or vessel (162), a clot (558), and/or other nearby structures. An ultrasound contrast agent (754) may be delivered to the area, such as by intravascular injection, to assist in the ultrasonic imaging. Referring to FIG. 44, after a clinical decision that intravascular intervention is indicated and a particular anatomical location is to be targeted for access, an interventional route must be selected. For example, in the case of a clot residing in the neurovasculature of the central nervous system, the medical team may decide to gain access through the blood vessels, starting at a location such as the femoral artery (772) via surgical access (also called surgical “cut down” or use of the Seldinger Technique, wherein a small access point may be dilated using needles and/or guidewires to ultimately provide access for a tubular member such as a catheter). A typical access location or zone (774) to gain access to the femoral artery (772) is illustrated in FIG. 44. Referring to the close-up view of FIG. 45, a particular cut-down or Seldinger access point (776) may be selected, and access provided for a catheter member such as an elongate instrument (130) as described herein; such instrument (130) may be passed through proximal instrument assembly such as a Luer assembly, which may comprise a valve and/or fluid perfusion port; a guidewire (782) also may be utilized to assist with gaining initial access (i.e., as in a Seldinger access configuration), and/or may be utilized to assist with guiding a tubular member or assembly such as an elongate instrument (130) in what is known as an “over-the-wire” configuration wherein incremental advancements of a guidewire through the subject anatomy may be followed by incremental advancements of the instrument over-the-wire (i.e., using the wire as a physical guiding member). FIG. 45 illustrates general directional advancement (780) of the elongate instrument (130) toward the central nervous system, past other anatomy such as the skeletal femoral head (778) of the patient. During each stage of advancement, from initial vascular access point (776), past the initially encountered portions of the femoral artery (772), and toward the central nervous system, magnetic field or flux may be controllably applied and vectored (such as described above in reference to FIG. 20B employing one or more robotic arms) to influence the ferromagnetic elements (132, 134) to assist in navigating the elongate instrument (130), preferably in a manner in accordance with a plan and selected preferences, such that it may generally follow a prescribed plan and prioritize movement and loading in accordance with operational priorities such as prescribed keep-away zones or locations, known singularities or navigational issues, and the like.

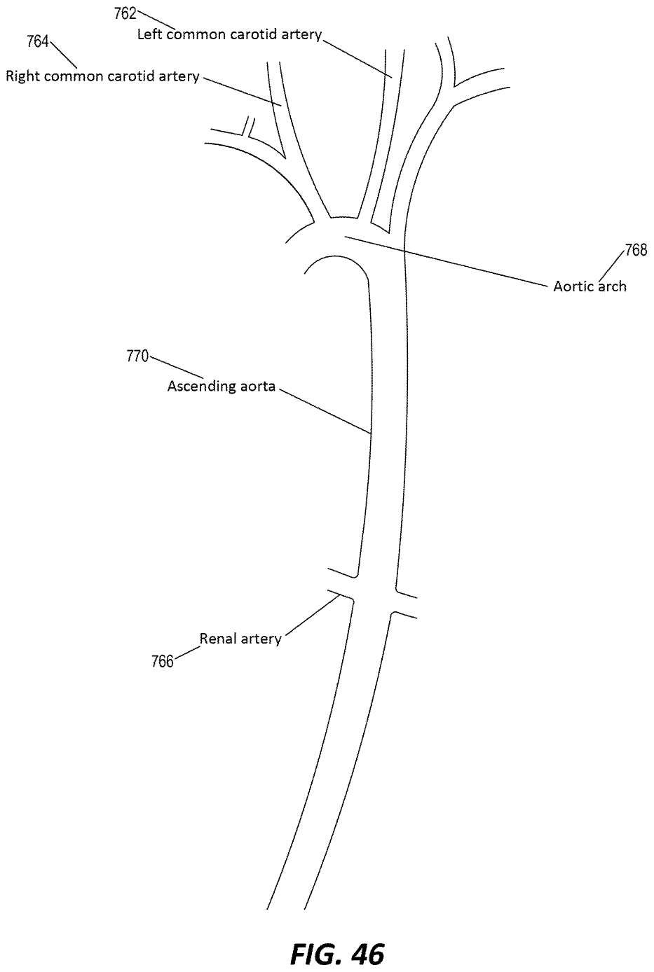

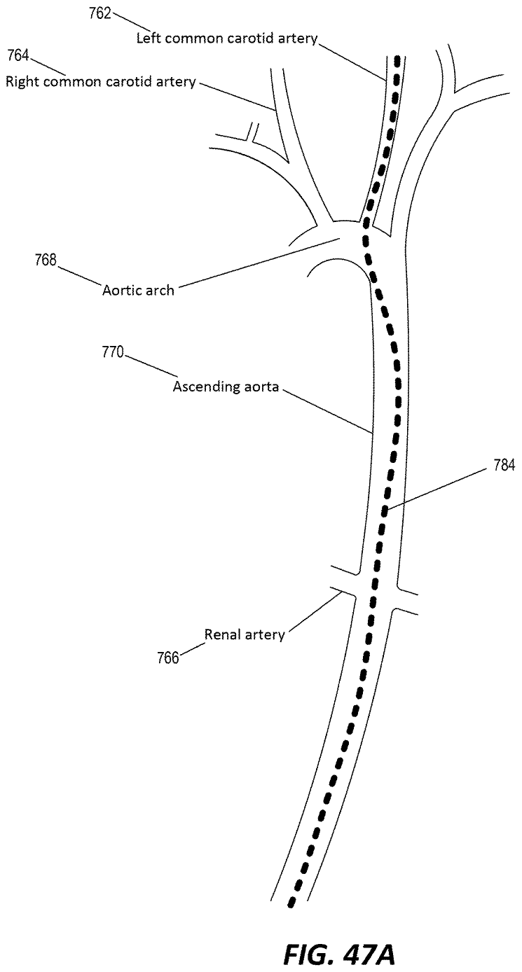

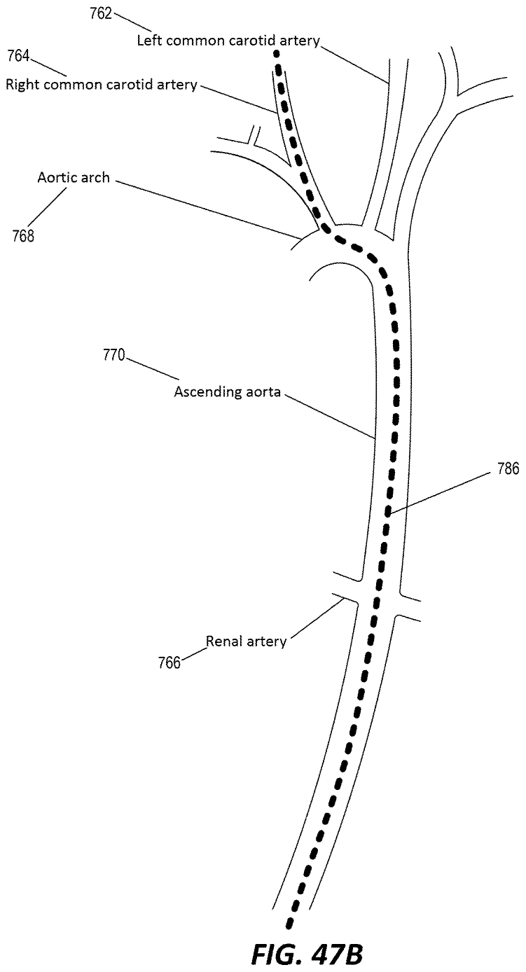

For example, referring to FIG. 46, a schematic diagram of the vascular anatomy between a femoral access point and portions of the central nervous system is illustrated, with two likely priority pathways for many given interventions: up through the right common carotid artery (764), or up through the left common carotid artery (762), each of which would generally require navigation from femoral access past the renal artery (766) branches, up the ascending aorta (770), into the aortic arch (768), and into the selected carotid branch. Referring to FIG. 47A, for example, such a pathway may be prescribed by an operator in a computing user interface configured to display portions of the patient actual anatomy or computerized representations or models thereof which are based upon preoperative and/or intraoperative data. For example, a pathway (784) shown through the anatomy in dashed line may be manually selected, or automatically selected with editing and/or approval manually, to result in a prescribed pathway (784) as shown through the left common carotid artery (762). FIG. 47B illustrates a similar configuration, but with a prescribed pathway (786) through the right common carotid artery (764). An associated computing system may be configured to bring to display image-related data or models pertaining to the actual patient anatomy, to intake from an operator various inputs or selections regarding initial access point (such as a selectable point on the femoral artery), ultimate distal destination (such as a location within the distal right common carotid artery), and other instructions or prescriptions such as keep out zones, structures to avoid, theoretical load ceilings, and other factors such as general collision avoidance; such information may be utilized by the associated computing system to prescribe a pathway, or at least an initial pathway which may be approved and/or manually adjusted by an operator before execution of movement under computerized control (i.e., such as by interconnected magnetic field application and vectoring, as described, for example, in reference to FIG. 20B).

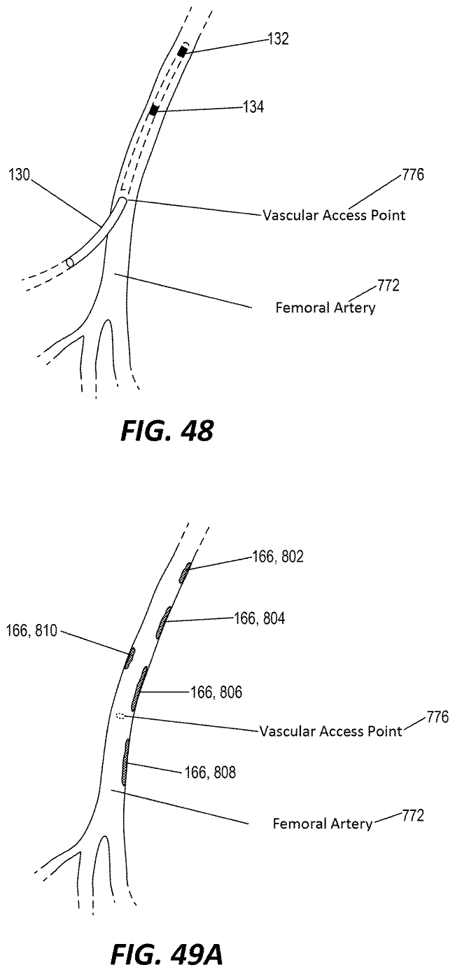

With a prescribed pathway selected, the system may be configured to guide the elongate instrument (130) along such pathway with closed-loop control based upon real-time or near-real-time imaging confirmation provided by systems such as ultrasound, fluoroscopy, localization, encoder reading, or others, as described, for example in reference to FIG. 20B. For example, referring to FIG. 48, an elongate instrument (130) is shown being navigated through a vascular access point (776) and up the femoral artery (772) in a collision avoidance type of navigation configuration wherein the elongate instrument (130) is shown travelling down the middle of the selected vascular pathway.

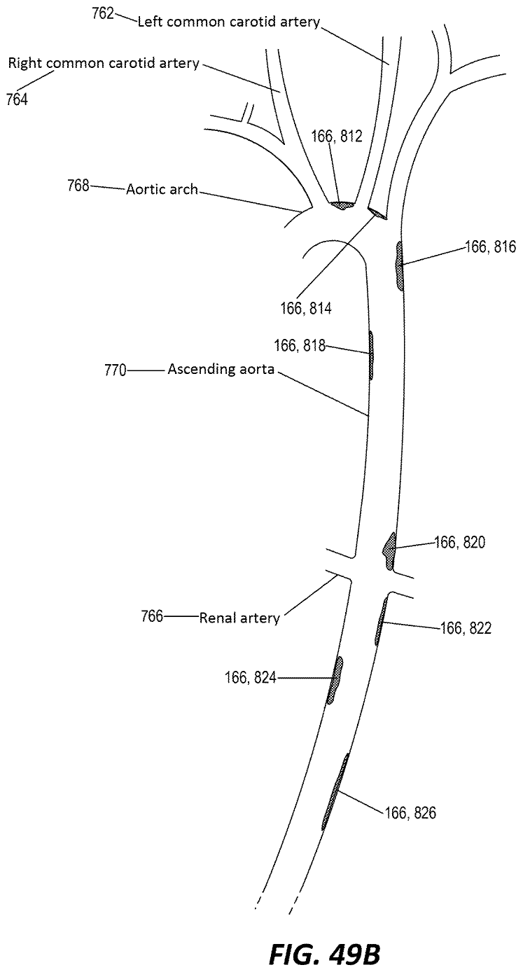

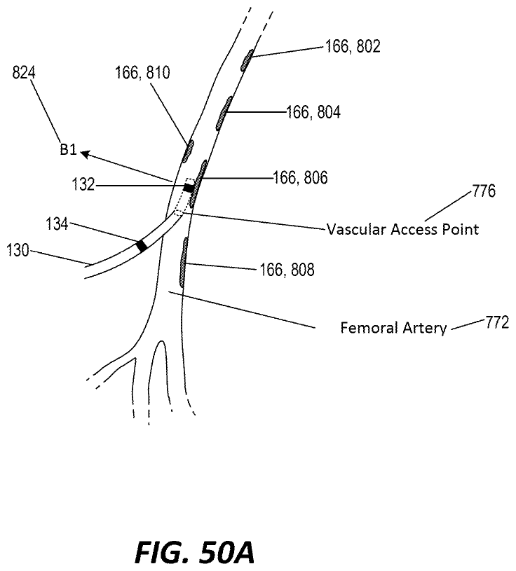

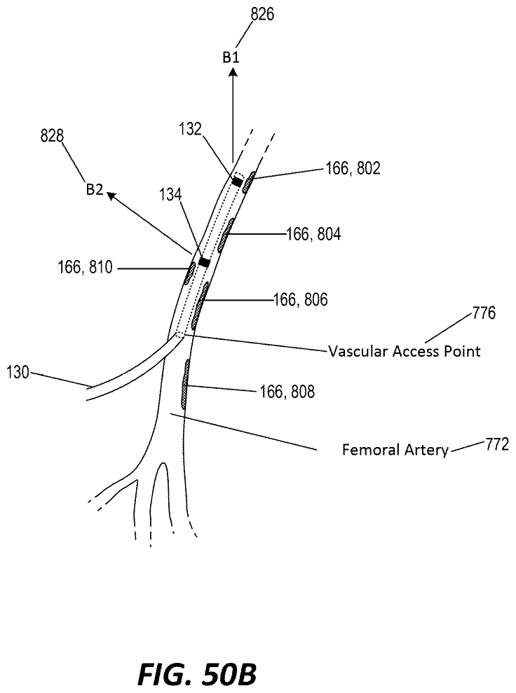

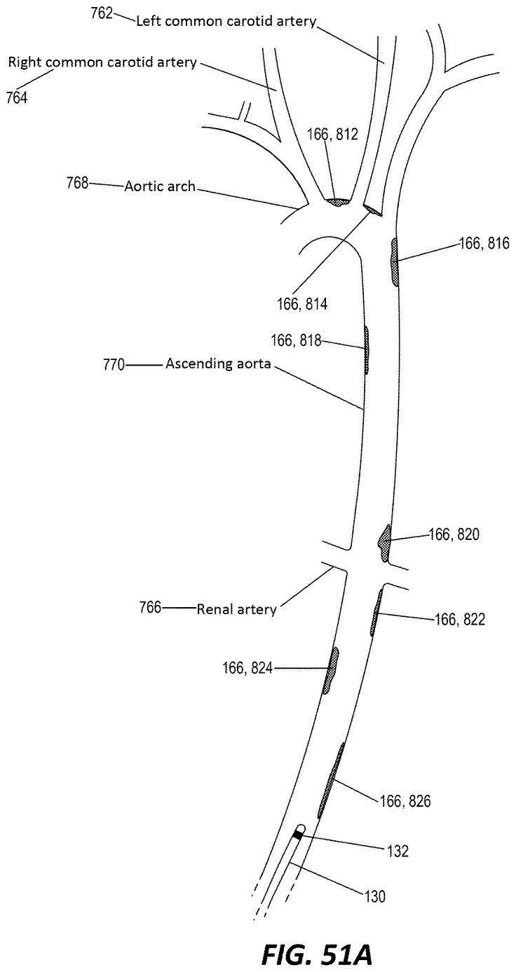

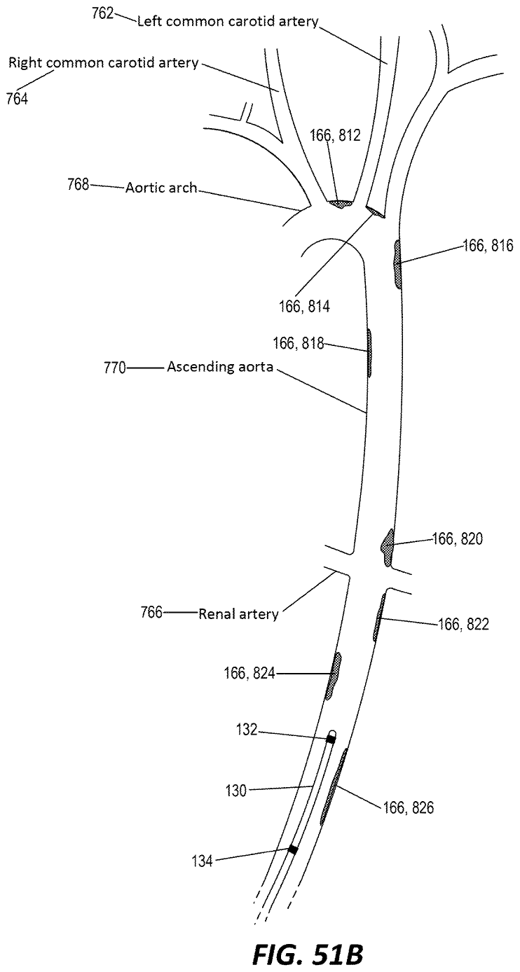

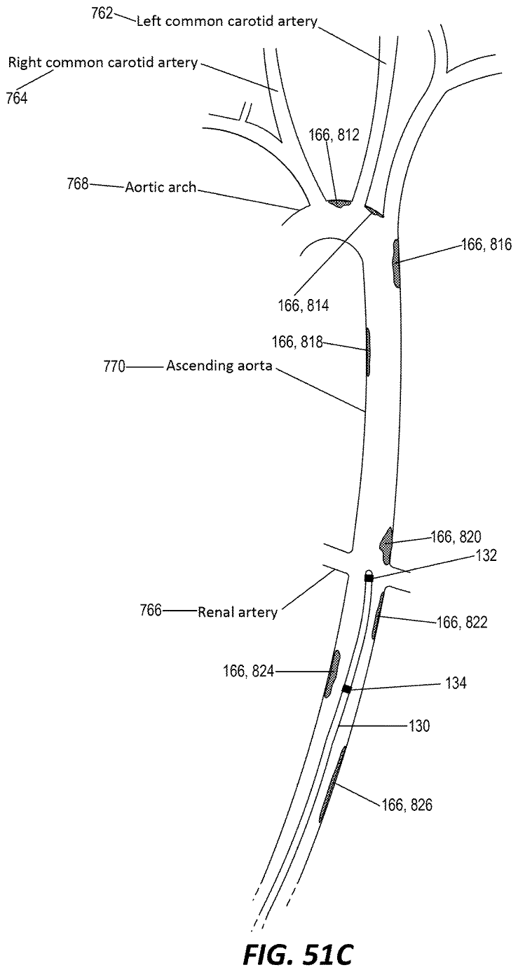

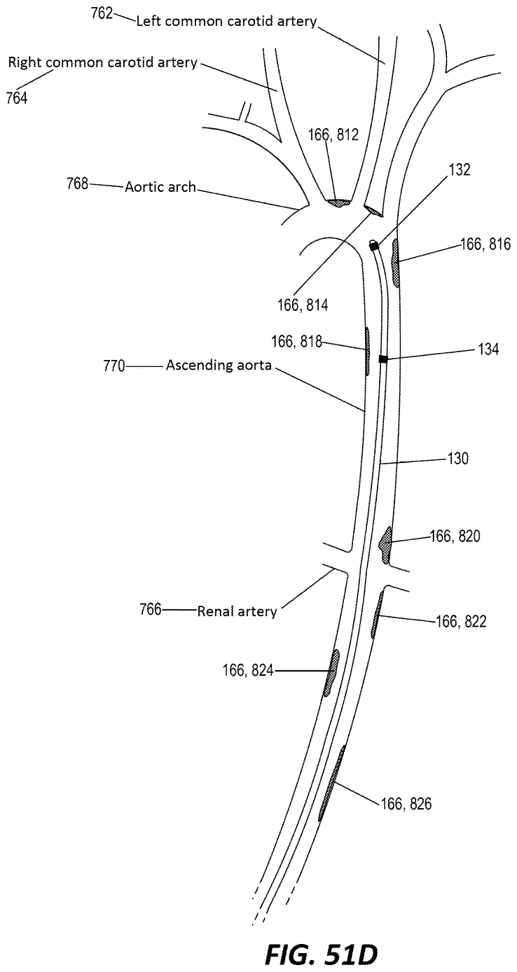

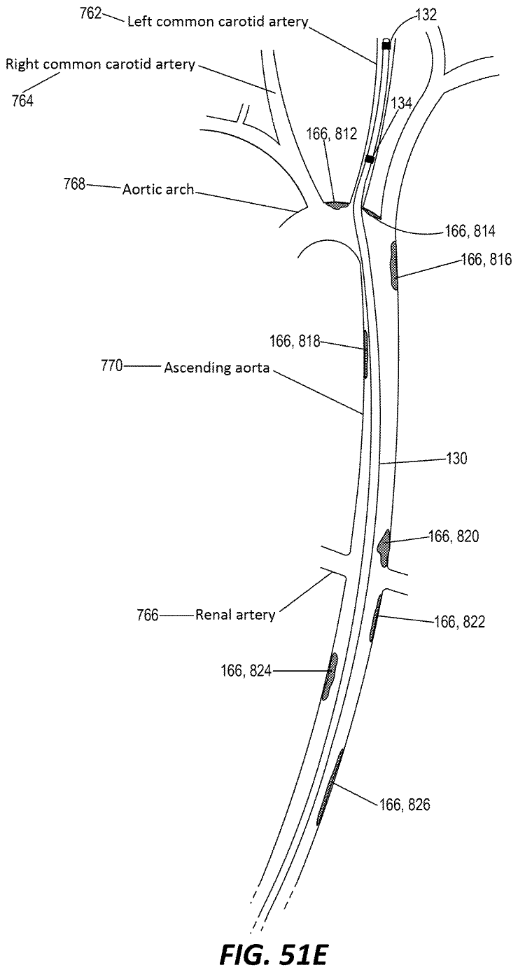

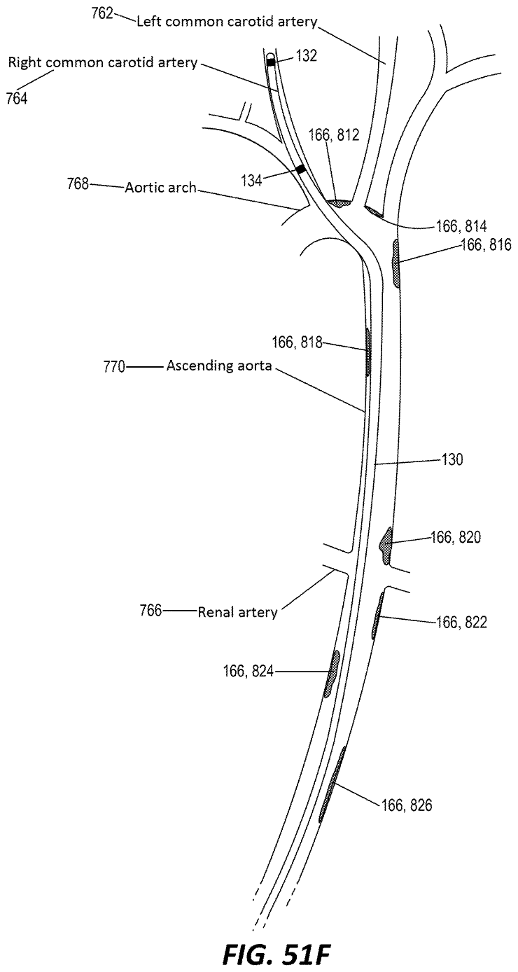

Referring to FIGS. 49A-49B, often the navigation is significantly more challenging, with prescribed keep away zones, regions, or objects, such as plaques, stents or stent grafts, possibly unstable vascular endothelial lesions, and other challenges. FIG. 49A illustrates a plurality of endoluminal objects (166) such as various plaques (802, 804, 806, 808, 810) which may be designated for avoidance, for example, in the proximity of the prescribed femoral access point (776). Similarly, FIG. 49B illustrates a plurality of endoluminal objects (166) such as various plaques (812, 814, 816, 818, 820, 822, 824, 826) which may be designated for avoidance, for example, in the more distal portions of the prescribed vascular pathway near the central nervous system/brain of the patient. FIGS. 50A-50B and 51A-51F illustrate advancement of the elongate instrument (130) as assisted by applied and vectored magnetic fields or magnetic flux, insertion/retraction control, and roll control, as described above in reference to FIG. 20B, for example, with the intercoupled computing system assisting to automatically avoid selected keep-away obstacles (such as 802, 804, 806, 808, 810 and/or 812, 814, 816, 818, 820, 822, 824, 826). It is noteworthy that as a magnetically controllable assembly such as that shown in FIGS. 50A-50B and 51A-51F is to be navigated relative to such depicted anatomy, there are many factors at issue in parallel as the system and operator best plot and navigate relative to the anatomy and hazards or risks associated thereto. For example, in various embodiments, it may be desirable to have magnetic steerability elements (132, 134) and magnetic field sources which are specifically configured to have little overlap for very specific navigability of an entire distal shape of the elongate instrument relative to the anatomy, for example. In other words, in certain variations, it may be desirable to have relatively discrete and independent control over each of the multiple nodes to produce complex shapes during instrument navigation, although if there is field overlap or coupling between nodes, the system (i.e., via the intercoupled computing resource) may be configured to handle this complexity while still accomplishing navigational complexity.

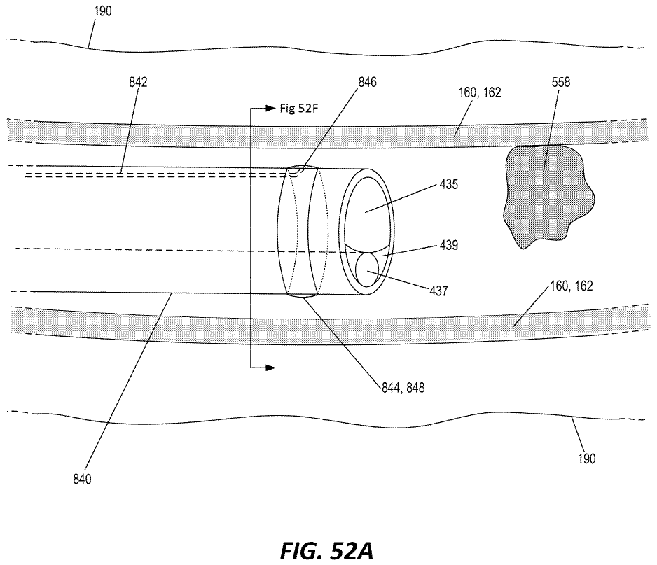

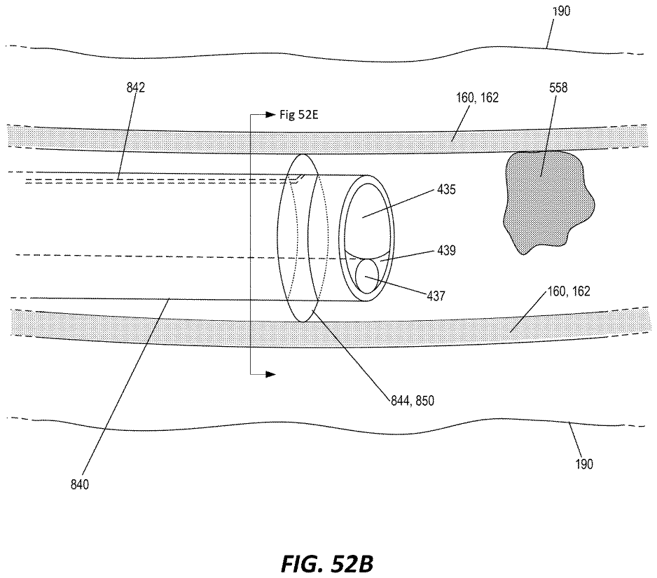

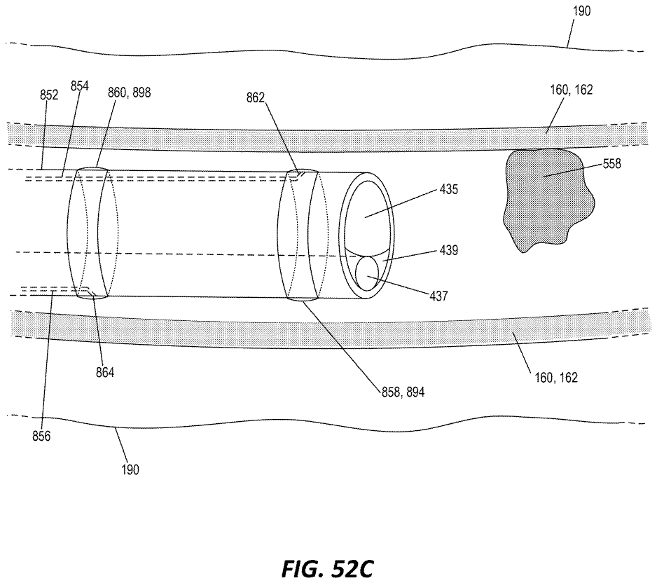

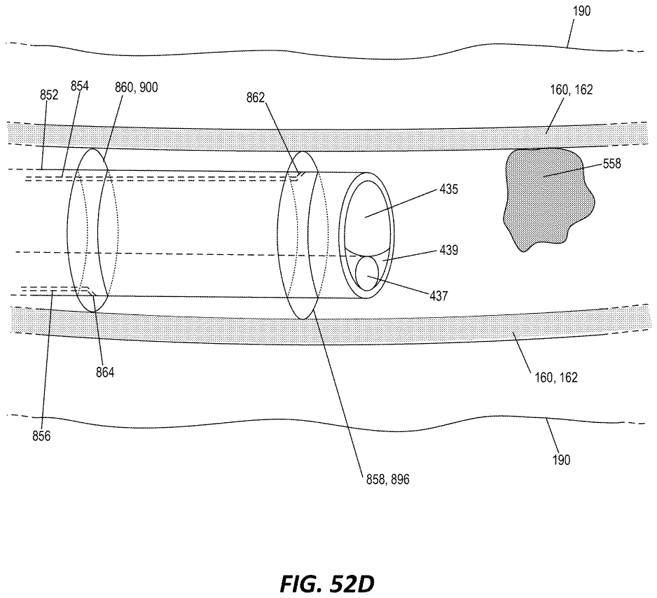

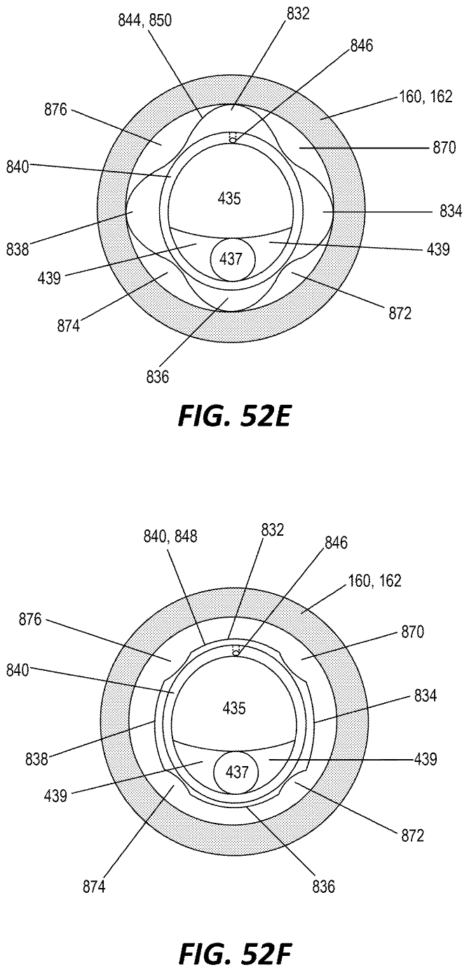

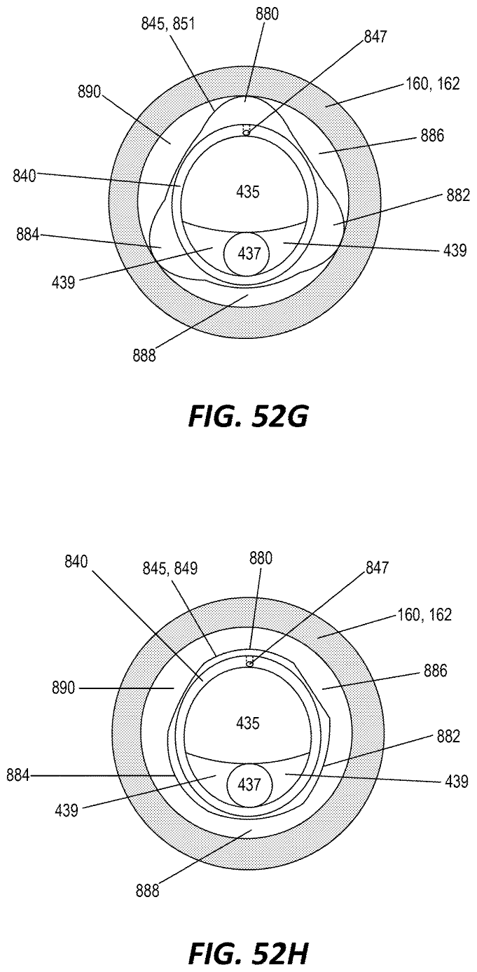

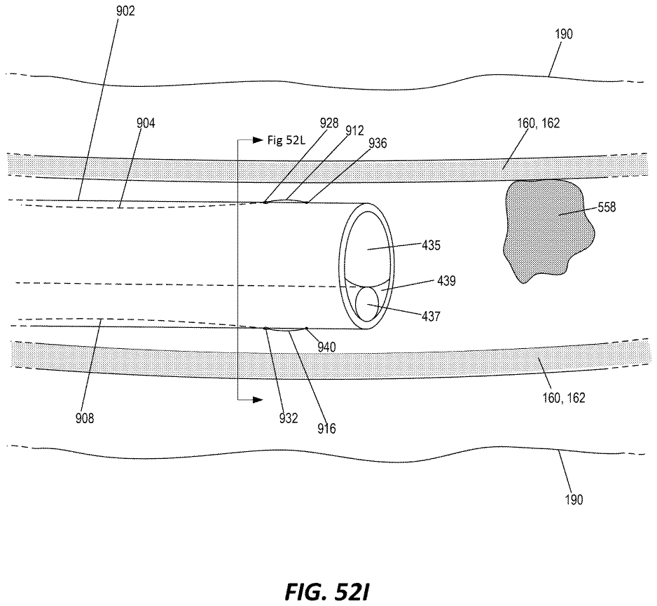

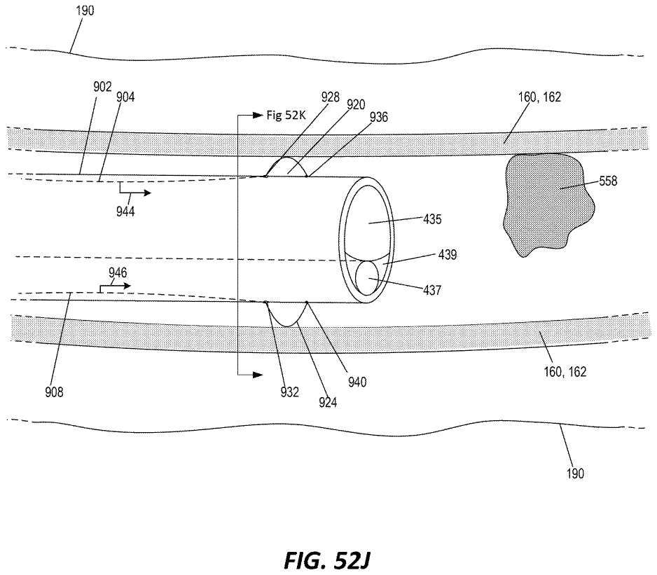

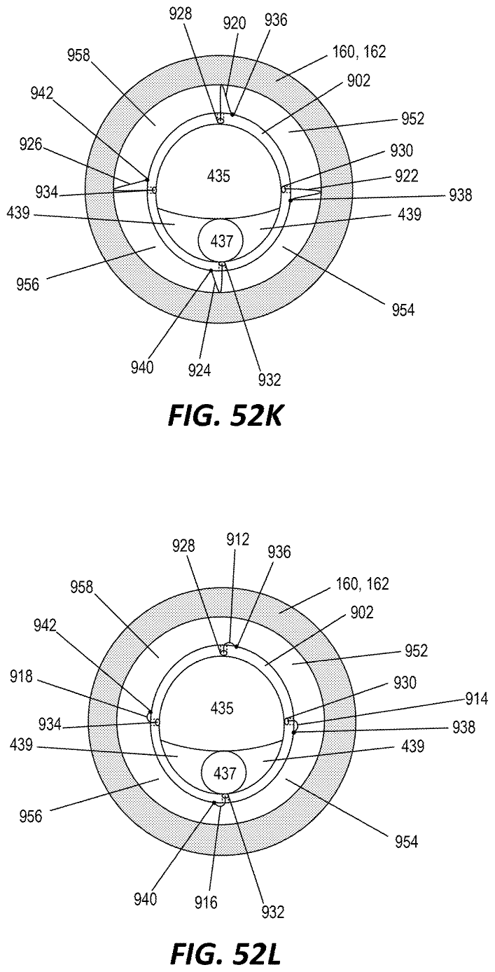

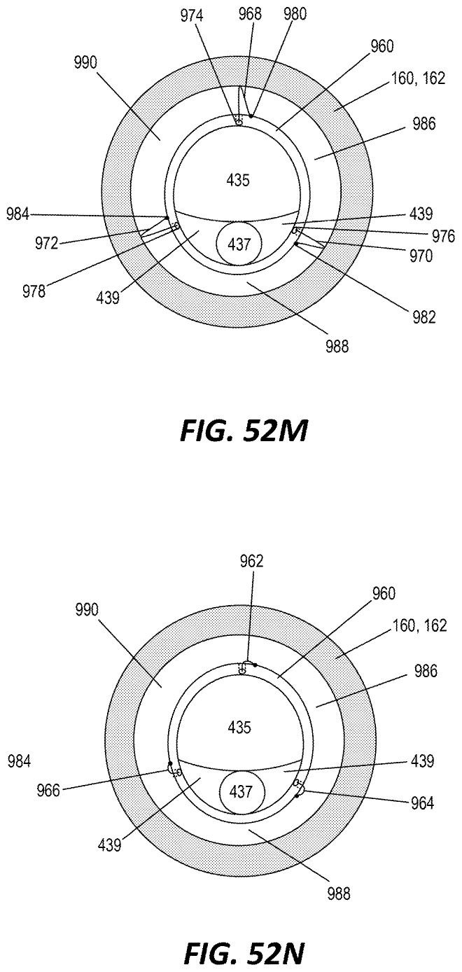

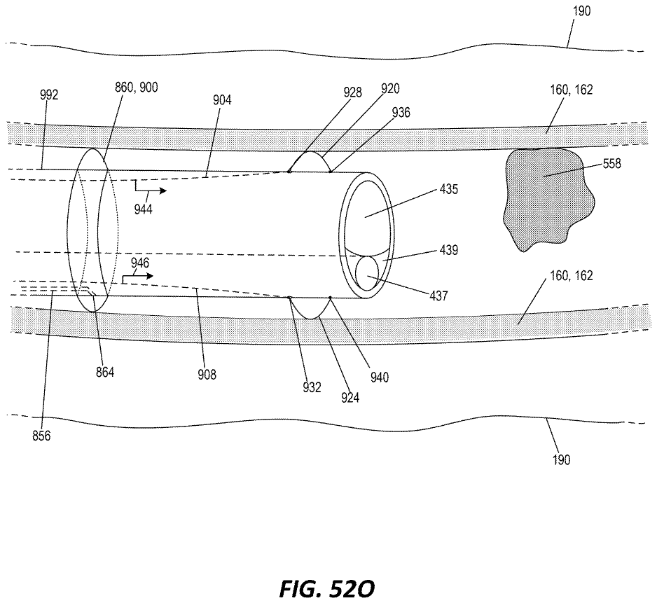

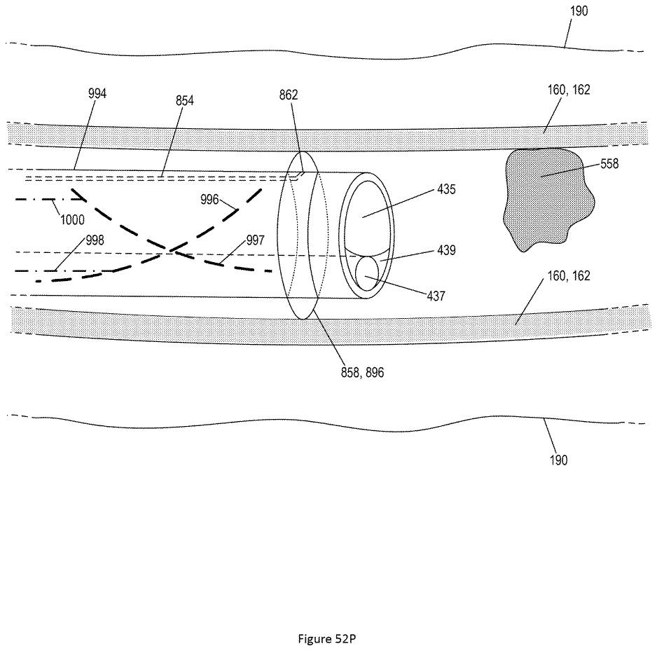

Referring to FIGS. 52A-52P, in various embodiments it may be desirable to have a physically-stabilizing capability for an elongate instrument (130) once it has navigated to a location within close proximity to targeted anatomy or a targeted lesion, clot, or other object. As shown in FIGS. 52A and 52B, an expandable stabilization feature (844) for a probe or instrument (840) such as a multi-lobed expandable balloon structure may be coupled to an elongate instrument (130) and fluidly coupled (such as via an inflation/deflation lumen 842 which joins 846 to inflate an inner chamber of the expandable stabilization feature 844) to a proximal inflation or deflation facility (such as a pump or small pressure-creating reservoir positioned extracorporeally and controllable by an operator). FIG. 52A and cross-sectional view FIG. 52F illustrate the expandable stabilization feature 844 in a collapsed state (848) more convenient for navigation and delivery; FIG. 52B and cross-sectional view FIG. 52E illustrate the expandable stabilization feature 844 in an expanded state (850) such that it is configured to engage the nearby tissue structure (such as a lumen 160 or vessel 162) while also providing a cross-sectional multi-lobed (832, 834, 836, 838) geometry to allow fluid such as blood to flow past through the non-occluded inter-lobe gaps (870, 872, 874, 876). FIGS. 52G and 52H illustrate embodiments parallel to those of FIGS. 52E and 52F to demonstrate that other pluralities of lobes for an expandable stabilization feature (845) having controllably collapsed (849) and expanded states (851) may be effective at facilitating stability while also allowing for bypass flow; the configuration of FIGS. 52G and 52H has three lobes (880, 882, 884) and three inter-lobe gaps (886, 888, 890). FIGS. 52C and 52D illustrate a probe or instrument (852) configuration wherein two successive expandable stabilization features (858, 860) may be utilized to provide further stability. Each may be configured to have a collapsed state (894, 898) and an expanded state (896, 900) and a plurality of lobes for bypass flow; each may have an inflation/deflation lumen (854, 856; each fluidly coupled 862, 864) to control/change state (expanded or collapsed).

Referring to FIGS. 52I and 52J, in other embodiments, one or more extendable stabilization features (904, 908), such as highly-flexible wires made from materials such as Nitinol (nickel-titanium alloy), may be inserted (944, 946) or retracted such that distal portions controllably expand out (i.e., expand out between exit apertures 928/932 and distal coupling points 936/940; collapsed configurations 912/916 are shown in FIG. 521; expanded configurations 920/924 are shown in FIG. 52J) to provide physical stabilization of the instrument or probe (902). FIGS. 52L and 52K provide cross-sectional views parallel to FIGS. 52I and 52J, respectively. The variations of FIGS. 52K and 52L illustrate four extendable stabilization features (extended: 920, 922, 924, 926; collapsed: 912, 914, 916, 918; apertures 928, 930, 932, 943; distal coupling points 936, 938, 940, 942) with open bypass flow gaps between (952, 954, 956, 958). FIGS. 52M and 52N show configurations parallel to those of FIGS. 52k and 52L to illustrate a configuration with three extendable stabilization features (extended: 968, 970, 972; collapsed: 962, 964, 966; apertures 974, 976, 978; distal coupling points 980, 982, 984) with open bypass flow gaps between (986, 988, 990).

FIG. 52O illustrates a configuration with one set of extendable stabilization features (904, 908; similar to that illustrated in FIG. 52J) and one set of expandable stabilization features (860; similar to that illustrated in FIG. 52J) to illustrate that hybrid or combined configurations may be utilized for dynamic stabilization of a probe or instrument (992).

Referring to FIG. 52P, a probe or instrument (994) configuration is illustrated having a distal expandable stabilization feature configuration (858; similar to that illustrated in FIG. 52D) as well as a plurality of internal wall dynamic stiffener members (996, 997; may comprise materials such as shape-memory alloy which may be braided or fused into the wall of the instrument or probe 994 and configured to become urged to change shape when provided with a controlled current stimulation, such as through a control lead (998, 1000, respectively) ). To assist in stabilizing a distal portion of an instrument (994) as a home platform for further intervention, it may be desirable to not only physically stabilize such instrument (994) relative to tissue structures or other structures surrounding it, as with expandable or extendable stabilization members, but also to controllably and dynamically stiffen at least a portion of the instrument (994) body.

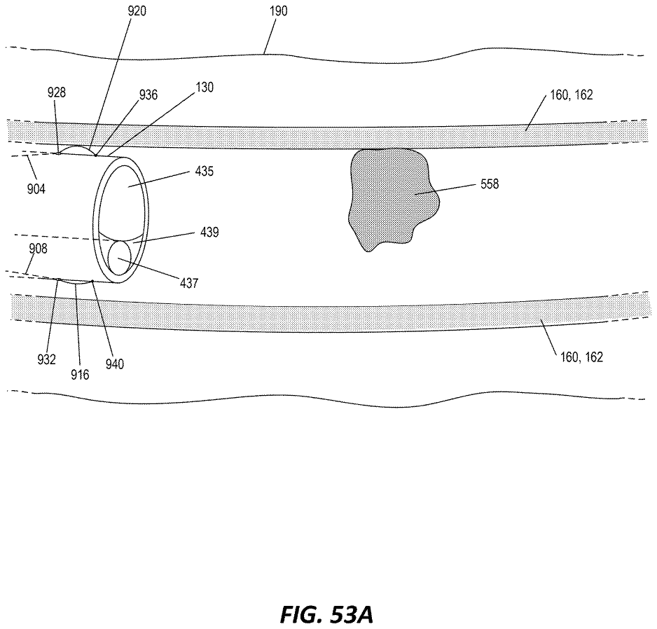

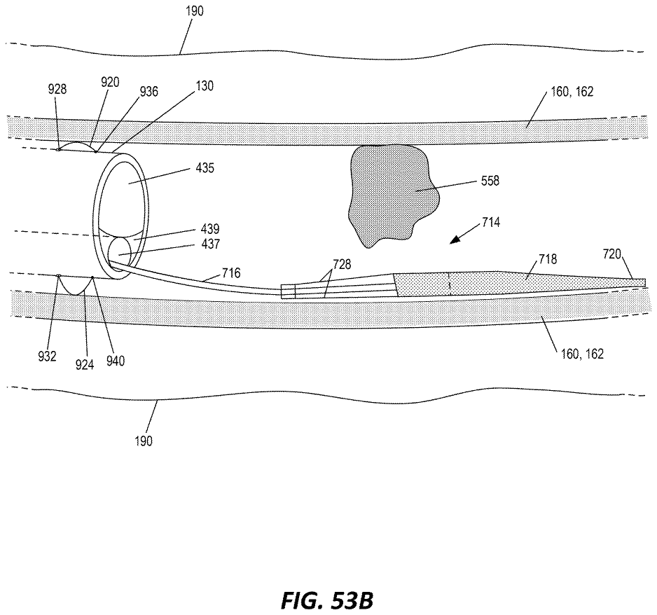

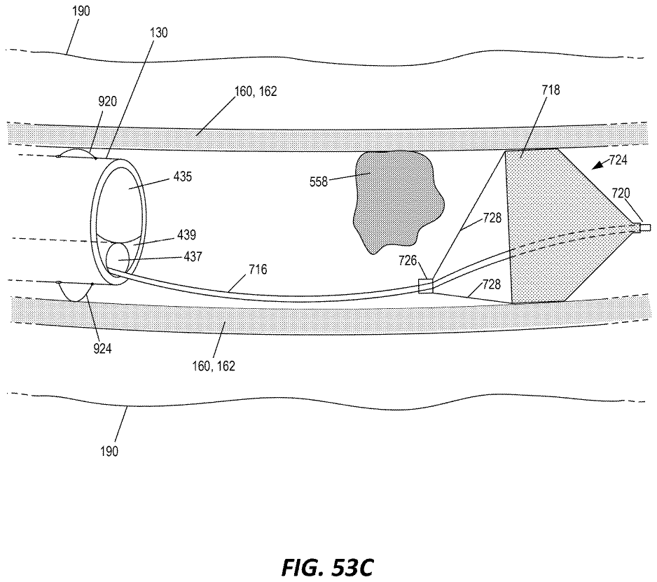

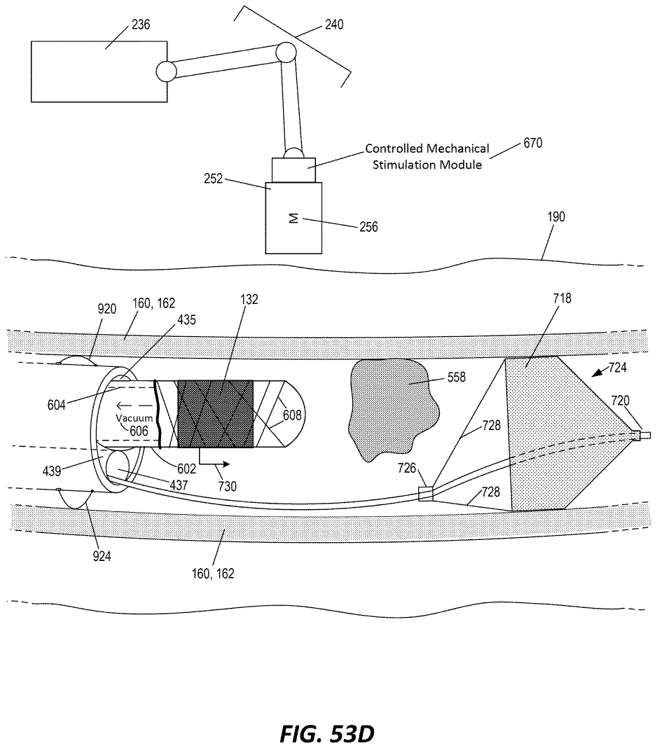

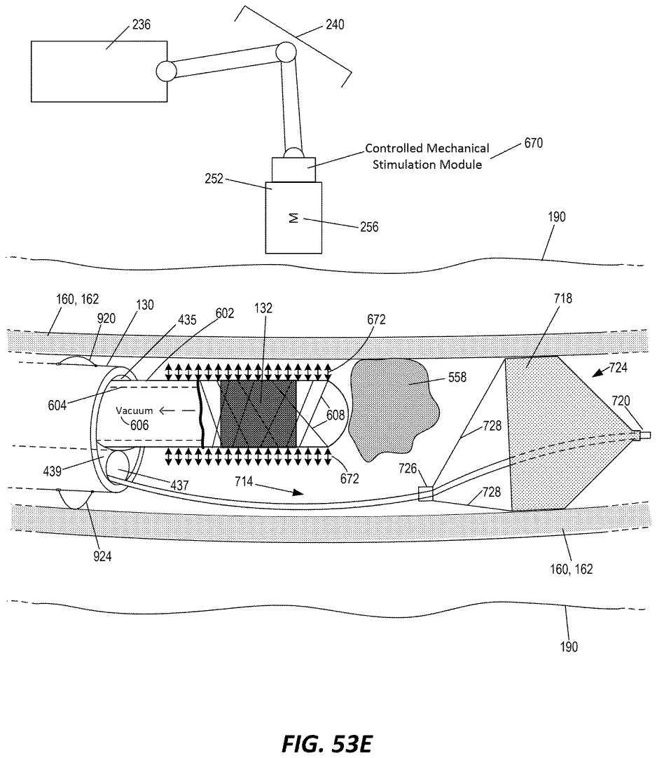

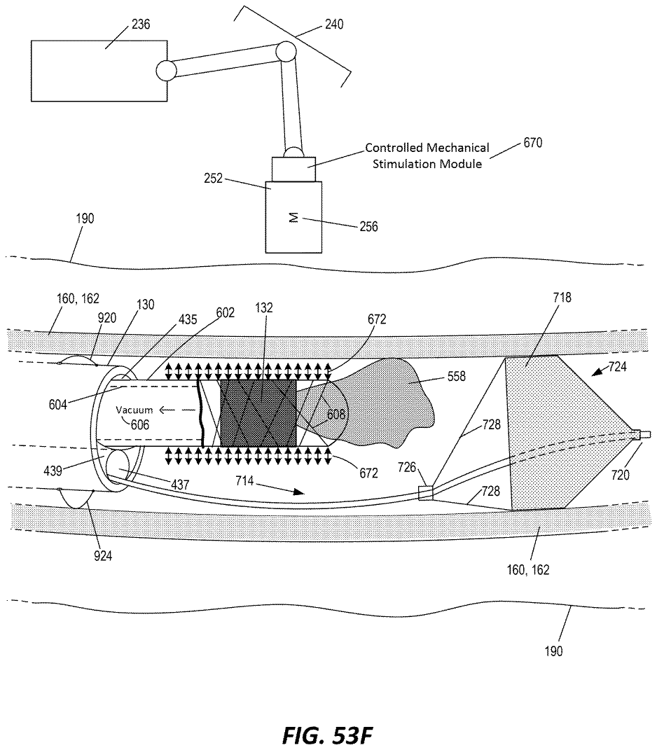

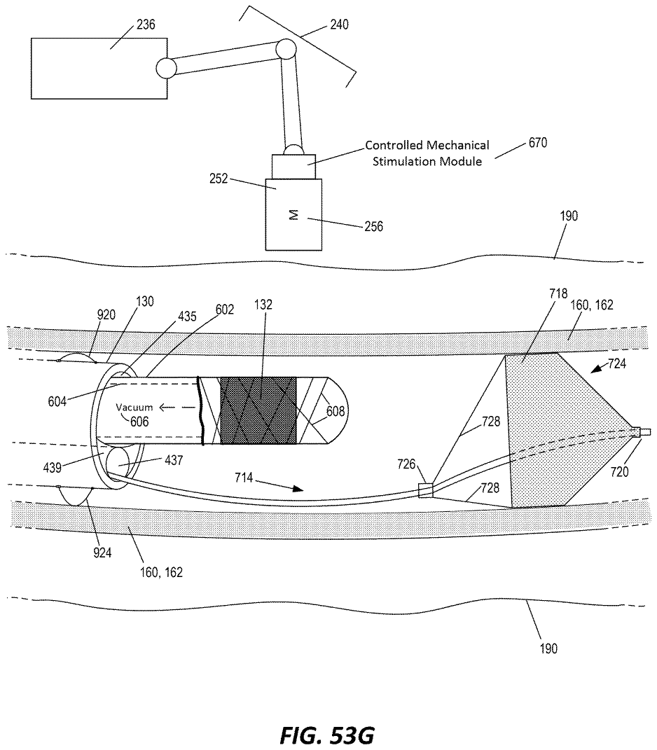

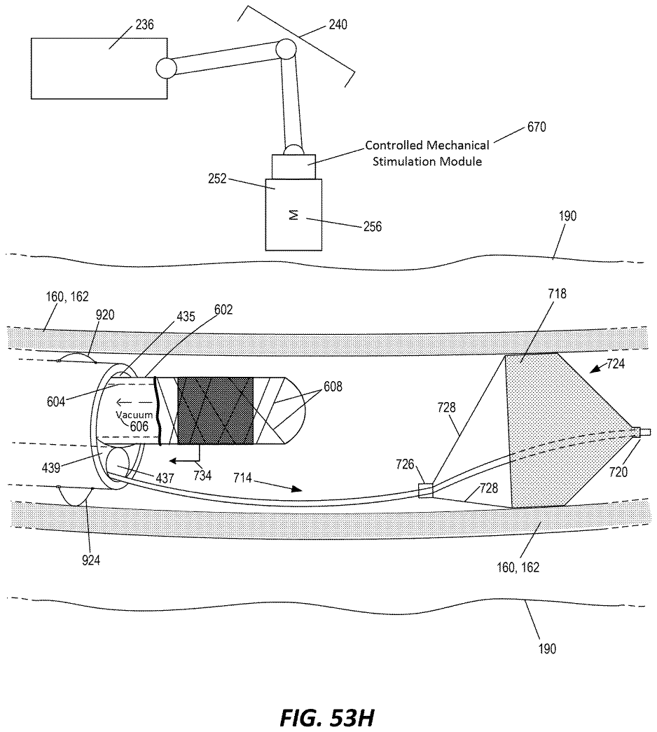

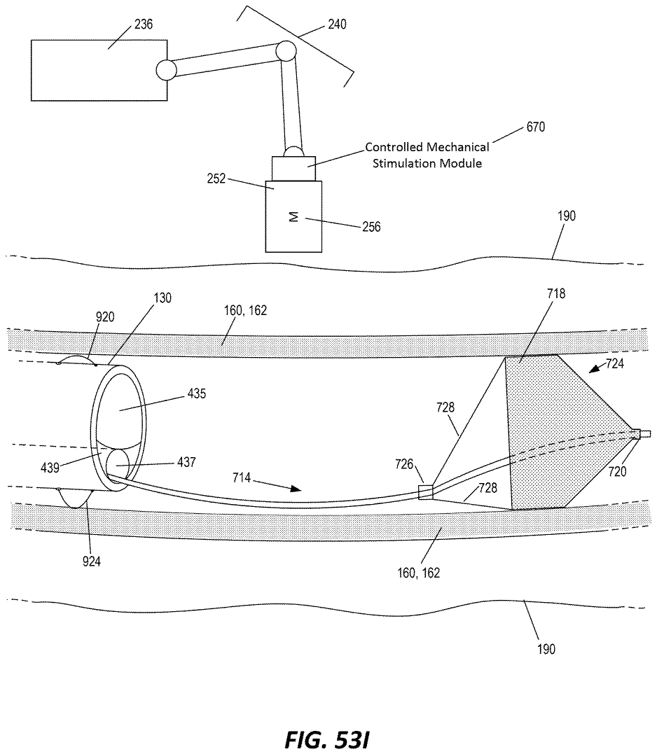

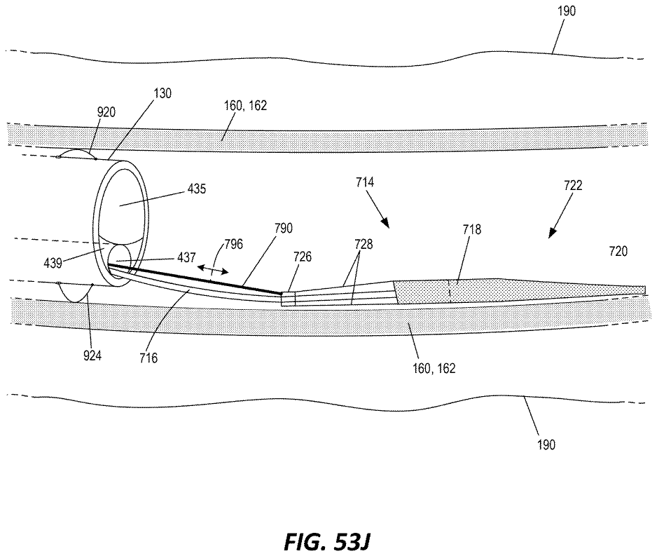

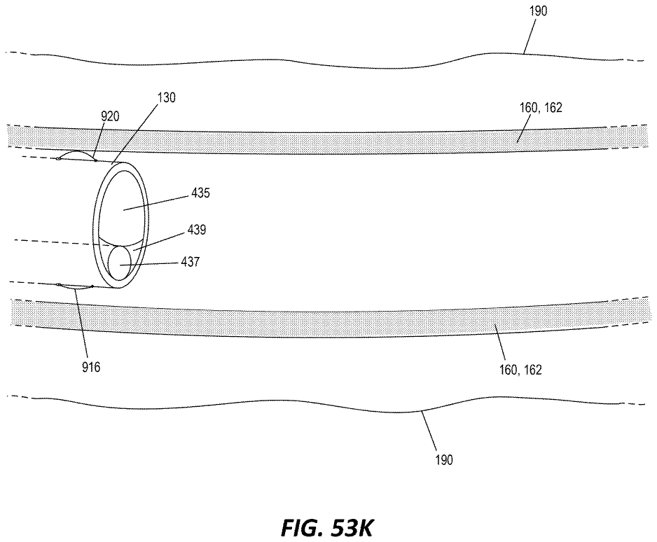

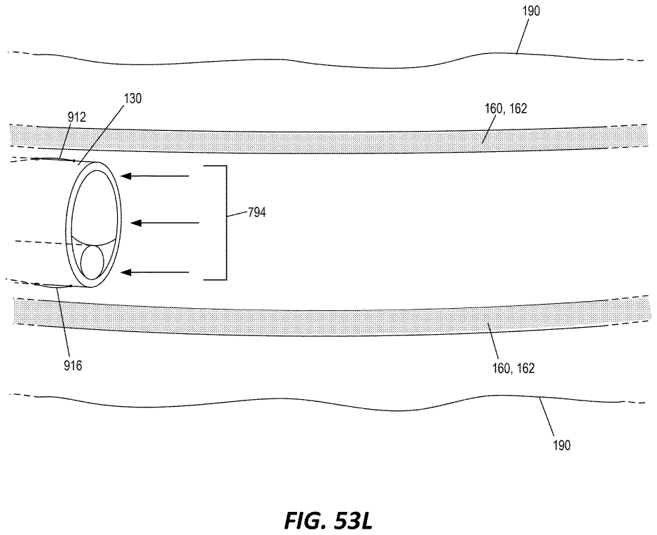

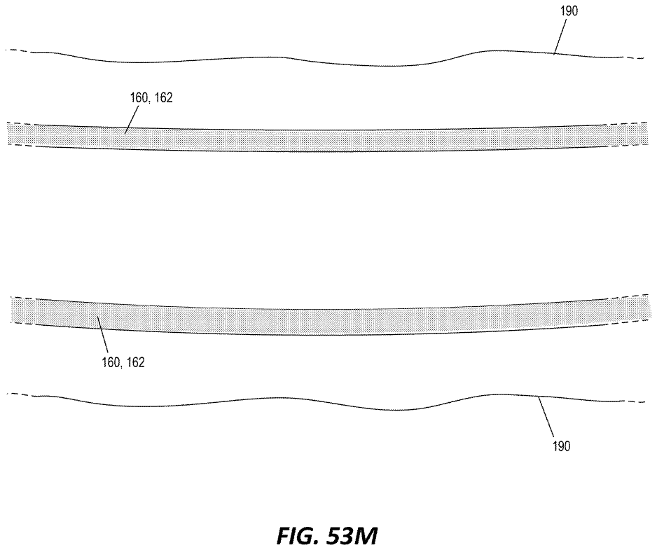

Referring to FIGS. 53A-53M, another illustrative clinical scenario is shown featuring various of the aforementioned configurations and variations. As shown in FIG. 53A, a magnetically steerable elongate instrument (130) is illustrated advanced through a lumen (160) such as a portion of the patient vascular (162) system into a position adjacent to a clot, thrombus, or embolism (558). In preparation for further activity from the “home platform” of the distal portion of the positioned elongate instrument (130), one or more stabilization structures, such as the extendable stabilization structures shown (one in expanded form 920, the other in collapsed form 916 in preparation for controlled conversion to expanded form for assisting with stability of the instrument 130; as described, such as in reference to FIGS. 52I and 52J, may be controllably expanded or collapsed using proximally extending portions 904/908 which may be pushed or pulled relative to the instrument 130) may be utilized to controllably stabilize the distal portion of the instrument (130). Referring to FIG. 53B, with the distal portion of the instrument (130) stabilized (920, 924), a distal protection assembly (714) may be deployed and advanced past the clot, thrombus, or embolism (558) in a collapsed form (722). Referring to FIG. 53C, the distal protection assembly (714) may be converted to expanded form (724; such as via utilization of a pull or push wire control lead, such as that 790 shown in FIG. 53J) to assist in preventing particles, portions, or structures from moving past the area of operation. Referring to FIG. 53D, a working instrument (602; similar to that described in reference to FIG. 35 above, for example) may be introduced and utilized to capture the clot (558) or portions thereof using vacuum (606), or vacuum combined with mechanical disruption and/or dislodging assistance from a probe distal portion having a loose mesh (608) type of construction configured to capture and/or disrupt clot/embolic/thrombus material. The probe 602 may be advanced 730 toward and into physical engagement with the clot 558 for such activity, as shown in FIG. 53E. Also shown in FIG. 53E, in addition to controlled insert/retraction, roll, and general steering such as +/−pitch and +/−yaw (such as provided by use of configurations as described, for example, in reference to FIG. 20B), controlled oscillatory motion (672) may be induced (256, 670) and utilized to assist in disruption and/or removal of the clot (558). Referring to FIGS. 53F and 53G, with the clot loosened and/or disrupted, it may be removed using the vacuum (606) capability of the probe (130) as shown, and referring to FIGS. 53H and 531, the probe (602) may be controllably retracted (734) away into the elongate instrument (130). Referring to FIG. 53J, the control lead (790) may be utilized to pull or push (796; here pull to collapse) the proximal coupler to induce the collapsed (722) state of the distal protection assembly (714) so that it may be retracted into one of the working lumens (437) of the elongate instrument (130) as shown in FIG. 53K. Also shown in FIG. 53K, with the portions of the clot removal operation completed, the stability configuration may be reverted to a collapsed configuration for retraction of the elongate instrument itself (130); FIG. 53K illustrates one of the extendable stabilizer members already in collapsed configuration (916), with the other still in expanded configuration (920) just before it is returned to a collapsed configuration for instrument (130) removal. FIG. 53L illustrates proximal retraction (794) of the elongate instrument (130) from the operational area with collapsed (912, 916) extendable stabilizer members; FIG. 53M illustrates the operational area with all instrumentation removed.

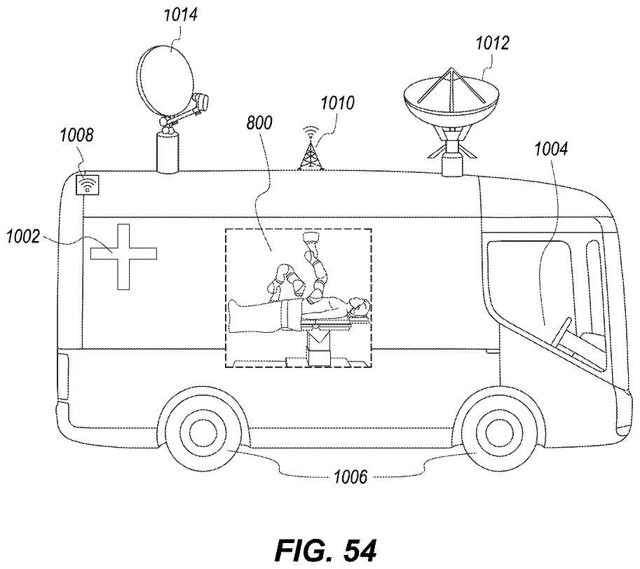

Referring to FIG. 54, to decrease time to intervention when such time can be absolutely critical (such as in the case of certain ischemic stroke scenarios), it may be desirable to have certain aspects of systems such as that described above in reference to FIG. 16 (800) available on a mobile or potentially-mobile basis. Such a system may be coupled to a stable base of a relatively large vehicle (1002) which may have a stabilized suspension, a compartment for medical operators/drivers, and sophisticated connectivity to facilitate computing and communications integration with experts at other locations. For example, in the depicted variation, various high-speed connectivity alternatives such as microwave transmission (1014), mobile cell connectivity (1010), IEEE 802.11 (1008), and/or satellite communications (1012) may be utilized to not only process various aspects of the procedure, such as magnetic steerability vectoring, but also high fidelity video presence and communication for teleoperation and remote presence (for example, a top neurosurgeon may be able to teleoperate or become virtually present in the vehicle through the telecommunications capability, and may be granted access to patient information such as background files, medical records, and imaging data which may be captured at the patient/vehicle location, for example).

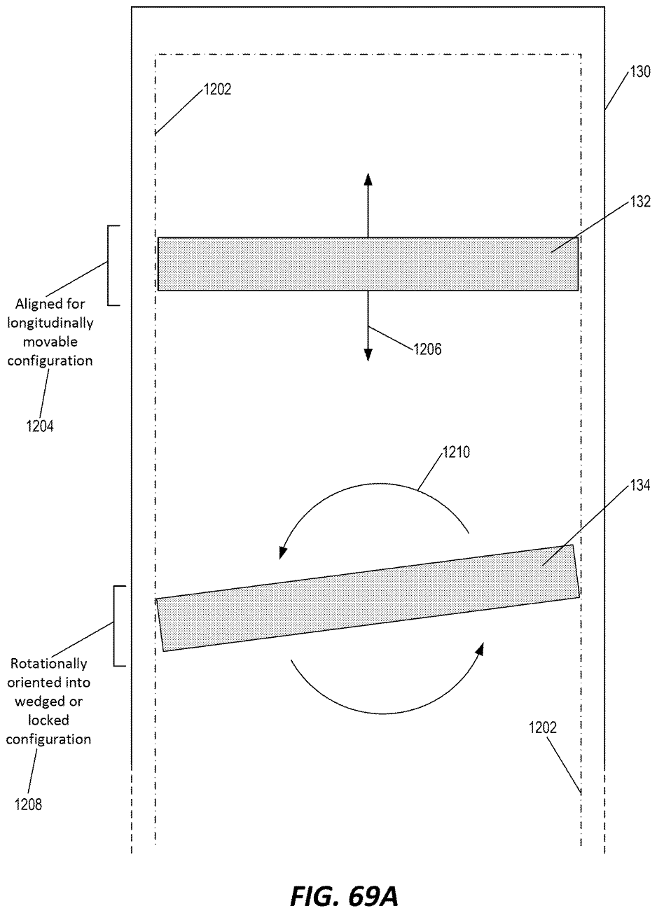

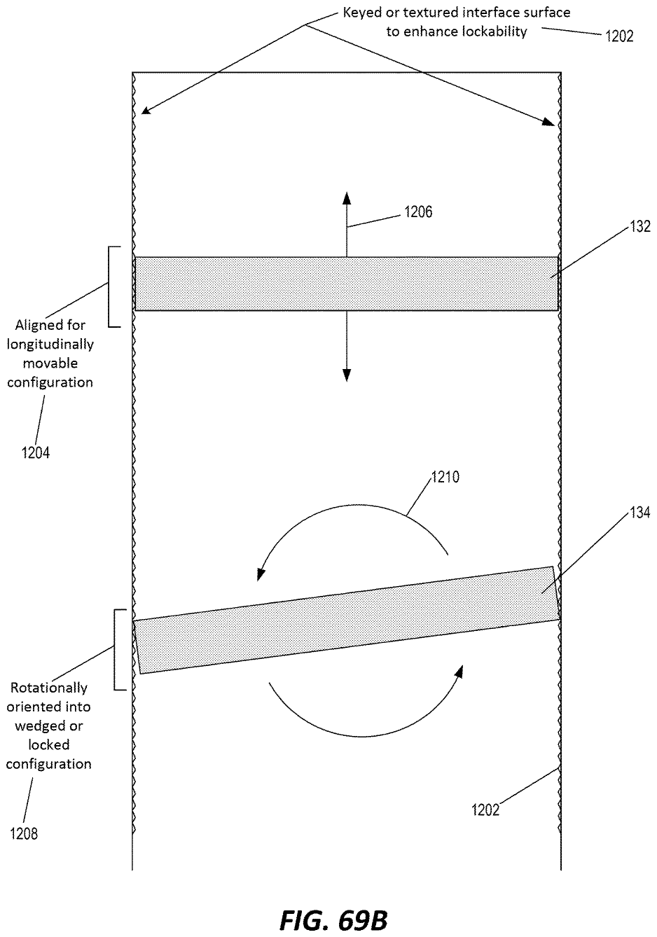

Many of the above described configurations involve the integration of various computer-controlled or implemented technologies, such as robotic arms, to position and orient magnetic elements and/or sensing devices or transducers, address potential controlled adjustment of positioning of magnetic elements along the length of an instrument (see, for example, discussion below pertaining to FIGS. 69A and 69B), conduct aspects of image processing, image registration, optional pathway or navigation estimation and determination, closed-loop control pertaining to prescribed operator objectives (such as keep-away zones), and generally planning and executing in reference to operational objectives which may be prescribed by one or more operators. For example, a seemingly simple exercise of navigating an elongate instrument 130 from one location to another may entail: co-register fluoroscopy, ultrasound, plane radiography, and MRI data from preoperative imaging; co-register preoperative image information to intraoperative image information, on a real-time or near-real-time basis, subject to operator confirmation before use in navigation execution; utilize available sensors, such as localization and image information registration, to present and confirm co-registration and available closed-loop control; navigate from femoral access point to identified targeted tissue structure location within the neurovasculature using co-registered image information from preoperative and intraoperative sources as well as available localization, subject to manual interrupts and only at limited joint velocities with limited geometric envelopes per operator prescription/settings; generally avoid contact where possible; do not contact prescribed keep-away zones without manual override from operator during navigation; present stabilization control panel when at prescribed destination for execution of mechanical stabilization such as via inflation or other expandable or extendable members). Indeed, various additional control and operational issues may be brought simultaneously to issue with various procedures and portions thereof. For example, referring back to FIGS. 50A-50B, 51A-51F, 52A-52P, and 53A-53M, it is important to note that the subject system is configured to provide for control of not only a single control element or degree of freedom, but a plurality of control elements and/or degrees of freedom simultaneously—to provide for control (position, rotation, insertion, orientation, loading, oscillatory motion mode, and/or vacuum or jet mode, for example) pertaining to an elongate portion of an instrument body, for example. In other words, rather than control of a discrete tip or other discrete portion, the subject system is configured to provide for simultaneous and independent control of a plurality of portions or regions of the associated instrument, as described above and illustrated, for example, in FIGS. 13A-13I. Thus with appropriate operatively coupled computing resource, such instruments may be simultaneously operated for multi-nodal geometric and/or loading control (such as with a plurality of magnetic steering elements, such as 132, 134) using multiple precision-guided magnetic fields (guided, such as via closed-loop robotics as described above), with other multiple-mode control features (such as oscillatory motion or vibratory motion mode [such as a preferred clot agitation oscillatory motion configuration as opposed to a smooth instrument motion/relatively-low-friction low-amplitude oscillatory motion configuration], focused/applied energy control, instrument insertion and/or or roll control and/or load application, distal protection deployment and utilization, distal stabilization deployment and utilization, magnetic steering node repositioning, vacuum and/or or fluid insertion control, grasping or other control structure control, and/or other control degrees of freedom, for example, such as those described in reference to FIGS. 19A-19E, 20A-20B, 21A-21C, 24, 25, 26A-26F, 27, 28A-28D, 29A-29E, 30A-30D, 31, 32A-32C, 33A-33C, 34, 35, 36, 37A-37B, 38A-38B, 39A-39H, 52A-52P, 53A-53M, 68A-68C, and/or 69A-69B.

To assist with parallel-processing such issues, computing configurations featuring neural networks, such as different neural networks, may be utilized, such that applied statistics and known patterns from training (such as from training via supervised, semi-supervised, and/or self-training learning configurations using data from the same or similar system scenarios) may be utilized to assist in controls paradigms. Simulation configurations may also be created to simulate the physical environments and physical behaviors and interactions pertaining to operation of instruments (such as an elongate instrument 130 with two magnetic steerability elements 132/134 at given distance apart, with various magnetic field strengths/vectors/positions) in various tissue structure environments (such as within a relatively large lumen such as the trachea, esophagus, or colon; such as within a smaller lumen such as the neurovasculature, small intestine, or a fallopian tube, for example; or such as within more free space, such as within a surgically insufflated portion of the abdomen or thorax of a patient). Further, in operation or at runtime, reinforcement learning models and multi-agent (or so-called “agentic” or “multi-agentic”) reinforcement learning models informed by operator reward settings/prescriptions (such as avoiding keep-away zones, reaching a targeted anatomical location goal, avoiding certain collisions, staying within certain boundaries, staying below certain joint velocities or instrument velocities, etc) may be utilized to assist in the parallel process of many commands and safety measures. In various embodiments, in the training of the reinforcement learning models, different reward and priority configurations may be selected and the model can be trained preoperatively for each key parameter. During the pre-operative consult or analysis phases, the surgeon or operator may, for example, select one of these pre-trained modalities which he or she believes will best fit the specific patient scenario. An example of this would be one of many possible such parameters such as minimally-invasive interaction, engagement, and/or avoidance of endoluminal plaque; another example of a selectable parameter would be most direct path to clot or thrombus. Indeed, with significant information and capability pertaining to simulation (i.e., a computerized synthetic catheter model moving within a model of human anatomy steerably operating subject to modelled magnetic fields vectored and applied using modelled robotic arms, for example), reinforcement learning models may be run/operated at relatively high frequency so that the system may be configured to learn and optimize operation, have more alternatives for dealing with more different scenarios or singularities, etc. Further, neural network models may be informed by actual data pertaining to actual cases and/or procedures of the past, as well as data pertaining to the subject/current case (i.e., the anatomy, patient at particular issue in pre-operative, intraoperative, and post-operative care), of course, such that the system may be continually advanced and/or improved. In various embodiments, these computing challenges may be handled by one or more local and/or remote computing resources. Further, multi-agent reinforcement learning configurations may be utilized to separate certain computing challenges. For example, multi-agent configurations with portions of the model known as “liquid neural network” reinforcement models may be utilized, wherein a desirable amount of flexibility is presented pertaining to allocation of computing and processing resources along with operational priorities and reinforcement model reward paradigms (which, again, may form a spectrum from something such as: never go beyond this prescribed geometric envelope that has been operator confirmed in the co-registered image fusion model of the patient anatomy; to something as precise as: do not let the instrument touch this object [say, for example, a previously placed cardiovascular stent] without manual override). Thus in one embodiment, a multi-agent reinforcement learning model with a liquid neural network may comprise many agents working for a specific outcome, but focused only on that outcome. Such liquid neural network may be utilized as one tool toward defining a global reward outcome to optimize an algorithmic outcome. One benefit of using liquid neural network configurations is that the weighting may be adapted based upon noise and shifting statistical information in the runtime of a procedure. A key aspect is the synthetic or so-called “simulation-to-real” model used for training these systems, as these may use a plurality of components such as sensor models, human anatomy, movement profiles, and many more factors in order to most accurately recreate a real scenario. With such multi-agent configurations, specific agents may be configured and prioritized to focus and functionally specialize. For example, as compared with a single-agent configuration assigned to address a multifactorial issue wherein the agent may have many tools at its functional disposal, many decisions about which tool or aspect to call next in operation, and a relatively large context to keep track of with a single agent, a multi-agent configuration may allow for a more modular approach wherein separate agents may be specifically developed, tested, maintained, and improved with greater specialization to address particular issues or domains, and generally enhanced control such that a supervising or mixing layer may be configured to explicitly address how agents communication and function relative to each other. For example, a cardiovascular intervention multi-agent configuration may comprise four or more cooperative agents: one solving for safety, one solving for contact loads at a specific location X (for example, to keep contact loads below a certain threshold at location X), one solving for contact loads at a specific location Y (for example, to completely avoid contact loads at location Y), and one solving for adherence to a predetermined or pre-selected instrument pathway relative to the tissue structures. Each agent may be relatively simple or relatively complex (for example, one or more agents may comprise one or more sub-agents or sub-sub-agents in a layer or hierarchy type of configuration), and may follow a conventional pattern or be customized to the tasks at hand (for example, the multi-agent configuration may comprise an architecture of agents particular to the domain of cardiovascular stroke intervention and may comprise shared keys for various agents addressing interoperability (or input/output, integration, or “I/O”) of the particular agents relative to each other. As noted above, an operational or control paradigm, or “mixing layer”, may be configured to operate agents relative to each other with various priorities and rewards within the multi-agent configuration, such as with various weightings applied, and a paradigm for addressing conflicts (for example, in various embodiments, a mixing layer may be configured to address competitive or adversarial reward functions with a general approach biased toward cooperation and modularity, and to utilize and further develop a meta data network which may be utilized to observe and predict outcomes in various actual or simulated configurations, somewhat in a manner akin to the operation of a modern self-driving road vehicle paradigm as such vehicle navigates roadways, hazards, and other dynamic issues; in various embodiments, the operational control or mixing layer may be configured to stop movement of the medical instrument relative to the subject patient tissue whenever there is uncertainty past a certain predetermined threshold, and to seek further input, for example, from an operator or aspects of the operational neural network and integrated resources). Indeed, also in a manner somewhat akin to the operation of a modern self-driving road vehicle, in various embodiments not all analysis at runtime is conducted dynamically and in real-time; the system may be configured such that it is trained and prepared to be brought into the medical procedure with an advanced background, as noted above, pertaining to the particular patient, the subject instruments, the intended medical procedure, information from previous procedures and simulation, pre-selected aspects of planning, pathways, and other factors (such as keep-away structures within the patient's cardiovascular system, load limits relative to aspects of the patient's cardiovascular system, targeted structures, weighting, priority, and/or reward functions or selections made by doctors or other operators, selected means of communicating with the system, such as via natural language, keyboard, physical master input device, and other factors); such background and preparation may be made available at runtime so that optimized alternatives are presented and selected without complete de-novo analysis of all factors in real-time. In other words, at runtime, certain dynamic factors may be presented in real-time that need to be optimally addressed, and while some aspects may be determined, calculated, correlated, or addressed using computing and/or operator resources in real-time or near-real-time, other factors may be presented with relative low-latency into the dynamic scenario because they are pulled and/or assembled from pre-existing information, calculations, correlations, or other information (i.e., not every single aspect needs to be calculated de-novo in real-time for every scenario; the system may be configured to take maximum advantage of training, simulation, and generally other integrated resources for the neural network operation). Further it is notable that certain agents or combinations thereof may be utilized in more than one process configuration, or reused many times and/or in multiple variations, within the same operation, procedure, or step (for example, a safety or non-collision agent may be ubiquitously utilized for various surgical procedures).

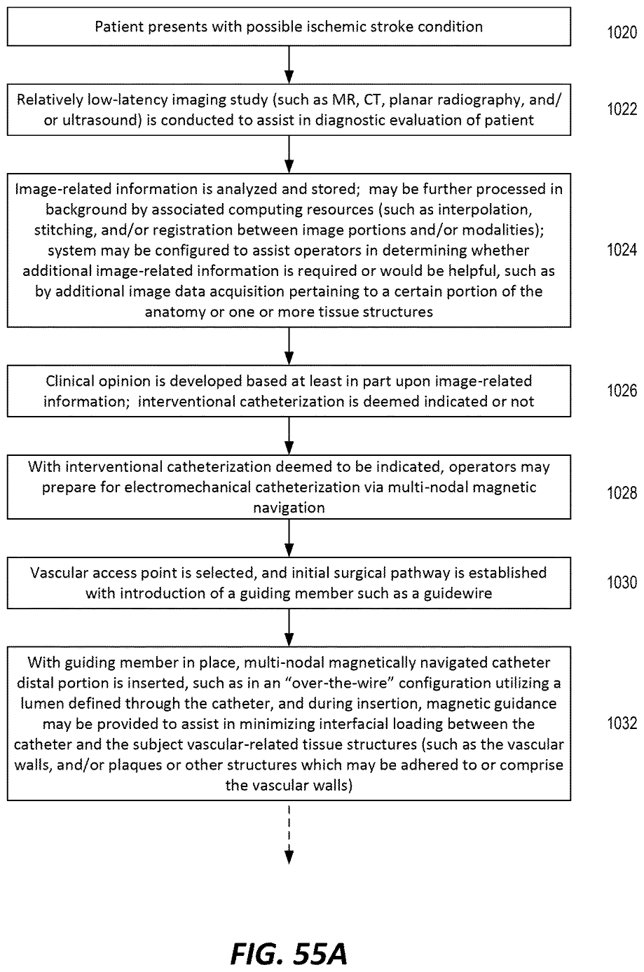

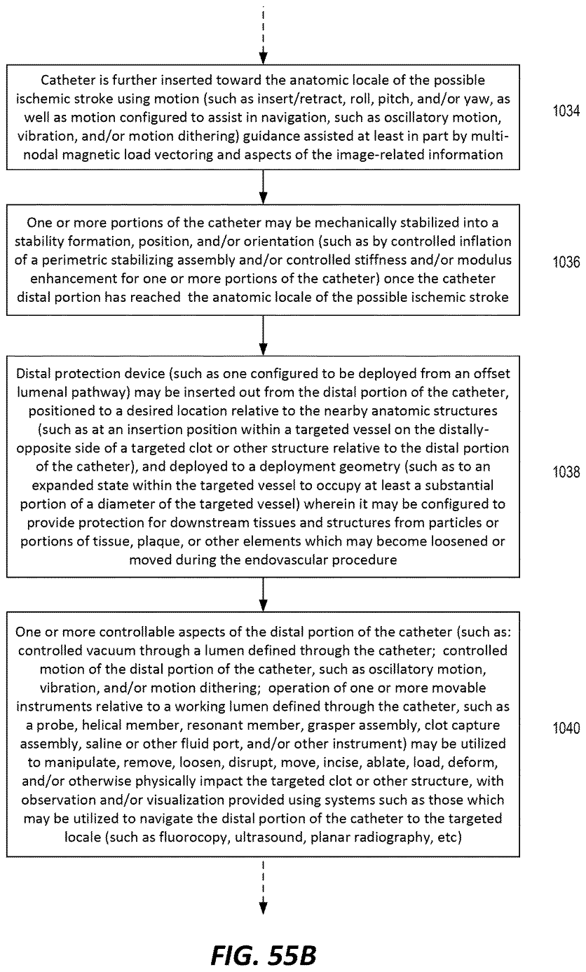

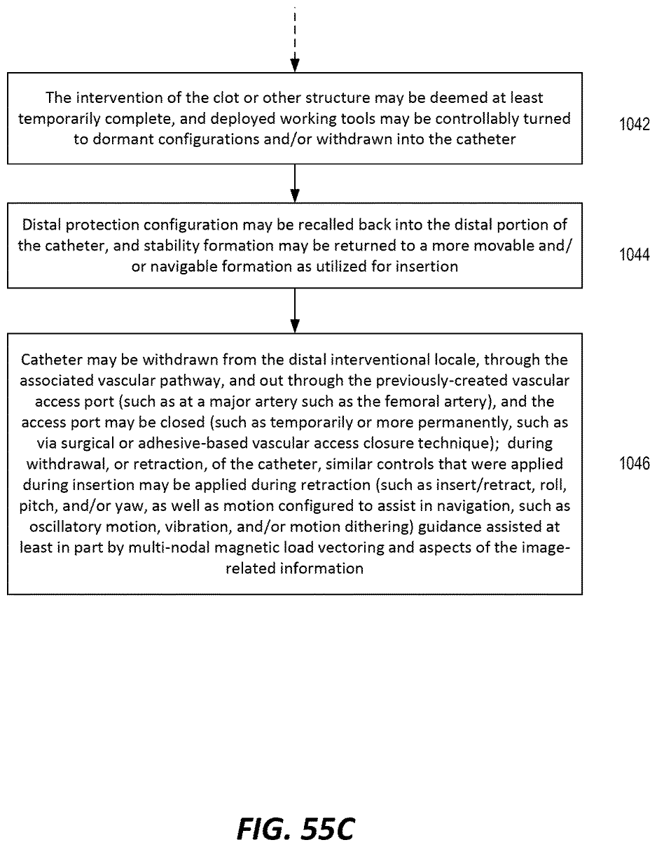

Referring to FIG. 55A, a patient may present with a possible ischemic stroke condition (1020). A relatively low-latency imaging study (such as MR, CT, planar radiography, and/or ultrasound) may be conducted to assist in diagnostic evaluation of patient (1022). Image-related information may be analyzed and stored; may be further processed in background by associated computing resources (such as interpolation, stitching, and/or registration between image portions and/or modalities); system may be configured to assist operators in determining whether additional image-related information is required or would be helpful, such as by additional image data acquisition pertaining to a certain portion of the anatomy or one or more tissue structures (1024). Clinical opinion may be developed based at least in part upon image-related information; interventional catheterization may be deemed indicated or not (1026). With interventional catheterization deemed to be indicated, operators may prepare for electromechanical catheterization via multi-nodal magnetic navigation (1028). A vascular access point may be selected, and an initial surgical pathway may be established with introduction of a guiding member such as a guidewire (1030). With guiding member in place, multi-nodal magnetically navigated catheter distal portion may be inserted, such as in an “over-the-wire” configuration utilizing a lumen defined through the catheter, and during insertion, magnetic guidance may be provided to assist in minimizing interfacial loading between the catheter and the subject vascular-related tissue structures (such as the vascular walls, and/or plaques or other structures which may be adhered to or comprise the vascular walls) (1032).

Referring to FIG. 55B and continuing from FIG. 55A, the elongate instrument or catheter may be further inserted toward the anatomic locale of the possible ischemic stroke using motion (such as insert/retract, roll, pitch, and/or yaw, as well as motion configured to assist in navigation, such as oscillatory motion, vibration, and/or motion dithering) guidance assisted at least in part by multi-nodal magnetic load vectoring and aspects of the image-related information (1034). One or more portions of the catheter may be mechanically stabilized into a stability formation, position, and/or orientation (such as by controlled inflation of a perimetric stabilizing assembly and/or controlled stiffness and/or modulus enhancement for one or more portions of the catheter) once the catheter distal portion has reached the anatomic locale of the possible ischemic stroke (1036). A distal protection assembly or device (such as one configured to be deployed from an offset lumenal pathway) may be inserted out from the distal portion of the catheter, positioned to a desired location relative to the nearby anatomic structures (such as at an insertion position within a targeted vessel on the distally-opposite side of a targeted clot or other structure relative to the distal portion of the catheter), and deployed to a deployment geometry (such as to an expanded state within the targeted vessel to occupy at least a substantial portion of a diameter of the targeted vessel) wherein it may be configured to provide protection for downstream tissues and structures from particles or portions of tissue, plaque, or other elements which may become loosened or moved during the endovascular procedure (1038). One or more controllable aspects of the distal portion of the catheter (such as: controlled vacuum through a lumen defined through the catheter; controlled motion of the distal portion of the catheter, such as oscillatory motion, vibration, and/or motion dithering; operation of one or more movable instruments relative to a working lumen defined through the catheter, such as a probe, helical member, resonant member, grasper assembly, clot capture assembly, saline or other fluid port, and/or other instrument) may be utilized to manipulate, remove, loosen, disrupt, move, incise, ablate, load, deform, and/or otherwise physically impact the targeted clot or other structure, with observation and/or visualization provided using systems such as those which may be utilized to navigate the distal portion of the catheter to the targeted locale (such as fluorocopy, ultrasound, planar radiography, etc) (1040).

Referring to FIG. 55C and continuing from FIG. 55B, the intervention of the clot or other structure may be deemed at least temporarily complete, and deployed working tools may be controllably turned to dormant configurations and/or withdrawn into the catheter (1042). The distal protection configuration may be recalled back into the distal portion of the catheter, and stability formation may be returned to a more movable and/or navigable formation as utilized for insertion (1044). The elongate instrument or catheter may be withdrawn from the distal interventional locale, through the associated vascular pathway, and out through the previously-created vascular access port (such as at a major artery such as the femoral artery), and the access port may be closed (such as temporarily or more permanently, such as via surgical or adhesive-based vascular access closure technique); during withdrawal, or retraction, of the catheter, similar controls that were applied during insertion may be applied during retraction (such as insert/retract, roll, pitch, and/or yaw, as well as motion configured to assist in navigation, such as oscillatory motion, vibration, and/or motion dithering) guidance assisted at least in part by multi-nodal magnetic load vectoring and aspects of the image-related information (1046).

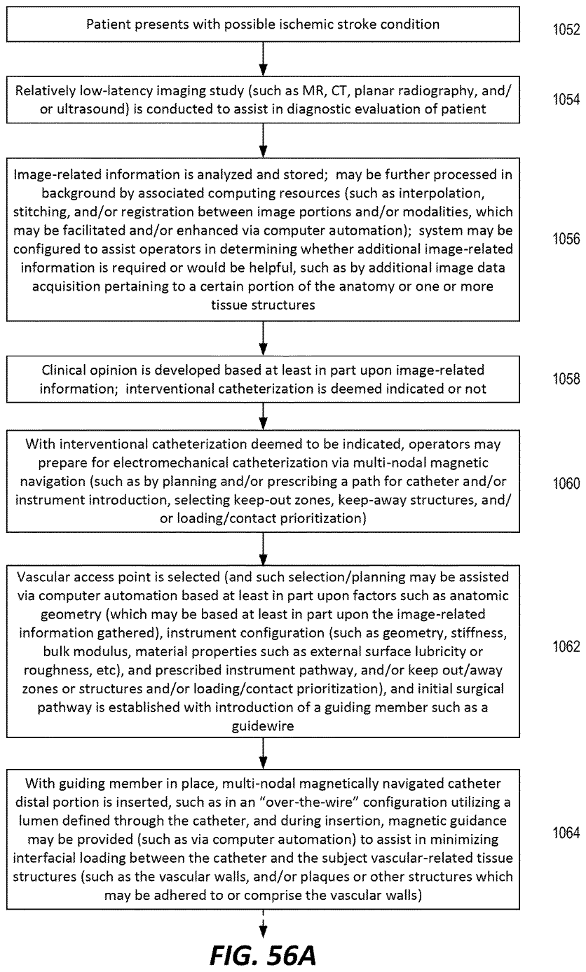

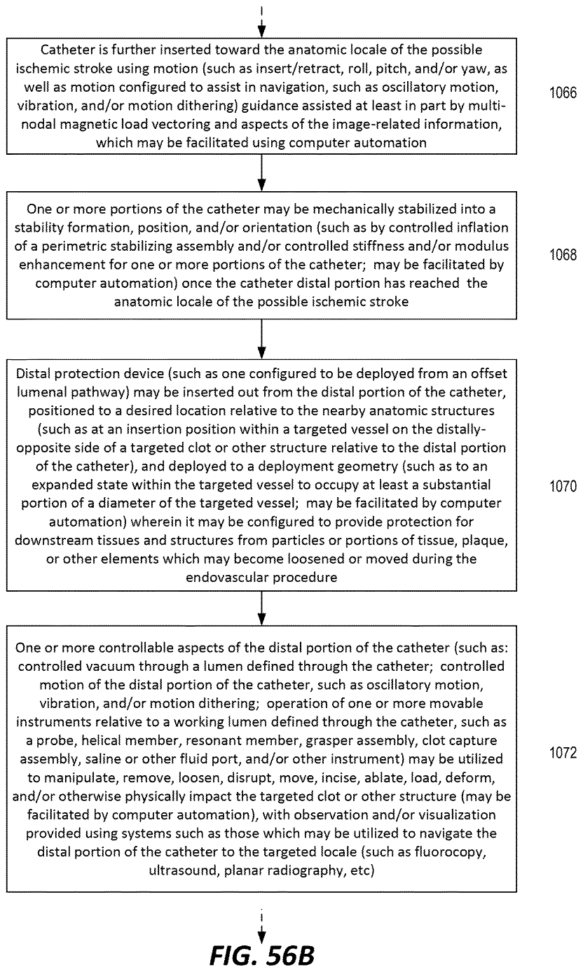

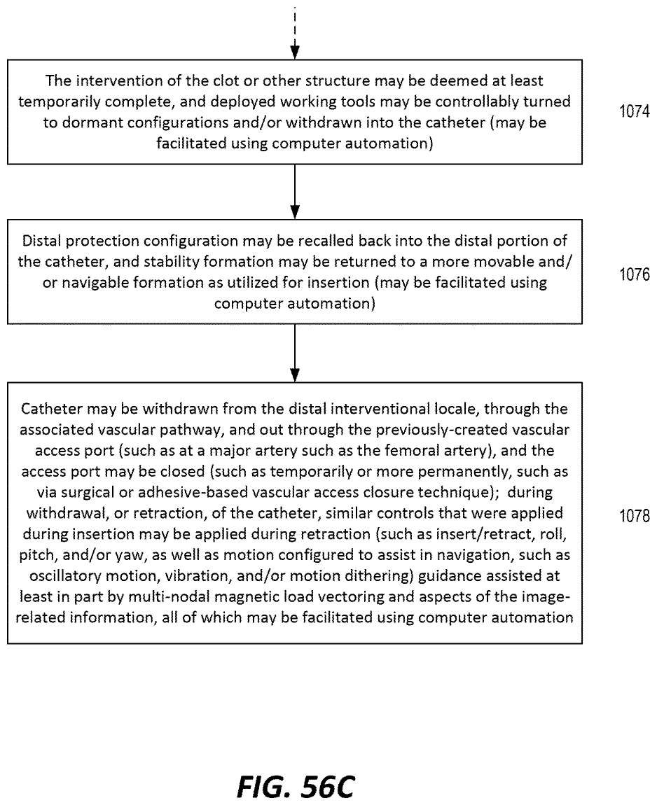

Referring to FIG. 56A, a patient may present with possible ischemic stroke conditions (1052). A relatively low-latency imaging study (such as MR, CT, planar radiography, and/or ultrasound) is conducted to assist in diagnostic evaluation of patient (1054). Image-related information may be analyzed and stored; may be further processed in background by associated computing resources (such as interpolation, stitching, and/or registration between image portions and/or modalities, which may be facilitated and/or enhanced via computer automation); system may be configured to assist operators in determining whether additional image-related information is required or would be helpful, such as by additional image data acquisition pertaining to a certain portion of the anatomy or one or more tissue structures (1056). Clinical opinion may be developed based at least in part upon image-related information; interventional catheterization may be deemed indicated or not (1058). With interventional catheterization deemed to be indicated, operators may prepare for electromechanical catheterization via multi-nodal magnetic navigation (such as by planning and/or prescribing a path for catheter and/or instrument introduction, selecting keep-out zones, keep-away structures, and/or loading/contact prioritization) (1060). A vascular access point may be selected (and such selection/planning may be assisted via computer automation based at least in part upon factors such as anatomic geometry (which may be based at least in part upon the image-related information gathered), instrument configuration (such as geometry, stiffness, bulk modulus, material properties such as external surface lubricity or roughness, etc), and prescribed instrument pathway, and/or keep out/away zones or structures and/or loading/contact prioritization), and initial surgical pathway may be established with introduction of a guiding member such as a guidewire (1062). With a guiding member in place, multi-nodal magnetically navigated catheter distal portion may be inserted, such as in an “over-the-wire” configuration utilizing a lumen defined through the catheter, and during insertion, magnetic guidance may be provided (such as via computer automation) to assist in minimizing interfacial loading between the catheter and the subject vascular-related tissue structures (such as the vascular walls, and/or plaques or other structures which may be adhered to or comprise the vascular walls) (1064).

Referring to FIG. 56B and continuing from FIG. 56A, the elongate instrument or catheter may be further inserted toward the anatomic locale of the possible ischemic stroke using motion (such as insert/retract, roll, pitch, and/or yaw, as well as motion configured to assist in navigation, such as oscillatory motion, vibration, and/or motion dithering) guidance assisted at least in part by multi-nodal magnetic load vectoring and aspects of the image-related information, which may be facilitated using computer automation (1066). One or more portions of the elongate instrument or catheter may be mechanically stabilized into a stability formation, position, and/or orientation (such as by controlled inflation of a perimetric stabilizing assembly and/or controlled stiffness and/or modulus enhancement for one or more portions of the catheter; may be facilitated by computer automation) once the catheter distal portion has reached the anatomic locale of the possible ischemic stroke (1068). A distal protection assembly or device (such as one configured to be deployed from an offset lumenal pathway) may be inserted out from the distal portion of the catheter, positioned to a desired location relative to the nearby anatomic structures (such as at an insertion position within a targeted vessel on the distally-opposite side of a targeted clot or other structure relative to the distal portion of the catheter), and deployed to a deployment geometry (such as to an expanded state within the targeted vessel to occupy at least a substantial portion of a diameter of the targeted vessel; may be facilitated by computer automation) wherein it may be configured to provide protection for downstream tissues and structures from particles or portions of tissue, plaque, or other elements which may become loosened or moved during the endovascular procedure (1070). One or more controllable aspects of the distal portion of the elongate instrument or catheter (such as: controlled vacuum through a lumen defined through the catheter; controlled motion of the distal portion of the catheter, such as oscillatory motion, vibration, and/or motion dithering; operation of one or more movable instruments relative to a working lumen defined through the catheter, such as a probe, helical member, resonant member, grasper assembly, clot capture assembly, saline or other fluid port, and/or other instrument) may be utilized to manipulate, remove, loosen, disrupt, move, incise, ablate, load, deform, and/or otherwise physically impact the targeted clot or other structure (may be facilitated by computer automation), with observation and/or visualization provided using systems such as those which may be utilized to navigate the distal portion of the catheter to the targeted locale (such as fluorocopy, ultrasound, planar radiography, etc) (1072).

Referring to FIG. 56C and continuing from FIG. 56B, the intervention of the clot or other structure may be deemed at least temporarily complete, and deployed working tools may be controllably turned to dormant configurations and/or withdrawn into the catheter (may be facilitated using computer automation) (1074). The distal protection assembly or device may be recalled back into the distal portion of the catheter, and stability formation may be returned to a more movable and/or navigable formation as utilized for insertion (may be facilitated using computer automation) (1076). The elongate instrument or catheter may be withdrawn from the distal interventional locale, through the associated vascular pathway, and out through the previously-created vascular access port (such as at a major artery such as the femoral artery), and the access port may be closed (such as temporarily or more permanently, such as via surgical or adhesive-based vascular access closure technique); during withdrawal, or retraction, of the catheter, similar controls that were applied during insertion may be applied during retraction (such as insert/retract, roll, pitch, and/or yaw, as well as motion configured to assist in navigation, such as oscillatory motion, vibration, and/or motion dithering) guidance assisted at least in part by multi-nodal magnetic load vectoring and aspects of the image-related information, all of which may be facilitated using computer automation (1078).

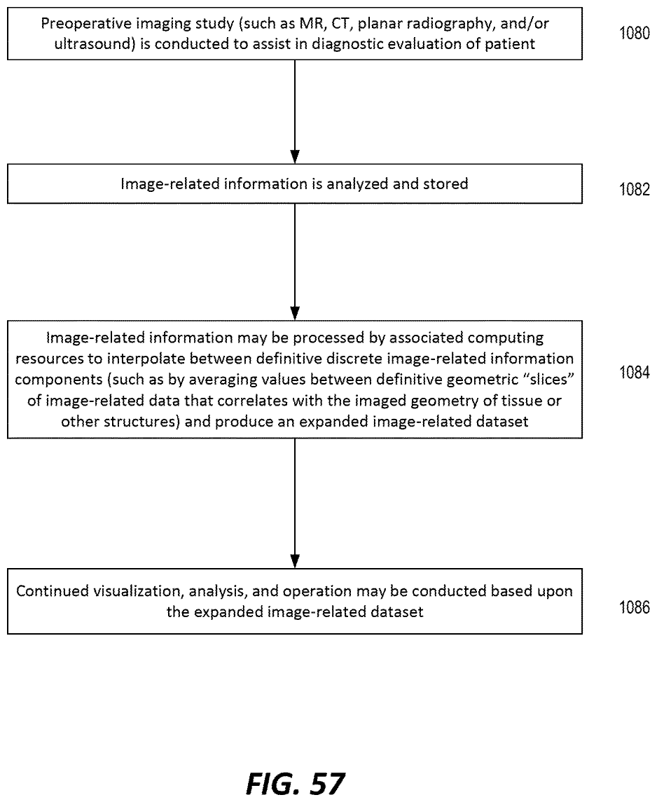

Referring to FIG. 57, a preoperative imaging study (such as MR, CT, planar radiography, and/or ultrasound) may be conducted to assist in diagnostic evaluation of patient (1080). Image-related information may be analyzed and/or stored (1082). Image-related information may be processed by associated computing resources to interpolate between definitive discrete image-related information components (such as by averaging values between definitive geometric “slices” of image-related data that correlates with the imaged geometry of tissue or other structures) and produce an expanded image-related dataset (1084). Continued visualization, analysis, and operation may be conducted based upon the expanded image-related dataset (1086).

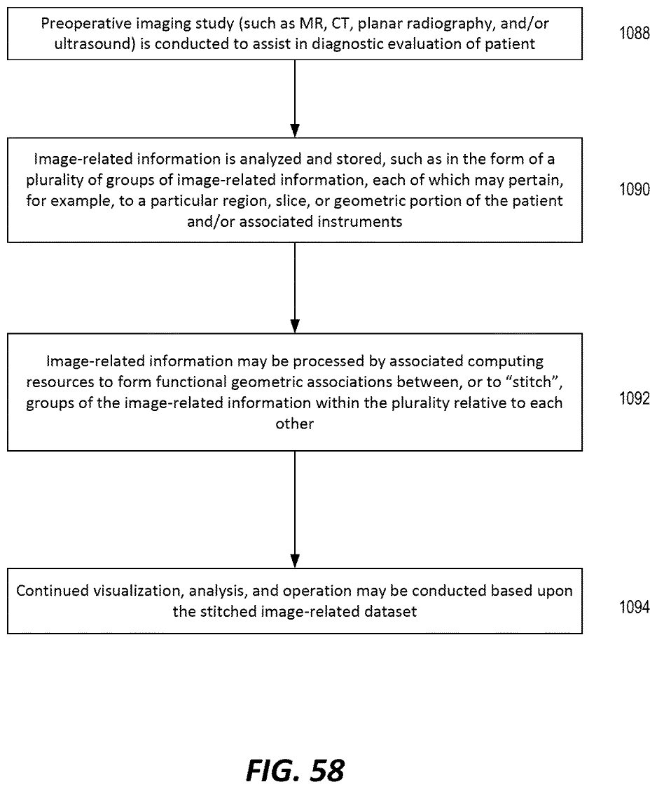

Referring to FIG. 58, a preoperative imaging study (such as MR, CT, planar radiography, and/or ultrasound) may be conducted to assist in diagnostic evaluation of patient (1088). Image-related information may be analyzed and/or stored, such as in the form of a plurality of groups of image-related information, each of which may pertain, for example, to a particular region, slice, or geometric portion of the patient and/or associated instruments (1090). Image-related information may be processed by associated computing resources to form functional geometric associations between, or to “stitch”, groups of the image-related information within the plurality relative to each other (1092). Continued visualization, analysis, and operation may be conducted based upon the stitched image-related dataset (1094).

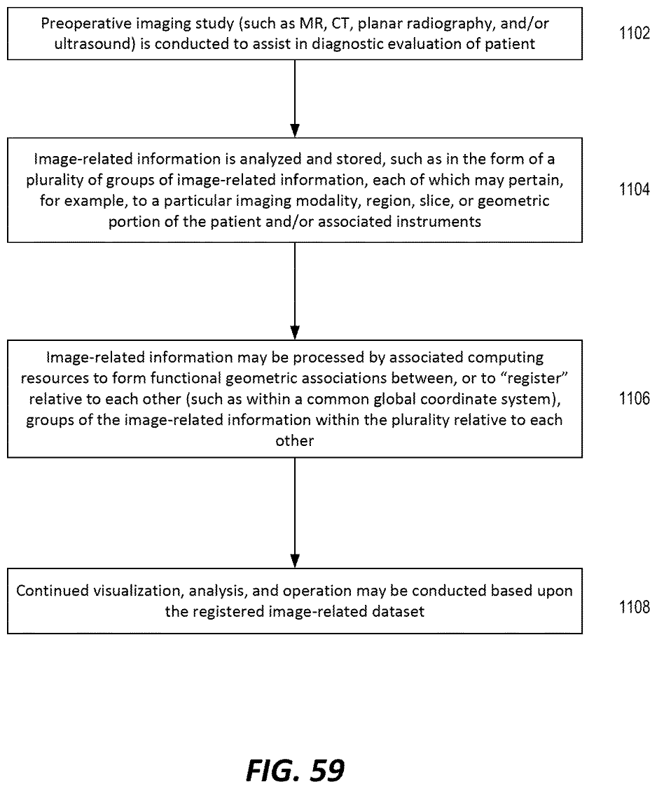

Referring to FIG. 59, a preoperative imaging study (such as MR, CT, planar radiography, and/or ultrasound) may be conducted to assist in diagnostic evaluation of patient (1104). Image-related information may be processed by associated computing resources to form functional geometric associations between, or to “register” relative to each other (such as within a common global coordinate system), groups of the image-related information within the plurality relative to each other (1106). Continued visualization, analysis, and operation may be conducted based upon the registered image-related dataset (1108).

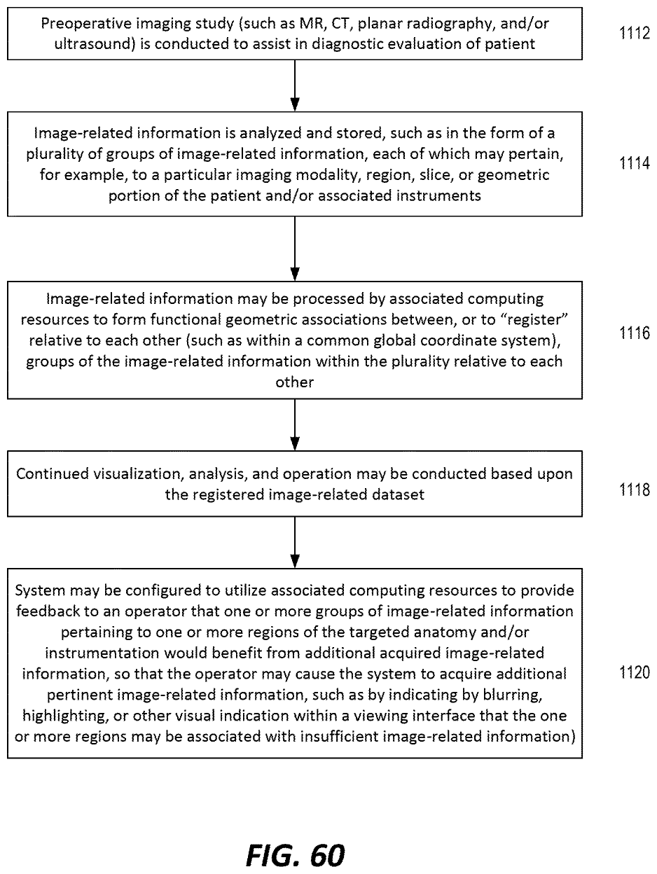

Referring to FIG. 60, a preoperative imaging study (such as MR, CT, planar radiography, and/or ultrasound) may be conducted to assist in diagnostic evaluation of patient (1112). Image-related information may be analyzed and stored, such as in the form of a plurality of groups of image-related information, each of which may pertain, for example, to a particular imaging modality, region, slice, or geometric portion of the patient and/or associated instruments (1114). Image-related information may be processed by associated computing resources to form functional geometric associations between, or to “register” relative to each other (such as within a common global coordinate system), groups of the image-related information within the plurality relative to each other (1116). Continued visualization, analysis, and operation may be conducted based upon the registered image-related dataset (1118). The system may be configured to utilize associated computing resources to provide feedback to an operator that one or more groups of image-related information pertaining to one or more regions of the targeted anatomy and/or instrumentation would benefit from additional acquired image-related information, so that the operator may cause the system to acquire additional pertinent image-related information, such as by indicating by blurring, highlighting, or other visual indication within a viewing interface that the one or more regions may be associated with insufficient image-related information) (1120).

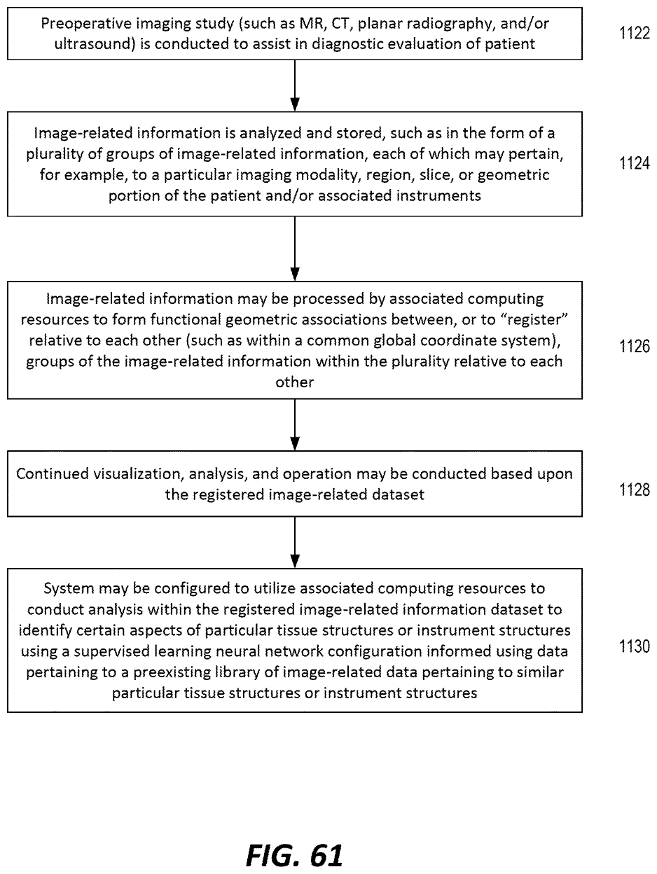

Referring to FIG. 61, a preoperative imaging study (such as MR, CT, planar radiography, and/or ultrasound) may be conducted to assist in diagnostic evaluation of patient (1122). Image-related information may be analyzed and stored, such as in the form of a plurality of groups of image-related information, each of which may pertain, for example, to a particular imaging modality, region, slice, or geometric portion of the patient and/or associated instruments (1124). Image-related information may be processed by associated computing resources to form functional geometric associations between, or to “register” relative to each other (such as within a common global coordinate system), groups of the image-related information within the plurality relative to each other (1126). Continued visualization, analysis, and operation may be conducted based upon the registered image-related dataset (1128). The system may be configured to utilize associated computing resources to conduct analysis within the registered image-related information dataset to identify certain aspects of particular tissue structures or instrument structures using a supervised learning neural network configuration informed using data pertaining to a preexisting library of image-related data pertaining to similar particular tissue structures or instrument structures (1130).

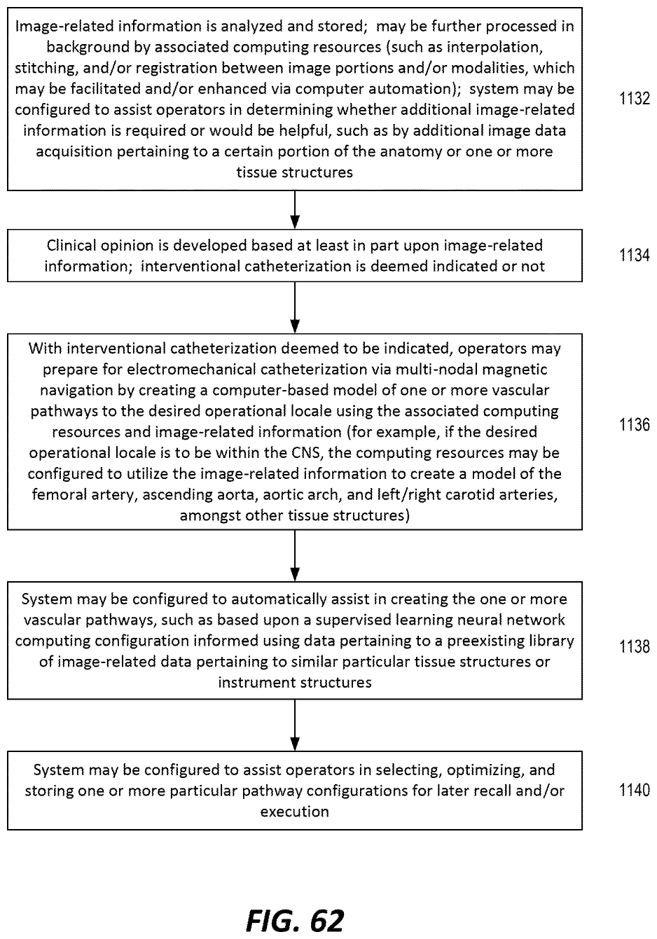

Referring to FIG. 62, image-related information may be analyzed and stored; further it may be processed in background by associated computing resources (such as interpolation, stitching, and/or registration between image portions and/or modalities, which may be facilitated and/or enhanced via computer automation); system may be configured to assist operators in determining whether additional image-related information is required or would be helpful, such as by additional image data acquisition pertaining to a certain portion of the anatomy or one or more tissue structures (1132). Clinical opinion may be developed based at least in part upon image-related information; interventional catheterization is deemed indicated or not (1134). With interventional catheterization deemed to be indicated, operators may prepare for electromechanical catheterization via multi-nodal magnetic navigation by creating a computer-based model of one or more vascular pathways to the desired operational locale using the associated computing resources and image-related information (for example, if the desired operational locale is to be within the CNS, the computing resources may be configured to utilize the image-related information to create a model of the femoral artery, ascending aorta, aortic arch, and left/right carotid arteries, amongst other tissue structures) (1136). The system may be configured to automatically assist in creating the one or more vascular pathways, such as based upon a supervised learning neural network computing configuration informed using data pertaining to a preexisting library of image-related data pertaining to similar particular tissue structures or instrument structures (1138). The system may be configured to assist operators in selecting, optimizing, and storing one or more particular pathway configurations for later recall and/or execution (1140).

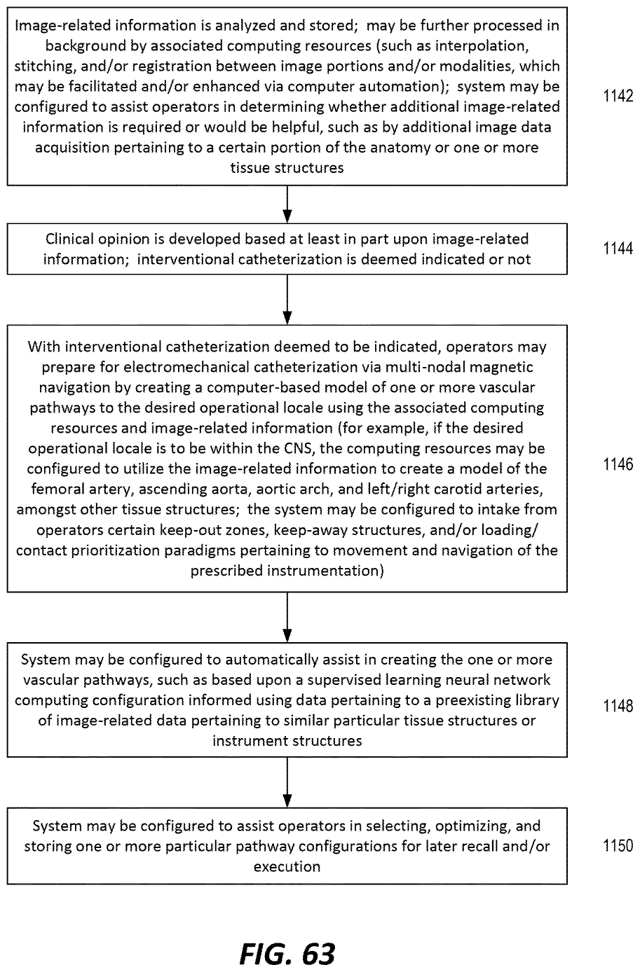

Referring to FIG. 63, image-related information may be analyzed and stored; further it may be processed in background by associated computing resources (such as interpolation, stitching, and/or registration between image portions and/or modalities, which may be facilitated and/or enhanced via computer automation); system may be configured to assist operators in determining whether additional image-related information is required or would be helpful, such as by additional image data acquisition pertaining to a certain portion of the anatomy or one or more tissue structures (1142). Clinical opinion may be developed based at least in part upon image-related information; interventional catheterization is deemed indicated or not (1144). With interventional catheterization deemed to be indicated, operators may prepare for electromechanical catheterization via multi-nodal magnetic navigation by creating a computer-based model of one or more vascular pathways to the desired operational locale using the associated computing resources and image-related information (for example, if the desired operational locale is to be within the CNS, the computing resources may be configured to utilize the image-related information to create a model of the femoral artery, ascending aorta, aortic arch, and left/right carotid arteries, amongst other tissue structures; the system may be configured to intake from operators certain keep-out zones, keep-away structures, and/or loading/contact prioritization paradigms pertaining to movement and navigation of the prescribed instrumentation) (1146). The system may be configured to automatically assist in creating the one or more vascular pathways, such as based upon a supervised learning neural network computing configuration informed using data pertaining to a preexisting library of image-related data pertaining to similar particular tissue structures or instrument structures (1148). The system may be configured to assist operators in selecting, optimizing, and storing one or more particular pathway configurations for later recall and/or execution (1150).

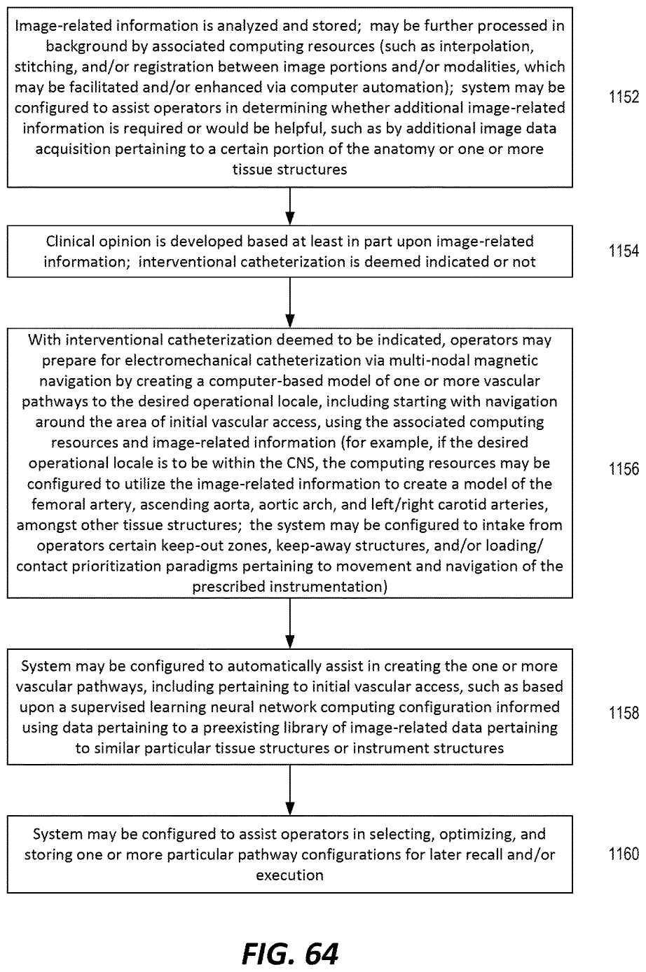

Referring to FIG. 64, image-related information may be analyzed and stored; further it may be processed in background by associated computing resources (such as interpolation, stitching, and/or registration between image portions and/or modalities, which may be facilitated and/or enhanced via computer automation); system may be configured to assist operators in determining whether additional image-related information is required or would be helpful, such as by additional image data acquisition pertaining to a certain portion of the anatomy or one or more tissue structures (1152). Clinical opinion may be developed based at least in part upon image-related information; interventional catheterization is deemed indicated or not (1154). With interventional catheterization deemed to be indicated, operators may prepare for electromechanical catheterization via multi-nodal magnetic navigation by creating a computer-based model of one or more vascular pathways to the desired operational locale, including starting with navigation around the area of initial vascular access, using the associated computing resources and image-related information (for example, if the desired operational locale is to be within the CNS, the computing resources may be configured to utilize the image-related information to create a model of the femoral artery, ascending aorta, aortic arch, and left/right carotid arteries, amongst other tissue structures; the system may be configured to intake from operators certain keep-out zones, keep-away structures, and/or loading/contact prioritization paradigms pertaining to movement and navigation of the prescribed instrumentation) (1156). The system may be configured to automatically assist in creating the one or more vascular pathways, including pertaining to initial vascular access, such as based upon a supervised learning neural network computing configuration informed using data pertaining to a preexisting library of image-related data pertaining to similar particular tissue structures or instrument structures (1158). The system may be configured to assist operators in selecting, optimizing, and storing one or more particular pathway configurations for later recall and/or execution (1160).

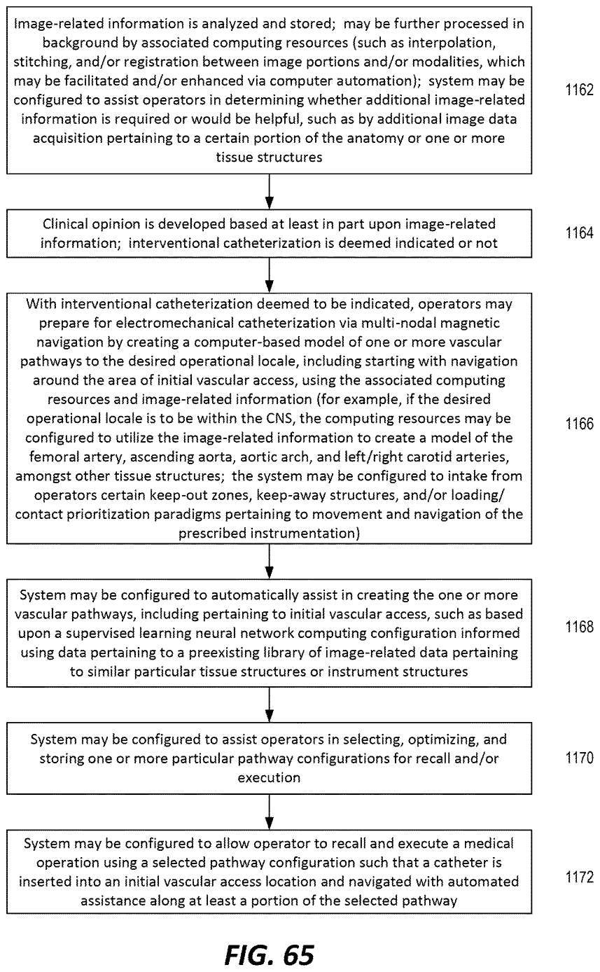

Referring to FIG. 65, image-related information may be analyzed and stored; further it may be processed in background by associated computing resources (such as interpolation, stitching, and/or registration between image portions and/or modalities, which may be facilitated and/or enhanced via computer automation); system may be configured to assist operators in determining whether additional image-related information is required or would be helpful, such as by additional image data acquisition pertaining to a certain portion of the anatomy or one or more tissue structures (1162). Clinical opinion may be developed based at least in part upon image-related information; interventional catheterization is deemed indicated or not (1164). With interventional catheterization deemed to be indicated, operators may prepare for electromechanical catheterization via multi-nodal magnetic navigation by creating a computer-based model of one or more vascular pathways to the desired operational locale, including starting with navigation around the area of initial vascular access, using the associated computing resources and image-related information (for example, if the desired operational locale is to be within the CNS, the computing resources may be configured to utilize the image-related information to create a model of the femoral artery, ascending aorta, aortic arch, and left/right carotid arteries, amongst other tissue structures; the system may be configured to intake from operators certain keep-out zones, keep-away structures, and/or loading/contact prioritization paradigms pertaining to movement and navigation of the prescribed instrumentation) (1166). The system may be configured to automatically assist in creating the one or more vascular pathways, including pertaining to initial vascular access, such as based upon a supervised learning neural network computing configuration informed using data pertaining to a preexisting library of image-related data pertaining to similar particular tissue structures or instrument structures (1168). The system may be configured to assist operators in selecting, optimizing, and storing one or more particular pathway configurations for recall and/or execution (1170). The system may be configured to allow an operator to recall and execute a medical operation using a selected pathway configuration such that a catheter is inserted into an initial vascular access location and navigated with automated assistance along at least a portion of the selected pathway (1172).

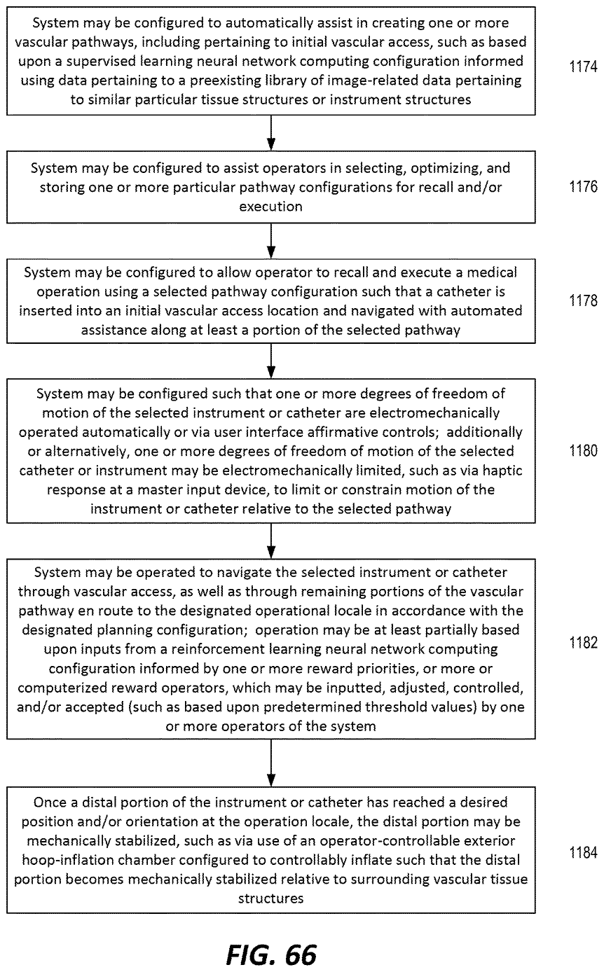

Referring to FIG. 66, a system may be configured to automatically assist in creating one or more vascular pathways, including pertaining to initial vascular access, such as based upon a supervised learning neural network computing configuration informed using data pertaining to a preexisting library of image-related data pertaining to similar particular tissue structures or instrument structures (1174). The system may be configured to assist operators in selecting, optimizing, and storing one or more particular pathway configurations for recall and/or execution (1176). The system may be configured to allow operator to recall and execute a medical operation using a selected pathway configuration such that a catheter is inserted into an initial vascular access location and navigated with automated assistance along at least a portion of the selected pathway (1178). The system may be configured such that one or more degrees of freedom of motion of the selected instrument or catheter are electromechanically operated automatically or via user interface affirmative controls; additionally or alternatively, one or more degrees of freedom of motion of the selected catheter or instrument may be electromechanically limited, such as via haptic response at a master input device, to limit or constrain motion of the instrument or catheter relative to the selected pathway (1180). The system may be operated to navigate the selected instrument or catheter through vascular access, as well as through remaining portions of the vascular pathway en route to the designated operational locale in accordance with the designated planning configuration; operation may be at least partially based upon inputs from a reinforcement learning neural network computing configuration informed by one or more reward priorities, or more or computerized reward operators, which may be inputted, adjusted, controlled, and/or accepted (such as based upon predetermined threshold values) by one or more operators of the system (1182). Once a distal portion of the instrument or catheter has reached a desired position and/or orientation at the operation locale, the distal portion may be mechanically stabilized, such as via use of an operator-controllable exterior hoop-inflation chamber configured to controllably inflate such that the distal portion becomes mechanically stabilized relative to surrounding vascular tissue Structures (1184).

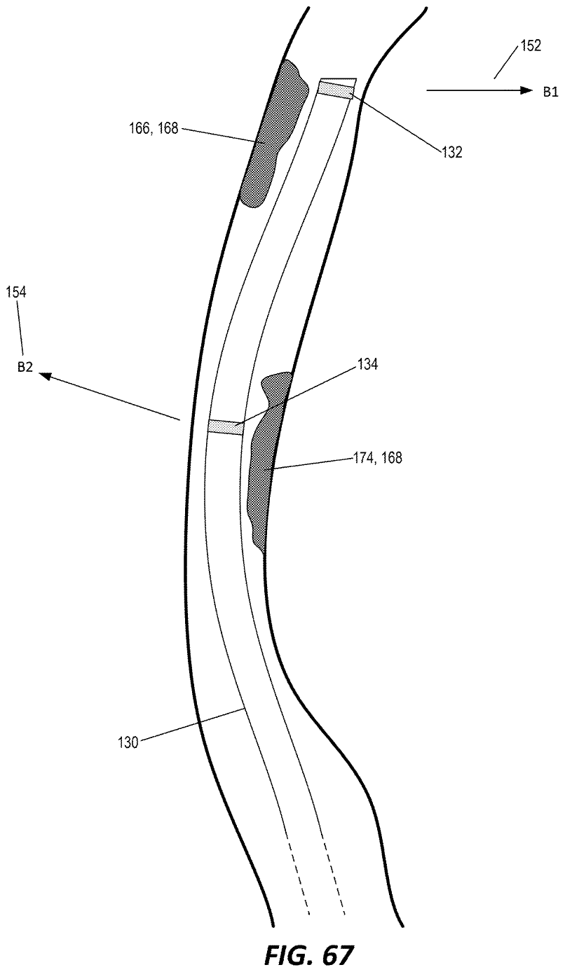

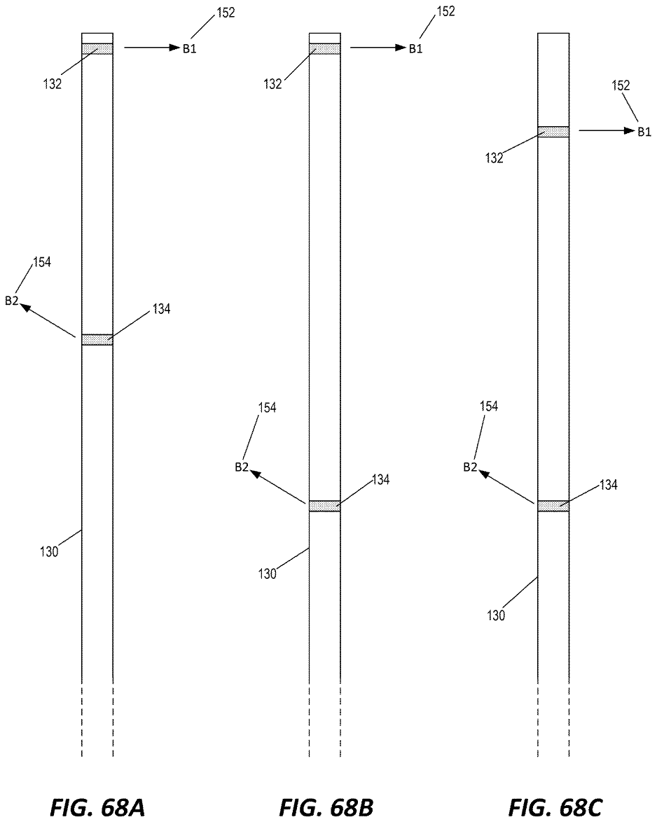

Referring to FIG. 67, as discussed above, in various embodiments it may be valuable to have multi-nodal (i.e., a plurality of steering nodes) steerability to navigate anatomy and avoid certain objects and/or obstacles. For example, as shown in FIG. 67, a first magnetic element steerability node (132) is shown distally positioned on the elongate instrument (130) and being guided, with the help of an applied and vectored magnetic field or magnetic flux (152), through the anatomy without colliding with an endoluminal object (166) such as a potentially unstable plaque (168); simultaneously, a second magnetic element steerability node (134) is shown more proximally positioned on the elongate instrument (130) and being guided, with the help of an applied and vectored magnetic field or magnetic flux (154), through the anatomy without colliding with a second endoluminal object (174) such as another potentially unstable plaque (168). Referring to FIGS. 68A-68C, it may be valuable in certain multi-nodal magnetic steerability configurations to dynamically change the longitudinal positioning of one or more of the steerability elements (132, 134). For example, the steering performance of the configuration in FIG. 68A would differ from that of FIG. 68B (with the proximal steerability element moved more proximally), and from that of FIG. 68C (similar to that of FIG. 68B, but with the distal steerability element 132 moved proximally).

Referring to FIGS. 69A and 69B, the physical integration of the magnetic steerability elements and the walls of the instrument (130) that surround them may be configured to provide for longitudinal adjustability. For example, as shown in FIG. 69A, the steerability elements (132, 134, for example) may be geometrically dimensioned such that when aligned rectilinearly (1204) as with the first steerability element depicted (132), they may be moved relative to the tubular structure of the elongate instrument (130), but when rotated (1210) out of rectilinear alignment (i.e., such as by an applied and vectored magnetic field intentionally configured to cause such rotation), they may be longitudinally locked or wedged in place (1208) relative to the inner surface (1202) of the elongate instrument (130). FIG. 69B illustrates a close-up view of just the keyed or textured innere interface surface which may be configured to have such lockability functionality (1202).

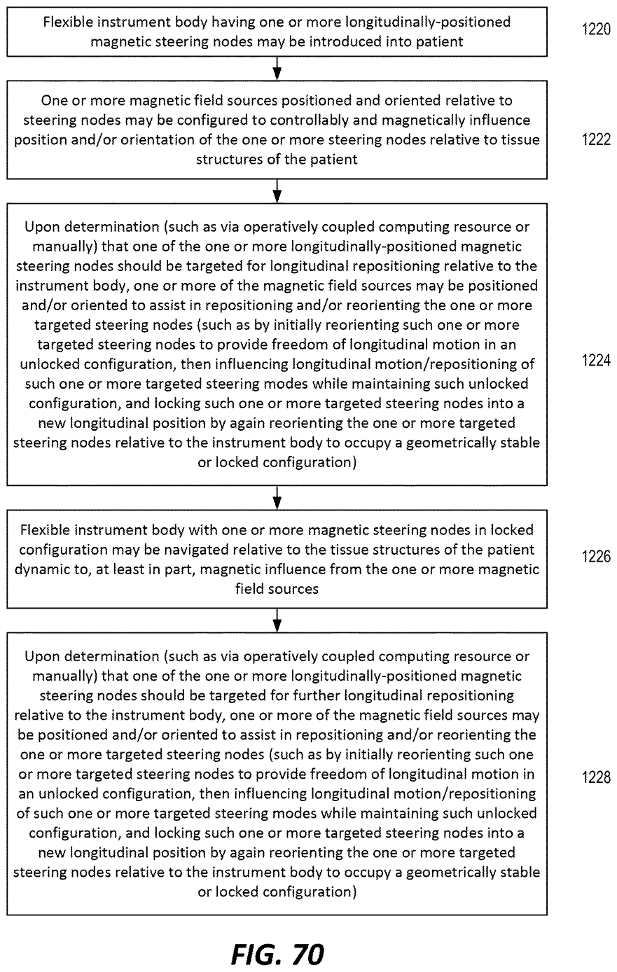

Thus referring to FIG. 70, a flexible instrument body having one or more longitudinally-positioned magnetic steering nodes may be introduced into patient (1220). One or more magnetic field sources positioned and oriented relative to steering nodes may be configured to controllably and magnetically influence position and/or orientation of the one or more steering nodes relative to tissue structures of the patient (1222). Upon determination (such as via operatively coupled computing resource or manually) that one of the one or more longitudinally-positioned magnetic steering nodes should be targeted for longitudinal repositioning relative to the instrument body, one or more of the magnetic field sources may be positioned and/or oriented to assist in repositioning and/or reorienting the one or more targeted steering nodes (such as by initially reorienting such one or more targeted steering nodes to provide freedom of longitudinal motion in an unlocked configuration, then influencing longitudinal motion/repositioning of such one or more targeted steering modes while maintaining such unlocked configuration, and locking such one more targeted steering nodes into a new longitudinal position by again reorienting the one or more targeted steering nodes relative to the instrument body to occupy a geometrically stable or locked configuration) (1224). The flexible instrument body with one or more magnetic steering nodes in locked configuration may be navigated relative to the tissue structures of the patient dynamic to, at least in part, magnetic influence from the one or more magnetic field sources (1226). Upon determination (such as via operatively coupled computing resource or manually) that one of the one or more longitudinally-positioned magnetic steering nodes should be targeted for further longitudinal repositioning relative to the instrument body, one or more of the magnetic field sources may be positioned and/or oriented to assist in repositioning and/or reorienting the one or more targeted steering nodes (such as by initially reorienting such one or more targeted steering nodes to provide freedom of longitudinal motion in an unlocked configuration, then influencing longitudinal motion/repositioning of such one or more targeted steering modes while maintaining such unlocked configuration, and locking such one or more targeted steering nodes into a new longitudinal position by again reorienting the one or more targeted steering nodes relative to the instrument body to occupy a geometrically stable or locked configuration) (1228).

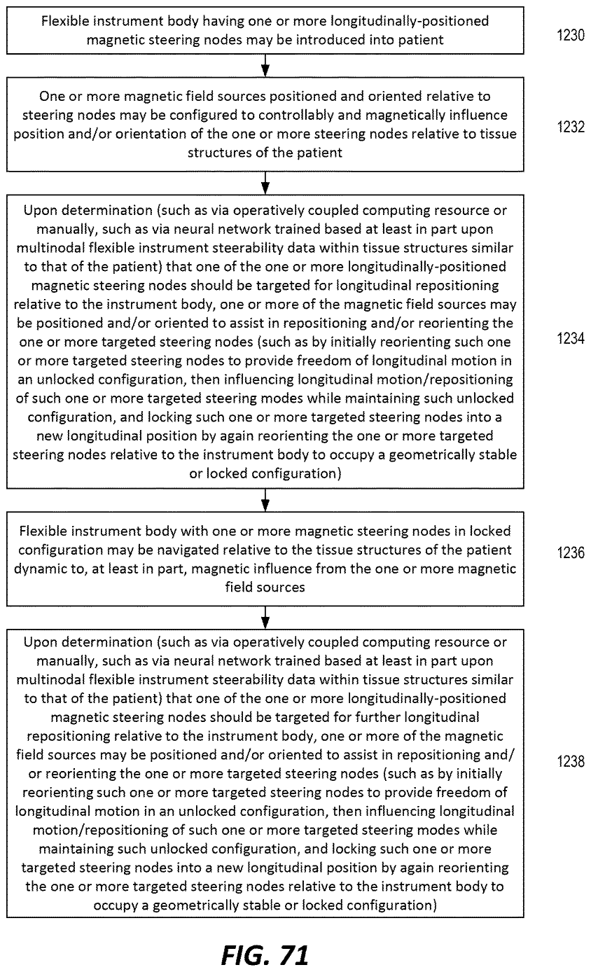

Referring to FIG. 71, a flexible instrument body having one or more longitudinally-positioned magnetic steering nodes may be introduced into patient (1230). One or more magnetic field sources positioned and oriented relative to steering nodes may be configured to controllably and magnetically influence position and/or orientation of the one or more steering nodes relative to tissue structures of the patient (1232). Upon determination (such as via operatively coupled computing resource or manually, such as via neural network trained based at least in part upon multinodal flexible instrument steerability data within tissue structures similar to that of the patient) that one of the one or more longitudinally-positioned magnetic steering nodes should be targeted for longitudinal repositioning relative to the instrument body, one or more of the magnetic field sources may be positioned and/or oriented to assist in repositioning and/or reorienting the one or more targeted steering nodes (such as by initially reorienting such one or more targeted steering nodes to provide freedom of longitudinal motion in an unlocked configuration, then influencing longitudinal motion/repositioning of such one or more targeted steering modes while maintaining such unlocked configuration, and locking such one or more targeted steering nodes into a new longitudinal position by again reorienting the one or more targeted steering nodes relative to the instrument body to occupy a geometrically stable or locked configuration) (1234). The flexible instrument body with one or more magnetic steering nodes in locked configuration may be navigated relative to the tissue structures of the patient dynamic to, at least in part, magnetic influence from the one or more magnetic field sources (1236). Upon determination (such as via operatively coupled computing resource or manually, such as via neural network trained based at least in part upon multinodal flexible instrument steerability data within tissue structures similar to that of the patient) that one of the one or more longitudinally-positioned magnetic steering nodes should be targeted for further longitudinal repositioning relative to the instrument body, one or more of the magnetic field sources may be positioned and/or oriented to assist in repositioning and/or reorienting the one or more targeted steering nodes (such as by initially reorienting such one or more targeted steering nodes to provide freedom of longitudinal motion in an unlocked configuration, then influencing longitudinal motion/repositioning of such one or more targeted steering modes while maintaining such unlocked configuration, and locking such one or more targeted steering nodes into a new longitudinal position by again reorienting the one or more targeted steering nodes relative to the instrument body to occupy a geometrically stable or locked configuration) (1238).

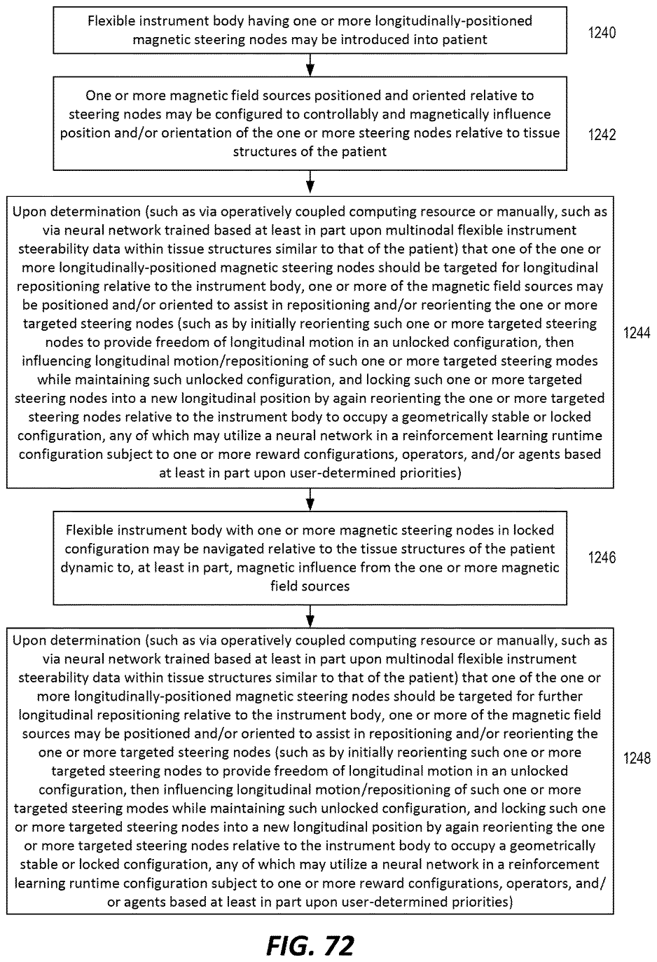

Referring to FIG. 72, a flexible instrument body having one or more longitudinally-positioned magnetic steering nodes may be introduced into patient (1240). One or more magnetic field sources positioned and oriented relative to steering nodes may be configured to controllably and magnetically influence position and/or orientation of the one or more steering nodes relative to tissue structures of the patient (1242). Upon determination (such as via operatively coupled computing resource or manually, such as via neural network trained based at least in part upon multinodal flexible instrument steerability data within tissue structures similar to that of the patient) that one of the one or more longitudinally-positioned magnetic steering nodes should be targeted for longitudinal repositioning relative to the instrument body, one or more of the magnetic field sources may be positioned and/or oriented to assist in repositioning and/or reorienting the one or more targeted steering nodes (such as by initially reorienting such one or more targeted steering nodes to provide freedom of longitudinal motion in an unlocked configuration, then influencing longitudinal motion/repositioning of such one or more targeted steering modes while maintaining such unlocked configuration, and locking such one or more targeted steering nodes into a new longitudinal position by again reorienting the one or more targeted steering nodes relative to the instrument body to occupy a geometrically stable or locked configuration, any of which may utilize a neural network in a reinforcement learning runtime configuration subject to one or more reward configurations, operators, and/or agents based at least in part upon user-determined priorities) (1244). A flexible instrument body with one or more magnetic steering nodes in locked configuration may be navigated relative to the tissue structures of the patient dynamic to, at least in part, magnetic influence from the one or more magnetic field sources (1246). Upon determination (such as via operatively coupled computing resource or manually, such as via neural network trained based at least in part upon multinodal flexible instrument steerability data within tissue structures similar to that of the patient) that one of the one or more longitudinally-positioned magnetic steering nodes should be targeted for further longitudinal repositioning relative to the instrument body, one or more of the magnetic field sources may be positioned and/or oriented to assist in repositioning and/or reorienting the one or more targeted steering nodes (such as by initially reorienting such one or more targeted steering nodes to provide freedom of longitudinal motion in an unlocked configuration, then influencing longitudinal motion/repositioning of such one or more targeted steering modes while maintaining such unlocked configuration, and locking such one or more targeted steering nodes into a new longitudinal position by again reorienting the one or more targeted steering nodes relative to the instrument body to occupy a geometrically stable or locked configuration, any of which may utilize a neural network in a reinforcement learning runtime configuration subject to one or more reward configurations, operators, and/or agents based at least in part upon user-determined priorities) (1248).

Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

The invention includes methods that may be performed using the subject devices. The methods may comprise the act of providing such a suitable device. Such provision may be performed by the end user. In other words, the “providing” act merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. As for other details of the present invention, these may be appreciated in connection with the above-referenced patents and publications as well as generally known or appreciated by those with skill in the art. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element—irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure.

1. An elongate instrument navigation system for conducting a medical procedure on a patient, comprising:
a. an elongate instrument comprising an elongate body coupled to two or more discretely positioned magnetic steering nodes, the elongate instrument comprising an insertion length selected such that a distal portion may be inserted to reach a position of a targeted interventional structure within the body of the patient, and a cross-sectional size profile configured to be navigated through one or more tissue structures of the patient as the patient is positioned in an operating environment which may be characterized by a global coordinate system;
b. a computing system operatively coupled to the elongate instrument and configured to present a control interface to an operator; and
c. two or more magnetic field sources, each of which is controllably movably coupled relative to the patient by a robotic coupling assembly operatively coupled to the computing system;
wherein in response to commands provided by the operator to the computing system, the computing system is configured to precisely navigate a portion of the elongate body of the elongate instrument relative to the one or more tissue structures of the patient by causing each of the two or more magnetic field sources to be positioned and oriented relative to the patient and relative to the two or more magnetic steering nodes of the elongate instrument using each of the robotic coupling assemblies.

2. The system of claim 1, wherein the computing system is configured to control magnetic field emissions from the two or more magnetic field sources.

3. The system of claim 2, wherein the computing system is configured to control magnetic field emissions from the two or more magnetic field sources independently.

4. The system of claim 1, wherein at least one robotic coupling assembly comprises a robotic arm.

5. The system of claim 4, wherein the robotic arm comprises one or more electromechanically controllable degrees of freedom.

6. The system of claim 1, further comprising an electromechanical insertion/retraction interface configured to controllably cause the elongate body to controllably insert or retract relative to the patient.

7. The system of claim 6, wherein the electromechanical insertion/retraction interface comprises one or more engagement wheels operably coupled between one or more drive motors and an outer aspect of the elongate body of the elongate instrument.

8. The system of claim 1, further comprising an electromechanical roll interface configured to controllably cause the elongate body to controllably roll relative to the patient.

9. The system of claim 8, wherein the electromechanical roll interface comprises one or more engagement wheels operably coupled between one or more drive motors and an outer aspect of the elongate body of the elongate instrument.

10. The system of claim 1, further comprising one or more ultrasound transducers operatively coupled to the computing system and configured to capture images pertaining to the location of one or more of the magnetic steering nodes relative to the one or more tissue structures of the patient.

11. The system of claim 10, wherein at least one of the one or more ultrasound transducers is coupled to at least one of the magnetic field sources.

12. The system of claim 1, further comprising one or more image capture devices operatively coupled to the computing system and configured to provide image information pertinent to the position or orientation of at least one of the magnetic field sources.

13. The system of claim 1, wherein the elongate instrument comprises one or more encoded portions configured such that they may be electronically observed by an operatively coupled encoder reader to determine a position or orientation of an encoded portion relative to the encoder reader.

14. The system of claim 13, wherein at least one of the one or more encoded portions is positioned longitudinally relative to the elongate instrument.

15. The system of claim 13, wherein at least one of the one or more encoded portions is positioned radially relative to the elongate instrument.

16. The system of claim 1, wherein the computing system is operatively coupled to a fluoroscopy system configured to provide image information pertaining to the elongate instrument and adjacent tissue structures of the patient.

17. The system of claim 1, wherein the computing system is operatively coupled to a radiography system configured to provide image information pertaining to the elongate instrument and adjacent tissue structures of the patient.

18. The system of claim 1, further comprising one or more electromagnetic localization sensors coupled to one or more locations along the elongate instrument, the sensors operatively coupled with a localization transmitter operatively coupled to the computing system and configured to determine localization information pertaining to the one or more electromagnetic localization sensors relative to transmissions from the localization transmitter.

19. The system of claim 18, wherein the one or more localization sensors comprises one or more receiving coils operatively coupled to the localization transmitter and computing system and configured for determining position and orientation of the one or more localization sensors in three dimensional space relative to the localization transmitter.

20. The system of claim 1, comprising two magnetic steering nodes and two magnetic field sources.

21. The system of claim 20, wherein each of the magnetic field sources is primarily directed to one of the two magnetic steering nodes.

22. The system of claim 21, wherein the computing system is configured to control shape and position of the elongate instrument in view of both magnetic field sources, each of which may influence the position of each of the magnetic steering nodes due to overlap patterns of each of the magnetic field sources.

23. The system of claim 21, wherein each of the two magnetic steering nodes is positioned discretely apart such that each of the magnetic field sources may be positioned and oriented by each of the robotic coupling assemblies to be only substantially influential upon one of the two magnetic steering nodes.

24. The system of claim 1, wherein at least one of the two or more magnetic steering nodes comprises a ferromagnetic material.

25. The system of claim 24, wherein the ferromagnetic material comprises iron, steel, and iron alloys.

26. The system of claim 1, wherein at least one of the two or more magnetic steering nodes comprises a paramagnetic material.

27. The system of claim 26, wherein the paramagnetic material comprises stainless steel, platinum, aluminum, magnesium, and lithium.

28. The system of claim 1, wherein at least one of the two or more magnetic steering nodes comprises a ring geometry with an aperture defined therethrough.

29. The system of claim 28, wherein at least one of the two or more magnetic steering nodes comprises a radially homogeneous construct.

30. The system of claim 28, wherein at least one of the two or more magnetic steering nodes comprises a circumferentially homogeneous construct.

31. The system of claim 28, wherein at least one of the two or more magnetic steering nodes comprises a radially non-homogeneous portion.

32. The system of claim 28, wherein at least one of the two or more magnetic steering nodes comprises a circumferentially non-homogeneous portion.

33. The system of claim 1, wherein at least one of the two or more magnetic field sources comprises a permanent magnet.

34. The system of claim 33, wherein the permanent magnet comprises a material selected from the list consisting of: iron, iron alloy, steel, nickel, cobalt, neodymium, and samarium.

35. The system of claim 33, wherein the permanent magnet is selected to have a field shape geometry.

36. The system of claim 35, wherein the field shape geometry is selected from the group consisting of: wide, narrow, and irregular.

37. The system of claim 35, wherein the field shape geometry is configured to be discrete relative to an outer shape of the permanent magnet.

38. The system of claim 1, wherein at least one of the two or more magnetic field sources comprises an electromagnet coil circuit.

39. The system of claim 38, wherein the electromagnet coil circuit comprises copper or gold.

40. The system of claim 38, wherein the electromagnet coil circuit comprises a substantially circular coil pattern.

41. The system of claim 1, wherein at least one of the two or more magnetic steering nodes comprises a controllably adjustable amount of magnetism.

42. The system of claim 1, wherein at least one of the two or more magnetic steering nodes may be controllably adjusted in longitudinal position relative to the elongate body of the elongate instrument.

43. The system of claim 1, wherein the elongate instrument comprises an engagement portion selected from the group consisting of: a resonant member assembly; a radiation emission assembly; an open interior cannulation volume; a controlled vacuum lumen; a fluid perfusion lumen; a flow redirection portion; a controllably actuated scissor tool; a controllably actuated grasper tool; a capture/agitation mesh interface; a retractable loose capture forked structure; and a helical thrombectomy assembly.

44. The system of claim 1, wherein the computing system comprises a neural network informed by preoperative information pertaining to the patient, operational selections made by the operator through the control interface preoperatively, and operational selections made by the operator intraoperatively through the control interface.

45. The system of claim 44, wherein the preoperative information pertaining to the patient is selected from the group consisting of: two-dimensional image data pertaining to the tissue structures of the patient; three-dimensional image data pertaining to the tissue structures of the patient; information pertaining to prior interventions; locations and geometries of potential hazards within the patient anatomy.

46. The system of claim 44, wherein the operational selections made by the operator through the control interface preoperatively comprise a provisional access pathway to a targeted tissue structure.

47. The system of claim 46, wherein the provisional access pathway is generated at least in part using automated calculations from the computing system based at least in part upon the preoperative information pertaining to the patient and operational selections made by the operator through the control interface preoperatively.

48. The system of claim 44, wherein the control interface to the operator may be operated at least in part via natural language commands from the operator interpreted by the computing system.

49. The system of claim 44, wherein the neural network comprises a reinforcement learning model informed by one or more agents configured to provide operational instructions to operate the elongate instrument relative to the patient via control of the operatively coupled two or more magnetic steering nodes, two or more magnetic field sources, and associated robotic coupling assemblies in view of the preoperative information pertaining to the patient, operational selections made by the operator through the control interface preoperatively, and operational selections made by the operator intraoperatively through the control interface.

50. The system of claim 49, wherein the neural network comprises a multi-agent model configured to parallel-process operation of the elongate instrument relative to the patient in view of a plurality of reward and priority configurations pertinent to a plurality of agents comprising the multi-agent model.

51. The system of claim 50, wherein the neural network also is configured to be multi-modal in operation, in that it is configured to have a plurality of modes of operating the elongate instrument relative to the patient in a time domain.

52. The system of claim 51, wherein the plurality of modes comprises an insertion navigation mode relative to tissue structures of the patient and a controlled mechanical stimulation mode relative to tissue structures of the patient.

53. The system of claim 52, wherein the controlled mechanical stimulation mode is configured to provide controlled oscillatory motion of a portion of the elongate instrument relative to a targeted tissue structure.

54. The system of claim 53, wherein the elongate instrument is deployed within the vasculature of the patient, and wherein controlled oscillatory motion of a portion of the elongate instrument relative to a targeted tissue structure comprises oscillatory motion selected to disrupt a thrombus or clot structure positioned within the vasculature for removal in a potential cardiovascular stroke paradigm.

55.-108. (canceled)
